(19) United States
(12) Patent Application Publication
(10) Pub. No.: US 2008/0234384 A1
(43) Pub. Date: Sep. 25, 2008

(54) BIARYL AND BIHETEROARYL COMPOUNDS USEFUL IN TREATING IRON DISORDERS

(75) Inventors: Mikhail Chafeev, Burnaby (CA); Nagasree Chakka, Burnaby (CA); Jean-Jacques Cadieux, Burnaby (CA); Jianmin Fu, Coquitlam (CA); Rajender Kamboj, Burnaby (CA); Vishnumurthy Kodumuru, Burnaby (CA); Jonathan Langille, Langley (CA); Shifeng Liu, Port Coquitlam (CA); Jianyu Sun, Richmond (CA); Serguei Sviridov, Burnaby (CA); Zaihui Zhang, Vancouver (CA)

Correspondence Address: Seed IP Law Group PLLC, 701 Fifth Avenue, Suite 5400, Seattle, WA, 98104, US

(73) Assignee: XENON PHARMACEUTICALS INC., Burnaby (CA)

(21) Appl. No.: 12/051,741

(22) Filed: Mar. 19, 2008

Related U.S. Application Data
(60) Provisional application No. 60/895,662, filed on Mar. 19, 2007.

(51) Int. Cl. A61K 31/166; C07C 335/32; A61K 31/155; C07C 257/00; C07C 279/04; C07C 233/78

(52) U.S. Cl. 514/616; 558/4; 514/636; 564/157; 564/236; 514/635; 564/243

(57) ABSTRACT

This invention is directed to compounds of formula (I):

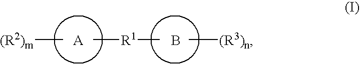

wherein

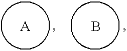

m, n, R¹, R² and R³ are as defined herein, as a stereoisomer, enantiomer, tautomer thereof or mixtures thereof; or a pharmaceutically acceptable salt, solvate or prodrug thereof, for the treatment of iron disorders. This invention is also directed to pharmaceutical compositions comprising the compounds and methods of using the compounds to treat iron disorders.

### CROSS-REFERENCE TO RELATED APPLICATION

This application claims the benefit under 37 U.S.C. § 119(e) of U.S. Provisional Patent Application No. 60/895,662, filed Mar. 19, 2007, which application is incorporated herein by reference in its entirety.

### FIELD OF THE INVENTION

The present invention is directed to biaryl and biheteroaryl compounds which are divalent metal transporter-1 inhibitors. The compounds of the invention, and pharmaceutical compositions comprising the compounds, are therefore useful in treating iron disorders in mammals.

### BACKGROUND OF THE INVENTION

Iron is an essential metal for life because it is a key constituent of a family of fundamental proteins, which includes hemoglobin, cytochromes, and NADH-coenzyme Q reductase. Maintaining body iron homeostasis is paramount to health because iron deficiency or excess results in morbidity and mortality.

Divalent metal transporter-1 (DMT1), also known as natural resistance-associated macrophage protein-2 (NRAMP2) and divalent cation transporter-1 (DCT1), is a ubiquitously expressed transmembrane protein involved in the maintenance of iron levels in the body. DMT1 is particularly important for iron absorption in the duodenum of the small intestine, where it is localized in the cytoplasm and brush border membrane of the villus enterocytes and mediates the influx of dietary non-heme iron from the intestinal lumen into the enterocytes (Gunshin et al., J. Clin. Invest., 2005, 115:1258-1266). Once dietary iron is absorbed across the intestinal wall, there is no physiologic mechanism for excreting iron from the body. Thus, excess absorbed iron is largely retained in the body and can accumulate throughout life. Excess accumulation of iron leads to considerable tissue damage and increased subsequent disease risk such as, for example, cirrhosis or hepatocellular carcinoma. Therefore, DMT1 is the primary focal point of controlling intestinal iron absorption for the maintenance of body iron homeostasis.

There is compelling evidence to support that DMT1 activity is tightly associated with many common diseases, such as, but not limited to, primary iron overload disorders, especially diseases related to hereditary hemochromatosis (Rolfs et al., Am. J. Physiol. Gastrointest. Liver Physiol., 2002, 282(4):G598-607). Further, DMT1 plays a significant role in intestinal iron hyperabsorption in patients suffering from hypochromic microcytic anemias and related disorders (Morgan et al., Blood Cell, Molecules, and Diseases, 2002, 29(3):384-399).

To date, there are only three known small-molecule, drug-like compounds that specifically modulate or inhibit DMT1 (Welti et al., Chem. Biol., 2006, 13:965-972). Accordingly, there is an unmet medical need to treat iron disorders, preferably primary iron overload and transfusional iron overload, including thalassemia, in mammals, preferably in humans, effectively and without adverse side effects. The present invention provides compounds and methods to meet these critical needs.

### SUMMARY OF THE INVENTION

The present invention is directed to biaryl and biheteroaryl compounds of the invention and pharmaceutical compositions comprising the compounds for the treatment of iron disorders.

Accordingly, in one aspect this invention provides compounds of formula (I):

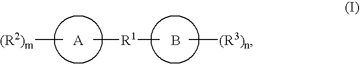

wherein:
n and m are each independently 1, 2, 3, 4, 5, 6 or 7;

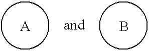

are each independently aryl or heteroaryl;
R¹ is a direct bond, —O—, —S(O)ₚ— (where p is 0, 1 or 2), —C(R⁴)₂—, —C(O)— or —N(R⁴)—;
at least one R² and at least one R³ is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
and the other R²'s and R³'s, if present, are each independently selected from the group consisting of alkyl, halo, haloalkyl, —R⁶—OR⁷, —R⁶—CN, —R⁶—NO₂—R⁶—N(R⁸)₂—R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, optionally substituted aryl, optionally substituted aralkyl, optionally substituted cycloalkyl, optionally substituted heterocyclyl, optionally substituted heteroaryl, or optionally substituted heteroaralkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl;
   as a stereoisomer, enantiomer, tautomer thereof or mixtures thereof;
   or a pharmaceutically acceptable salt, solvate or prodrug thereof.

In another aspect, the invention provides pharmaceutical compositions comprising a pharmaceutically acceptable excipient and a compound of formula (I), as a stereoisomer, enantiomer, tautomer thereof or mixtures thereof, or as a pharmaceutically acceptable salt, solvate or prodrug thereof.

In another aspect, the invention provides methods for treating an iron disorder in a mammal, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods for treating a disease or condition associated with an iron disorder in a mammal, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods for treating a disease or condition associated with an iron disorder in a mammal due to accumulation of iron in the body tissues of the mammal, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods for treating an iron disorder in a mammal or a disease or condition associated with an iron disorder in a mammal, wherein the iron disorder, disease or condition is associated with increased DMT1 activity and wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods of inhibiting the activity of DMT1 in a cell, preferably a mammalian cell, wherein the methods comprise contacting the mammalian cell with a DMT1-inhibitory amount of a compound of the invention, as set forth above, as a stereoisomer, enantiomer, tautomer thereof or mixtures thereof, or a pharmaceutically acceptable salt, solvate or prodrug thereof.

In another aspect, the invention provides methods of treating an iron disorder in a mammal, wherein the iron disorder is ameliorated by the inhibition of the activity of DMT1 in the mammal and wherein the methods comprise administering to the mammal a DMT1-inhibiting amount of a compound of the invention, as set forth above, as a stereoisomer, enantiomer, tautomer thereof or mixtures thereof, or a pharmaceutically acceptable salt, solvate or prodrug thereof, or a DMT1-inhibiting amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides pharmaceutical therapy in combination with one or more other compounds of the invention or one or more other accepted therapies or as any combination thereof to increase the potency of an existing or future drug therapy or to decrease the adverse events associated with the accepted therapy.

In one embodiment, the invention relates to a pharmaceutical composition combining compounds of the present invention with established or future therapies for the indications listed in the invention.

In another aspect, this invention is directed to the use of the compounds of the invention, as set forth above, as a stereoisomer, enantiomer, tautomer thereof or mixtures thereof, or a pharmaceutically acceptable salt, solvate or prodrug thereof, or the use of a pharmaceutical composition comprising a pharmaceutically acceptable excipient and a compound of the invention, as set forth above, as a stereoisomer, enantiomer, tautomer thereof or mixtures thereof, or a pharmaceutically acceptable salt, solvate or prodrug thereof, in the preparation of a medicament for the treatment of iron disorders in a mammal.

### DETAILED DESCRIPTION OF THE INVENTION

### Definitions

Certain chemical groups named herein may be preceded by a shorthand notation indicating the total number of carbon atoms that are to be found in the indicated chemical group. For example; C₇-C₁₂alkyl describes an alkyl group, as defined below, having a total of 7 to 12 carbon atoms, and C₄-C₁₂cycloalkylalkyl describes a cycloalkylalkyl group, as defined below, having a total of 4 to 12 carbon atoms. The total number of carbons in the shorthand notation does not include carbons that may exist in substituents of the group described.

In addition to the foregoing, as used in the specification and appended claims, unless specified to the contrary, the following terms have the meaning indicated:

“Amino” refers to the —NH₂ radical.

“Cyano” refers to the —CN radical.

“Hydroxy” refers to the —OH radical.

“Imino” refers to the ═NH substituent.

“Nitro” refers to the —NO₂ radical.

“Oxo” refers to the ═O substituent.

“Thioxo” refers to the ═S substituent.

“Trifluoromethyl” refers to the —CF₃ radical.

“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms, and which is attached to the rest of the molecule by a single bond, e.g., methyl, ethyl, n-propyl, 1-methylethyl(iso-propyl), n-butyl, n-pentyl, 1,1-dimethylethyl(t-butyl), 3-methylhexyl, 2-methylhexyl, and the like. Unless stated otherwise specifically in the specification, an alkyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkenyl” refers to a straight or branched hydrocarbon chain radical group consisting solely of carbon and hydrogen atoms, containing at least one double bond, having from two to twelve carbon atoms, preferably two to eight carbon atoms and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkynyl” refers to a straight or branched hydrocarbon chain radical group comprising solely of carbon and hydrogen atoms, containing at least one triple bond, optionally containing at least one double bond, having from two to twelve carbon atoms, preferably two to eight carbon atoms and which is attached to the rest of the molecule by a single bond, for example, ethynyl, propynyl, butynyl, pentynyl, hexynyl, and the like. Unless stated otherwise specifically in the specification, an alkynyl group may be optionally substituted by one or more of the following substituents: alkyl, alkenyl, halo, haloalkenyl, cyano, nitro, aryl, cycloalkyl, heterocyclyl, heteroaryl, oxo, trimethylsilanyl, —OR⁴, —OC(O)—R⁴, —N(R⁴)₂, —C(O)R⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkynylene” or “alkynylene chain” refers to a straight or branched divalent hydrocarbon chain linking the rest of the molecule to a radical group, consisting solely of carbon and hydrogen, containing at least one triple bond and having from two to twelve carbon atoms, e.g., propynylene, n-butynylene, and the like. The alkynylene chain is attached to the rest of the molecule through a single bond and to the radical group through a double bond or a single bond. The points of attachment of the alkynylene chain to the rest of the molecule and to the radical group can be through one carbon or any two carbons within the chain. Unless stated otherwise specifically in the specification, an alkynylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkoxy” refers to a radical of the formula —ORₐ where Rₐ is an alkyl radical as defined above containing one to twelve carbon atoms. The alkyl part of the alkoxy radical may be optionally substituted as defined above for an alkyl radical.

“Alkoxyalkyl” refers to a radical of the formula —R_{b}—O—Rₐ where R_{b} is an alkylene chain as defined above and Rₐ is an alkyl radical as defined above. The oxygen atom may be bonded to any carbon in the alkylene chain and in the alkyl radical. The alkyl part of the alkoxyalkyl radical may be optionally substituted as defined above for an alkyl group. The alkylene chain part of the alkoxyalkyl radical may be optionally substituted as defined above for an alkylene chain.

“Aryl” refers to a hydrocarbon ring system radical comprising hydrogen, 6 to 18 carbon atoms and at least one aromatic ring. For purposes of this invention, the aryl radical may be a monocyclic, bicyclic, tricyclic or tetracyclic ring system, which may included fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals optionally substituted by one or more substituents independently selected from the group consisting of alkyl, akenyl, halo, haloalkyl, haloalkenyl, cyano, nitro, aryl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁴, —R¹⁵—S(O)ₜOR¹⁶ (where t is 1 to 2), —R¹⁵—S(O)ₚR¹⁶ (where p is 0 to 2), and —R¹⁵—S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Aralkyl” refers to a radical of the formula —R_{b}—R_{c} where R_{b} is an alkylene chain as defined above and R_{c} is one or more aryl radicals as defined above, for example, benzyl, diphenylmethyl and the like. The alkylene chain part of the aralkyl radical may be optionally substituted as described above for an alkylene chain. The aryl part of the aralkyl radical may be optionally substituted as described above for an aryl group.

“Aralkenyl” refers to a radical of the formula —R_{d}—R_{c} where R_{d} is an alkenylene chain as defined above and R_{c} is one or more aryl radicals as defined above. The aryl part of the aralkenyl radical may be optionally substituted as described above for an aryl group. The alkenylene chain part of the aralkenyl radical may be optionally substituted as defined above for an alkenylene group.

“Aralkynyl” refers to a radical of the formula —RₑR_{c} where Rₑ is an alkynylene chain as defined above and R^{c} is one or more aryl radicals as defined above. The aryl part of the aralkynyl radical may be optionally substituted as described above for an aryl group. The alkynylene chain part of the aralkynyl radical may be optionally substituted as defined above for an alkynylene chain.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptly, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, the term “cycloalkyl” is meant to include cycloalkyl radicals which are optionally substituted by one or more substituents independently selected from the group consisting of alkyl, alkenyl, halo, haloalkyl, haloalkenyl, cyano, nitro, oxo, aryl, aralkyl, cycloalkyl, cycloalkylalkyl, heterocyclyl, heterocyclylalkyl, heteroaryl, heteroarylalkyl, —R¹⁵—R¹⁴, —R¹⁵—OC(O)—R¹⁴, —R¹⁵N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁴, —R¹⁵—S(O)ₜOR¹⁶ (where t is 1 to 2), —R¹⁵—S(O)ₚR¹⁶ (where p is 0 to 2), and —R¹⁵—S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Cycloalkylalkyl” refers to a radical of the formula —R_{b}R_{g} where R_{b} is an alkylene chain as defined above and R_{g} is a cycloalkyl radical as defined above. The alkylene chain and the cycloalkyl radical may be optionally substituted as defined above.

“Cycloalkylalkenyl” refers to a radical of the formula —R_{d}R_{g} where R_{d} is an alkenylene chain as defined above and R_{g} is a cycloalkyl radical as defined above. The alkenylene chain and the cycloalkyl radical may be optionally substituted as defined above.

“Cycloalkylalkynyl” refers to a radical of the formula —RₑR_{g} where Rₑ is an alkynylene radical as defined above and R_{g} is a cycloalkyl radical as defined above. The alkynylene chain and the cycloalkyl radical may be optionally substituted as defined above.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may be optionally substituted as defined above for an alkyl group.

“Haloalkenyl” refers to an alkenyl radical, as defined above, that is substituted by one or more halo radicals, as defined above. The alkenyl part of the haloalkyl radical may be optionally substituted as defined above for an alkenyl group.

“Haloalkynyl” refers to an alkynyl radical, as defined above, that is substituted by one or more halo radicals, as defined above. The alkynyl part of the haloalkyl radical may be optionally substituted as defined above for an alkynyl group.

“Heterocyclyl” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above which are optionally substituted by one or more substituents selected from the group consisting of alkyl, alkenyl, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁴, —R¹⁵—S(O)ₜOR¹⁶ (where t is 1 to 2), —R¹⁵—S(O)ₚR¹⁶ (where p is 0 to 2), and —R¹⁵—S(O)ₜN(R¹⁴)₂ (where t is 1 to 2) where each R¹⁴ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, alkenyl, haloalkyl, cycloalkyl, cycloalkylalkyl, aryl, aralkyl, heterocyclyl, heterocyclylalkyl, heteroaryl or heteroarylalkyl.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical may be optionally substituted as described above for heterocyclyl radicals.

“Heterocyclylalkyl” refers to a radical of the formula —R_{b}Rₕ where R_{b} is an alkylene chain as defined above and Rₕ is a heterocyclyl radical as defined above, and if the heterocyclyl is a nitrogen-containing heterocyclyl, the heterocyclyl may be attached to the alkyl radical at the nitrogen atom. The alkylene chain of the heterocyclylalkyl radical may be optionally substituted as defined above for an alkylene chain. The heterocyclyl part of the heterocyclylalkyl radical may be optionally substituted as defined above for a heterocyclyl group.

“Heterocyclylalkenyl” refers to a radical of the formula —R_{d}Rₕ where R_{d} is an alkenylene chain as defined above and Rₕ is a heterocyclyl radical as defined above, and if the heterocyclyl is a nitrogen-containing heterocyclyl, the heterocyclyl may be attached to the alkenylene chain at the nitrogen atom. The alkenylene chain of the heterocyclylalkenyl radical may be optionally substituted as defined above for an alkenylene chain. The heterocyclyl part of the heterocyclylalkenyl radical may be optionally substituted as defined above for a heterocyclyl group.

“Heterocyclylalkynyl” refers to a radical of the formula —RₑRₕ where Rₑ is an alkynylene chain as defined above and Rₕ is a heterocyclyl radical as defined above, and if the heterocyclyl is a nitrogen-containing heterocyclyl, the heterocyclyl may be attached to the alkynyl radical at the nitrogen atom. The alkynylene chain part of the heterocyclylalkynyl radical may be optionally substituted as defined above for an alkynylene chain. The heterocyclyl part of the heterocyclylalkynyl radical may be optionally substituted as defined above for a heterocyclyl group.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl(benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrrolyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, the term “heteroaryl” is meant to include heteroaryl radicals as defined above which are optionally substituted by one or more substituents selected from the group consisting of alkyl, alkenyl, alkoxy, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁴, —R¹⁵—S(O)ₜOR¹⁶ (where t is 1 to 2), —R¹⁵—S(O)ₚR¹⁶ (where p is 0 to 2), and —R¹⁵—S(O)ₜN(R¹⁴)₂ (where t is 1 to 2) where each R¹⁴ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, alkenyl, haloalkyl, cycloalkyl, cycloalkylalkyl, aryl, aralkyl, heterocyclyl, heterocyclylalkyl, heteroaryl or heteroarylalkyl.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical may be optionally substituted as described above for heteroaryl radicals.

“Heteroarylalkyl” refers to a radical of the formula —R_{b}Rᵢ where R_{b} is an alkylene chain as defined above and Rᵢ is a heteroaryl radical as defined above. The heteroaryl part of the heteroarylalkyl radical may be optionally substituted as defined above for a heteroaryl group. The alkylene chain part of the heteroarylalkyl radical may be optionally substituted as defined above for an alkylene chain.

“Heteroarylalkenyl” refers to a radical of the formula —R_{d}Rᵢ where R_{d} is an alkenylene chain as defined above and Rᵢ is a heteroaryl radical as defined above. The heteroaryl part of the heteroarylalkenyl radical may be optionally substituted as defined above for a heteroaryl group. The alkenylene chain part of the heteroarylalkenyl radical may be optionally substituted as defined above for an alkenylene chain.

“Heteroarylalkynyl” refers to a radical of the formula —RₑRᵢ where Rₑ is an alkynylene chain as defined above and Rᵢ is a heteroaryl radical as defined above. The heteroaryl part of the heteroarylalkynyl radical may be optionally substituted as defined above for a heteroaryl group. The alkynylene chain part of the heteroarylalkynyl radical may be optionally substituted as defined above for an alkynylene chain.

“Hydroxyalkyl” refers to an alkyl radical, as defined above, substituted by one or more hydroxy groups.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

The term “prodrug” is also meant to include any covalently bonded carriers, which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the invention and the like.

The invention disclosed herein is also meant to encompass all pharmaceutically acceptable compounds of formula (I) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the binding affinity to pharmacologically important site of action on DMT1. Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Preparations and Examples as set out below using an appropriate isotopically-labeled reagent in place of the non-labeled reagent previously employed.

The invention disclosed herein is also meant to encompass the in vivo metabolic products of the disclosed compounds. Such products may result from, for example, the oxidation, reducation, hydrolysis, amidation, esterification, and the like of the administered compound, primarily due to enzymatic processes. Accordingly, the invention includes compounds produced by a process comprising administering a compound of this invention to a mammal for a period of time sufficient to yield a metabolic product thereof. Such products are typically identified by administering a radiolabelled compound of the invention in a detectable dose to an animal, such as rat, mouse, guinea pig, monkey, or to human, allowing sufficient time for metabolism to occur, and isolating its coversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets, (e.g. cats, dogs, swine, cattle, sheep, goats, horses, rabbits), and non-domestic animals such as wildlife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution. When a functional group is described as “optionally substituted,” and in turn, substitutents on the functional group are also “optionally substituted” and so on, for the purposes of this invention, such iterations are limited to five, preferably such iterations are limited to two.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Therapeutically effective amount” refers to that amount of a compound of the invention which, when administered to a mammal, preferably a human, is sufficient to effect treatment, as defined below, of an iron disorder or a disease or condition associated with an iron disorder, in the mammal, preferably a human. The amount of a compound of the invention which constitutes a “therapeutically effective amount” will vary depending on the compound, the iron disorder, disease or condition and its severity, the manner of administration, and the age of the mammal to be treated, but can be determined routinely by one of ordinary skill in the art having regard to his own knowledge and to this disclosure. Preferably, for purposes of this invention, a “therapeutically effective amount” is that amount of a compound of invention which is sufficient to inhibit the activity of DMT1.

“Treating” or “treatment”, as used herein, covers the treatment of an iron disorder in a mammal, preferably a human, or a disease or condition associated with an iron disorder in a mammal, preferably a human, and includes:

(i) preventing an iron disorder in a mammal, or a disease or condition associated with an iron disorder in the mammal, from occurring in the mammal;

(ii) inhibiting an iron disorder in a mammal, or a disease or condition associated with an iron disorder in the mammal, i.e., arresting its development;

(iii) relieving an iron disorder in a mammal, or a disease or condition associated with an iron disorder in the mammal, i.e., causing regression of the iron disorder or the disease or condition;

(iv) relieving the symptoms of an iron disorder in a mammal, or a disease or condition associated with an iron disorder in the mammal, i.e., relieving the symptoms without addressing the underlying iron disorder, disease or condition; or

(v) restoring and/or maintaining normal serum iron levels, transferrin saturation, serum ferritin, liver iron and/or bodily iron levels in a mammal having an iron disorder or having a disease or condition associated with an iron disorder.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts may contain one or more asymmetric centres and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallisation. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

Also within the scope of the invention are intermediate compounds of formula (I) and all polymorphs of the aforementioned species and crystal habits thereof.

The chemical naming protocol and structure diagrams used herein are a modified form of the I.U.P.A.C. nomenclature system, using the ChemDraw Version 10 software naming program (CambridgeSoft), wherein the compounds of the invention are named herein as derivatives of the central core structure, e.g., the biaryl or biheteroaryl structure. For complex chemical names employed herein, a substituent group is named before the group to which it attaches. For example, cyclopropylethyl comprises an ethyl backbone with cyclopropyl substituent. In chemical structure diagrams, all bonds are identified, except for some carbon atoms, which are assumed to be bonded to sufficient hydrogen atoms to complete the valency.

The use of parentheses in substituent groups is used herein to conserve space. Accordingly, the use of parenthesis in a substituent group indicates that the group enclosed within the parentheses is attached directly to the atom preceding the parenthesis. For example, one of the choices for R¹ is the —R⁶—S—C(═NR⁴)N(R⁴)N(R⁴)R⁵ group. The formula for this group can be drawn as follows:

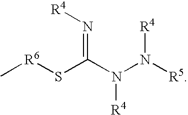

Thus, for example, a compound of formula (Ia), which is a compound of formula (I) as described herein, wherein R¹ is —S—; R^{2a} and R^{3a} are each (amino(imino)methyl)thiomethyl; R^{2b}, R^{2d}, R^{2e}, R^{3b}, R^{3d} and R^{3e} are each hydrogen; R^{2c} is fluoro and R^{3c} is trifluoromethyl; i.e., a compound of the following formula:

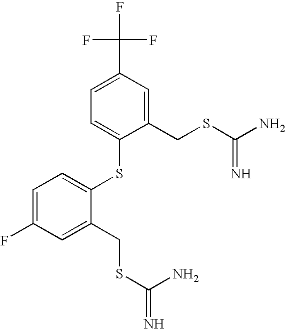

is named herein as 2-[2-(2-carbamimidoylsulfanylmethyl-4-fluorophenylsulfanyl)-5-(trifluoromethyl)benzyl]isothiourea.

### EMBODIMENTS OF THE INVENTION

Of the various aspects of the invention set forth above in the Summary of the Invention, certain embodiments are preferred.

Of the compounds of formula (I) as described above in the Summary of the Invention, one embodiment is the compounds of formula (Ia):

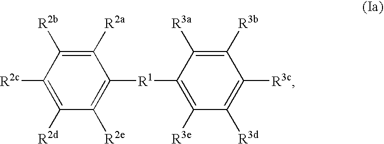

wherein:
R¹ is a direct bond, —O—, —S(O)ₚ— (where p is 0, 1 or 2), —C(R⁴)₂—, —C(O)— or —N(R⁴)—;
R^{2a}, R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2 and wherein at least one of R^{2a}, R^{2b}, R^{2c}, R^{2d} and R^{2e} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{3a}, R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵ wherein each t is independently 1 or 2 and each p is 0, 1 or 2 and wherein at least one of R^{3a}, R^{3b}, R^{3c}, R^{3d} and R^{3e} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
each R⁴ and R⁵ is independently hydrogen, alkyl, optionally substituted aryl, optionally substituted aralkyl, optionally substituted cycloalkyl, optionally substituted heterocyclyl, optionally substituted heteroaryl, or optionally substituted heteroaralkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), one embodiment is the compounds of formula (Ia) wherein:
R¹ is a direct bond;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is the compounds of formula (Ia) wherein:
R¹ is a direct bond;
R^{2a} and R^{3a} are the same and selected from the group consisting of —R⁶—S—C(═NR⁴)N(R⁴)R⁵, —R⁶—O—C(═NR⁴)N(R⁴)R⁵, —R⁶—C(═NR⁴)N(R⁴)R⁵ and —R⁶—N(R⁷)C(═NR⁴)N(R⁴)R⁵;
R^{2e} and R^{3e} are the same and selected from the group consisting of hydrogen, alkyl, —R⁶—C(O)OR⁸, —R⁶—C(O)N(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵ and —R⁶—N(R⁷)C(═NR⁴)N(R⁴)R⁵;
R^{2b}, R^{2c} and R^{2d} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R³ is independently hydrogen or alkyl; and
each R⁹ is alkyl.

Of the compounds of formula (Ia), another embodiment is the compounds of formula (Ia) wherein:
R¹ is a direct bond;
R^{2a} and R^{3a} are both —R⁶—S—C(═NR⁴)N(R⁴)R⁵;
R^{2e} and R^{3e} are the same and selected from the group consisting of hydrogen, alkyl, —R⁶—C(O)OR⁸ and —R⁶—S—C(═NR⁴)N(R⁴)R⁵;
R^{2b}, R^{2c} and R^{2d} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is the compounds of formula (Ia) wherein:
R¹ is a direct bond;
R^{2a} and R^{3a} are both —R⁶—S—C(═NR⁴)N(R⁴)R⁵;
R^{2e} and R^{3e} are the same and selected from the group consisting of hydrogen, alkyl, —R⁶—C(O)OR⁸ and —R⁶—S—C(═NR⁴)N(R⁴)R⁵;
R^{2b}, R^{2c} and R^{2d} are each independently selected from the group consisting of hydrogen, alkyl, halo and haloalkyl;
R^{3b}, R^{3c} and R^{3d} are each independently selected from the group consisting of hydrogen, alkyl, halo and haloalkyl;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R⁸ is hydrogen or alkyl.

One embodiment of the compounds of formula (Ia) is a compound selected from the group consisting of:
2-(2′-carbamimidoylsulfanylmethyl-biphenyl-2-ylmethyl)-isothiourea;
(6,6′-dimethylbiphenyl-2,2′-diyl)bis(methylene)dicarbamimidothioate dihydrobromide;
biphenyl-2,2′,6,6′-tetrayltetrakis(methylene)tetracarbamimidothioate; and
dimethyl 6,6′-bis(carbamimidoylthiomethyl)biphenyl-2,2′-dicarboxylate.

Of the compounds of formula (Ia), another embodiment is the compounds of formula (Ia) wherein:
R¹ is —O—;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁵—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁵—C(═NR⁴)N(R⁴)R⁵, and —R⁵—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is the compounds of formula (Ia) wherein:
R¹ is —O—;
R^{2a} and R^{3a} are the same and selected from the group consisting of —R⁶—S—C(═NR⁴)N(R⁴)R⁵, —R⁶—O—C(═NR⁴)N(R⁴)R⁵, —R⁶—C(═NR⁴)N(R⁴)R⁵ and —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is the compounds of formula (Ia) wherein:
R¹ is —O—;
R^{2a} and R^{3a} are both —R⁶—S—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is the compounds of formula (Ia) wherein:
R¹ is —O—;
R^{2a} and R^{3a} are both —R⁶—S—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂ and —R⁶—N(R⁸)₂;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂ and —R⁶—N(R⁸)₂;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl.

One embodiment of the compounds of formula (Ia) is a compound selected from the group consisting of:
2-[2-(2-carbamimidoylsulfanylmethyl-phenoxy)-benzyl]-isothiourea;
2-(1-{2-[2-(1-carbamimidoylsulfanyl-ethyl)-phenoxy]-phenyl}-ethyl)-isothiourea;
2-[2-({[amino(imino)methyl]thio}methyl)-4-fluorophenoxy]-5-nitrobenzyl imidothiocarbamate;
2-[2-({[amino(imino)methyl]thio}methyl)-5-chlorophenoxy]-4-nitrobenzyl imidothiocarbamate;
2-[2-({[amino(imino)methyl]thio}methyl)-5-chlorophenoxy]-5-fluorobenzyl imidothiocarbamate;
2-[2-(2-carbamimidoylsulfanylmethyl-3-chlorophenoxy)-5-fluorobenzyl]isothiourea;
2-[2-(2-carbamimidoylsulfanylmethyl-4-chlorophenoxy)-5-fluorobenzyl]isothiourea;
2-[2-({[amino(imino)methyl]thio}methyl)-4-chlorophenoxy]benzyl imidothiocarbamate;
2-[2-({[amino(imino)methyl]thio}methyl)-3-chlorophenoxy]benzyl imidothiocarbamate;
2-[2-({[amino(imino)methyl]thio}methyl)-5-chlorophenoxy]benzyl imidothiocarbamate;
2-[2-({[amino(imino)methyl]thio}methyl)-5-chlorophenoxy]-5-nitrobenzyl imidothiocarbamate;
2-[2-({[amino(imino)methyl]thio}methyl)-4-chlorophenoxy]-5-nitrobenzyl imidothiocarbamate;
2-[2-(2-carbamimidoylsulfanylmethyl-5-fluorophenoxy)-5-fluorobenzyl]isothiourea;
2-[2-(2-carbamimidoylsulfanylmethyl-4-fluorophenoxy)-5-fluorobenzyl]isothiourea; and
2-[2-({[amino(imino)methyl]thio}methyl)-4-fluorophenoxy]benzyl imidothiocarbamate.

Of the compounds of formula (Ia), another embodiment is the compounds of formula (Ia) wherein:
R¹ is —S(O)ₚ— (where p is 0, 1 or 2);
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is the compounds of formula (Ia) wherein:
R¹ is —S(O)ₚ— (where p is 0, 1 or 2);
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

Of the compounds of formula (Ia), another embodiment is the compounds of formula (Ia) wherein:
R¹ is —S(O)ₚ— (where p is 0, 1 or 2);
R^{2a} and R^{3a} are both —R⁶—S—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is the compounds of formula (Ia) wherein:
R¹ is —S—;
R^{2a} and R^{3a} are both —R⁶—S—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

One embodiment of the compounds of formula (Ia) is a compound selected from the group consisting of:
2-[2-(2-carbamimidoylsulfanylmethyl-phenylsulfanyl)-benzyl]-isothiourea;
2-[2-(2-carbamimidoylsulfanylmethyl-4-fluoro-phenylsulfanyl)-5-fluoro-benzyl]-isothiourea;
2-[2-(2-carbamimidoylsulfanylmethyl-4-fluoro-phenylsulfanyl)-benzyl]-isothiourea;
2-[2-(2-carbamimidoylsulfanylmethyl-5-methyl-phenylsulfanyl)-benzyl]-isothiourea;
2-[2-(2-carbamimidoylsulfanylmethyl-4-methoxy-phenylsulfanyl)-benzyl]-isothiourea;
2-[2-(2-carbamimidoylsulfanylmethyl-5-methyl-phenylsulfanyl)-5-fluoro-benzyl]-isothiourea;
2-[2-(2-carbamimidoylsulfanylmethyl-4-methoxy-phenylsulfanyl)-5-fluoro-benzyl]-isothiourea;
2-[2-(2-carbamimidoylsulfanylmethyl-5-chloro-phenylsulfanyl)-5-fluoro-benzyl]-isothiourea;
2-[2-(2-carbamimidoylsulfanylmethyl-6-methylphenylsulfanyl)-benzyl]isothiourea;
2-[2-(2-carbamimidoylsulfanylmethyl-4,5-difluorophenylsulfanyl)-benzyl]isothiourea;
2-[2-(2-carbamimidoylsulfanylmethyl-4-methyl-phenylsulfanyl)-benzyl]-isothiourea;
2-[2-(2-carbamimidoylsulfanylmethyl-5-chloro-phenylsulfanyl)-benzyl]-isothiourea;
2-[2-(2-carbamimidoylsulfanylmethyl-5-fluoro-phenylsulfanyl)-benzyl]-isothiourea;
2-[2-(2-carbamimidoylsulfanylmethyl-5-fluoro-phenylsulfanyl)-5-fluorobenzyl]-isothiourea;
2-[2-(2-carbamimidoylsulfanylmethyl-4,5-difluorophenylsulfanyl)-5-fluorobenzyl]isothiourea;
2-{[2-({[amino(imino)methyl]thio}methyl)-4-fluorophenyl]thio}-3-nitrobenzyl imidothiocarbamate;
2-{[2-({[amino(imino)methyl]thio}methyl)-4-fluorophenyl]thio}-5-nitrobenzyl imidothiocarbamate;
2-[2-(2-carbamimidoylsulfanylmethyl-4-fluorophenylsulfanyl)-5-(trifluoromethyl)benzyl]isothiourea;
2-[2-(2-carbamimidoylsulfanylmethylphenylsulfanyl)-5-aminobenzyl]-isothiourea;
2-[2-(2-carbamimidoylsulfanylmethyl-4-chlorophenylsulfanyl)-5-fluorobenzyl]isothiourea;
2-[2-(2-carbamimidoylsulfanylmethylphenylsulfanyl)-5-ethylaminobenzyl]isothiourea;
2-[2-(2-carbamimidoylsulfanylmethyl-4-chlorophenylsulfanyl)-5-chlorobenzyl]isothiourea;
2-[2-(2-carbamimidoylsulfanylethyl-4-fluorophenylsulfanyl)-5-fluorobenzyl]isothiourea;
2-[2-(2-carbamimidoylsulfanylethyl-4-chlorophenylsulfanyl)-5-fluorobenzyl]isothiourea;
2-[2-(2-carbamimidoylsulfanylethylphenylsulfanyl)benzyl]isothiourea;
2-[2-(2-carbamimidoylsulfanylmethylphenylsulfanyl)-5-chlorobenzyl]-isothiourea;
2-[2-(2-carbamimidoylsulfanylmethylphenylsulfanyl)-5-(trifluoromethyl)benzyl]isothiourea;
2-[2-(2-methylcarbamimidoylsulfanylmethylphenylsulfanyl)benzyl]-methylisothiourea;
2-[2-(2-carbamimidoylsulfanylmethyl-4-fluorophenylsulfanyl)-5-(methylsulfonyl)benzyl]isothiourea;
2-({2-({[amino(imino)methyl]thio}methyl)-4-[(dimethylamino)sulfonyl]phenyl}thio)-5-fluorobenzyl imidothiocarbamate;
2-[2-(2-carbamimidoylsulfanylmethylphenylsulfanyl)benzyl]-isothiourea;
2-{[2-({[amino(imino)methyl]thio}methyl)-4-fluorophenyl]thio}-4-(methylsulfonyl)benzyl imidothiocarbamate; and
2-{[2-({[amino(imino)methyl]thio}methyl)-4-chlorophenyl]thio}-5-cyanobenzyl imidothiocarbamate.

Of the compounds of formula (Ia), another embodiment is the compounds of formula (Ia) wherein:

R¹ is —S(O)₂—;
R^{2a} and R^{3a} are both —R⁶—S—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

One embodiment of the compounds of formula (Ia) is a compound selected from the group consisting of:
2-[2-(2-carbamimidoylsulfanylmethylphenylsulfonyl)benzyl]-isothiourea; and
2-[2-(2-carbamimidoylsulfanylmethyl-4-fluorophenylsulfonyl)-5-fluorobenzyl]isothiourea.

Of the compounds of formula (Ia), another embodiment is the compounds of formula (Ia) wherein:
R¹ is —S(O)ₚ— (where p is 0, 1 or 2);
R^{2a} and R^{3a} are both —R⁶—S—C(═NR⁴)N(R⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂—R⁶—N(R⁸)₂—R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is the compounds of formula (Ia) wherein:
R¹ is —S—;
R^{2a} and R^{3a} are both —R⁶—S—C(═NR⁴)N(R⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂—R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is 2-(6-((aminoamidino)thiomethyl)phenyl)thio-1-((aminoamidino)thiomethyl)benzene.

Of the compounds of formula (Ia), another embodiment is the compounds of formula (Ia) wherein:
R¹ is —S(O)ₚ— (where p is 0, 1 or 2);
R^{2a} and R^{3a} are both —R⁶—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂—R⁶—N(R⁸)₂—R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is the compounds of formula (Ia) wherein:
R¹ is —S—;
R^{2a} and R^{3a} are both —R⁶—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

One embodiment of the compounds of formula (Ia) is a compound selected from the group consisting of:
2-(6-(2-amidinoethyl)phenyl)thio-1-(2-amidinoethyl)benzene;
2-(6-(amidinomethyl)phenyl)thio-1-(amidinomethyl)benzene; and
2-(6-(3-amidinopropyl)phenyl)thio-1-(3-amidinopropyl)benzene.

Of the compounds of formula (Ia), another embodiment is the compounds of formula (Ia) wherein:
R¹ is —S(O)ₚ— (where p is 0, 1 or 2);
R^{2a} and R^{3a} are both —R⁶—C(═NCN)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)N(R⁸)₂, wherein each t is independently 1 or 2 and each p is 0, 1 or 2;
R^{3b}, R^{3c}, R_{3d} and R^{3e} are each independently selected from the group consisting of hydrogen, alkyl, halo, haloalkyl, —R⁶—OR⁷, —R⁶—CN, —R⁶—NO₂—R⁶—N(R⁸)₂—R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is the compounds of formula (Ia) wherein:
R¹ is —S—;
R^{2a} and R^{3a} are both —R⁶—C(═NCN)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂—R⁶—N(R⁸)₂—R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

One embodiment of the compounds of formula (Ia) is a compound selected from the group consisting of:
2-(6-((cyanoamidino)methyl)phenyl)thio-1-((cyanoamidino)methyl)benzene;
2-(6-(2-(cyanoamidino)ethyl)phenyl)thio-1-(2-(cyanoamidino)ethyl)benzene; and
2-(6-(3-(cyanoamidino)propyl)phenyl)thio-1-(3-(cyanoamidino)propyl)benzene.

Of the compounds of formula (Ia), another embodiment is the compounds of formula (Ia) wherein:
R¹ is —S(O)ₚ— (where p is 0, 1 or 2);
R^{2a} and R^{3a} are both —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is the compounds of formula (Ia) wherein:
R¹ is —S—;
R^{2a} and R^{3a} are both —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is 1-(2-(2-(guanidinomethyl)phenylthio)benzyl)guanidine.

Of the compounds of formula (Ia), another embodiment is the compounds of formula (Ia) wherein:
R¹ is —S(O)ₚ— (where p is 0, 1 or 2);
R^{2a} and R^{3a} are both —R⁶—C(O)—N═C[N(R⁴)(R⁵)]N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is the compounds of formula (Ia) wherein:
R¹ is —S—;
R^{2a} and R^{3a} are both —R⁶—C(O)—N═C[N(R⁴)(R⁵)]N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is 2,2′-thiobis(N-(diaminomethylene)benzamide).

Of the compounds of formula (Ia), another embodiment is the compounds of formula (Ia) wherein:
R¹ is —C(O)—;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is the compounds of formula (Ia) wherein:
R¹ is —C(O)—;
R^{2a} and R^{3a} are each —R⁶—S—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

One embodiment of the compounds of formula (Ia) is a compound selected from the group consisting of:
2-(6-(amidinothiomethyl)phenyl)carbonyl-1-(amidinothiomethyl)benzene;
2-(6-(amidinothiomethyl)phenyl)carbonyl-1-(amidinothiomethyl)-5-fluorobenzene;
2-(6-(amidinothiomethyl)phenyl)carbonyl-1-(amidinothiomethyl)-4-fluorobenzene;
2-(6-(amidinothiomethyl)phenyl)carbonyl-1-(amidinothiomethyl)-4-chlorobenzene; and
2-(6-(amidinothiomethyl)phenyl)carbonyl-1-(amidinothiomethyl)-5-chlorobenzene.

Of the compounds of formula (Ia), another embodiment is the compounds of formula (Ia) wherein:
R¹ is —C(O)—;
R^{2c} and R^{3c} are each —R⁶—S—C(═NR⁴)N(R⁴)R⁵;
R^{2a}, R^{2b}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3a}, R^{3b}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

One embodiment of the compounds of formula (Ia) is 4,4-diisothiourea benzophenone.

Of the compounds of formula (Ia), another embodiment is the compounds of formula (Ia) wherein:
R¹ is —N(R⁴)—;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ia), another embodiment is the compounds of formula (Ia) wherein:
R¹ is —N(R⁴)—;
R^{2a} and R^{3a} are each —R⁶—S—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

One embodiment of the compounds of formula (Ia) is 2,2-(methylazanediyl)bis(2,1-phenylene)bis(methylene)dicarbamimidothioate.

Of the compounds of formula (I) as described above in the Summary of the Invention, another embodiment is the compounds of formula (Ib):

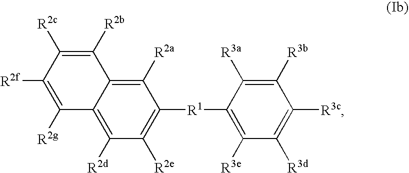

wherein:
R¹ is a direct bond, —O—, —S(O)ₚ— (where p is 0, 1 or 2), —C(O)— or —N(R⁴)—;
R^{2a}, R^{2b}, R^{2c}, R^{2d}, R^{2e}, R^{2f} and R^{2g} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2 and wherein at least one of R²,
R^{2b}, R^{2c}, R^{2d}, R^{2e}, R^{2f} and R^{2g} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{3a}, R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—C(═NR⁴)N(R⁴)R⁵, —R⁶—C(═NR⁴)N(R⁴)R⁵, and —R⁶—N(R⁷)—C(═NR⁴)N(R⁴)R⁵ wherein each t is independently 1 or 2 and each p is 0, 1 or 2 and wherein at least one of R³,
R^{3b}, R^{3c}, R^{3d} and R^{3e} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ib), one embodiment is the compounds of formula (Ib) wherein:
R¹ is —S(O)ₚ— (where p is 0, 1 or 2);
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d}, R^{2e}, R^{2f} and R^{2g} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ib), another embodiment is the compounds of formula (Ib) wherein:
R¹ is —S(O)ₚ— (where p is 0, 1 or 2);
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d}, R^{2e}, R^{2f} and R^{2g} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ib), another embodiment is the compounds of formula (Ib) wherein:
R¹ is —S(O)ₚ— (where p is 0, 1 or 2);
R^{2a} and R^{3a} are both —R⁶—S—C(═NR⁴)N(R⁴)R⁵;
R^{2b}, R^{2c}, R^{2d}, R^{2e}, R^{2f} and R^{2g} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ib), another embodiment is the compounds of formula (Ib) wherein:
R¹ is —S;
R^{2a} and R^{3a} are both —R⁶—S—C(═NR⁴)N(R⁴)R⁵;
R^{2b}, R^{2c}, R^{2d}, R^{2e}, R^{2f} and R^{2g} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ib), another embodiment is a compound selected from the group consisting of:
2-[2-(1-carbamimidoylsulfanylmethyl-naphthalen-2-ylsulfanyl)-benzyl]-isothiourea; and
2-[2-(1-carbamimidoylsulfanylmethylnaphthalen-2-ylsulfanyl)-5-fluorobenzyl]-isothiourea.

Of the compounds of formula (I) as described above in the Summary of the Invention, another embodiment is the compounds of formula (Ic):

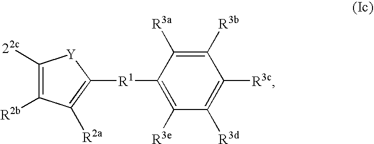

wherein:
Y is —O—, —S— or —N(R⁴)—;
R¹ is a direct bond, —O—, —S(O)ₚ— (where p is 0, 1 or 2), —C(O)— or —N(R⁴)—;
R^{2a}, R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2 and wherein at least one of R^{2a}, R^{2b} and R^{2c} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{3a}, R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂—R⁶—N(R⁸)₂—R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵ wherein each t is independently 1 or 2 and each p is 0, 1 or 2 and wherein at least one of R^{3a},
R^{3b}, R^{3c}, R^{3d} and R^{3e} is independently selected from the group consisting of —R⁶—S—C(═NR⁴)N(R⁴)R⁵, —R⁶—S—C(═NR⁴)N(R⁴)N(R⁴)R⁵, —R⁶—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ic), one embodiment is the compounds of formula (Ic) wherein:
Y is —S—;
R¹ is a direct bond, —O—, —S(O)ₚ— (where p is 0, 1 or 2), —C(O)— or —N(R⁴)—;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁸—NO₂—R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ic), another embodiment is the compounds of formula (Ic) wherein:
Y is —S—;
R¹ is a direct bond, —O—, —S(O)ₚ— (where p is 0, 1 or 2), —C(O)— or —N(R⁴)—;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂—R⁶—N(R⁸)₂—R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ic), another embodiment is the compounds of formula (Ic) wherein:
Y is —S—;
R¹ is —S(O)ₚ— (where p is 0, 1 or 2);
R^{2a} and R^{3a} are both —R⁶—S—C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ic), another embodiment is the compounds of formula (Ic) wherein:
Y is —S—;
R¹ is —S—;
R^{2a} and R^{3a} are both —R⁶—S—C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Ic), another embodiment is (2-{[2-({[amino(imino)methyl]thio}methyl)-4-fluorophenyl]thio}-3-thienyl)methyl imidothiocarbamate.

Of the compounds of formula (I) as described above in the Summary of the Invention, another embodiment is the compounds of formula (Id):

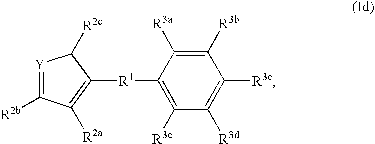

wherein:
Y is —O—, —S— or —N(R⁴)—;
R¹ is a direct bond, —O—, —S(O)ₚ— (where p is 0, 1 or 2), —C(O)— or —N(R⁴)—;
R^{2a}, R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2 and wherein at least one of R^{2a}, R^{2b} and R^{2c} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{3a}, R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵ wherein each t is independently 1 or 2 and each p is 0, 1 or 2 and wherein at least one of R^{3a}, R^{3b}, R^{3c}, R^{3d} and R^{3e} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Id), one embodiment is the compounds of formula (Id) wherein:
Y is —S—;
R¹ is a direct bond, —O—, —S(O)ₚ— (where p is 0, 1 or 2), —C(O)— or —N(R⁴)—;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂—R⁶—N(R⁸)₂—R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Id), another embodiment is the compounds of formula (Id) wherein:
Y is —S—;
R¹ is a direct bond, —O—, —S(O)ₚ— (where p is 0, 1 or 2), —C(O)— or —N(R⁴)—;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Id), another embodiment is the compounds of formula (Id) wherein:
Y is —S—;
R¹ is —S(O)ₚ— (where p is 0, 1 or 2);
R^{2a} and R^{3a} are both —R⁶—S—C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Id), another embodiment is the compounds of formula (Id) wherein:
Y is —S—;
R¹ is —S—;
R^{2a} and R^{3a} are both —R⁶—S—C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Of the compounds of formula (Id), another embodiment is (4-{[2-({[amino(imino)methyl]thio}methyl)-4-fluorophenyl]thio}-3-thienyl)methyl imidothiocarbamate.

Another aspect of this invention are pharmaceutical compositions comprising a pharmaceutically acceptable excipient and a therapeutically effective amount of a compound of the invention, as set forth above in the Summary of the Invention, as a stereoisomer, enantiomer, tautomer thereof or mixtures thereof, or a pharmaceutically acceptable salt, solvate or prodrug thereof.

One embodiment of this aspect of the invention are pharmaceutical compositions comprising a pharmaceutically acceptable excipient and a therapeutically effective amount of an embodiment of a compound of formula (I), as set forth above, as a stereoisomer, enantiomer, tautomer thereof or mixtures thereof, or a pharmaceutically acceptable salt, solvate or prodrug thereof.

Another embodiment of this aspect of the invention are pharmaceutical compositions comprising a pharmaceutically acceptable excipient and a therapeutically effective amount of an embodiment of a compound of formula (II), as set forth above, as a stereoisomer, enantiomer, tautomer thereof or mixtures thereof, or a pharmaceutically acceptable salt, solvate or prodrug thereof.

Another aspect of the invention are methods for treating an iron disorder in a mammal, preferably a human, or a disease or condition associated with an iron disorder in a mammal, preferably a human, wherein the method comprises administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising an embodiment of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

One embodiment of this aspect is where the disease or condition associated with the iron disorder is due to an accumulation of iron in the body tissues of the mammal.

Another embodiment of this aspect is where the iron disorder is a primary iron overload disorder.

Of this embodiment, a preferred embodiment is where the primary iron overload disorder is independently selected from the group consisting of hereditary hemochromatosis, juvenile hemochromatosis, ferroportin disease, neonatal hemochromatosis, Bantu siderosis, African iron overload, gracile syndrome, ataxia, and Friedreich Ataxia. A more preferred embodiment is where the primary iron overload is hereditary hemochromatosis.

Another embodiment of this aspect is where the iron disorder is a secondary iron overload disorder.

Another embodiment of this aspect is where the iron disorder is transfusional iron overload disorder.

Another embodiment of this aspect is where the disease or condition is independently selected from the group consisting of thalassemia (beta and alpha, major, minor and intermedia), hypochromic microcytic anemia, sickle cell anemia, microcytic iron loading anemia, hereditary sideroblastic anemia, congenital dyserythropoeitic anemia, porphyria cutanea tarda, pyruvate kinase deficiency, hereditary atransferrinemia, ceruloplasmin deficiency, myelodysplastic syndromes, pulmonary hemosiderosis, aceruloplasminemia and x-linked sideroblastic anemia.

Another embodiment of this aspect is where the disease or condition associated with an iron overload is independently selected from the group consisting of neurodegenerative disease (including ALS, prion diseases, Parkinson's, and Alzheimers), cardiovascular disease (including atherosclerosis, ischemic cerebrovascular disease and ischemic stroke), inflammation (including arthritis and disease progression in viral hepatitis), cancer, insulin resistance, non-alcoholic liver disease, alcoholic liver disease, and infectious disease (including HIV, malaria and Yersinia infections).

Another embodiment of the invention are methods for treating an iron disorder associated with DMT1 activity in a mammal, preferably a human, or for treating a disease or condition associated with DMT1 activity in a mammal, preferably a human, wherein the method comprises administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising an embodiment of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

Of this embodiment, one embodiment is where the DMT1 activity is upregulated (i.e., increased levels of DMT1 activity as compared to normal levels of DMT1 activity).

Of this embodiment, another embodiment is where the therapeutically effective amount administered to the mammal is a DMT1-inhibitory amount.

Specific embodiments of the compounds of the invention are described in more detail below in the following sections.

### Utility and Testing of the Compounds of the Invention

The present invention is directed to compounds and pharmaceutical compositions comprising the compounds, as described herein and above in the Summary of the Invention, which are useful in the treatment of iron disorders in a mammal, preferably a human, by modulating, preferably inhibiting, DMT1 activity.

The term “iron disorder” refers to a condition in a mammal, preferably a human, wherein the level of iron in the body is outside the normal range for the particular mammal (i.e. abnormal iron level), such as an elevated or a decreased iron serum level compared to the normal iron serum level for the mammal or an increased or decreased level of iron in the liver of the mammal as compared to the normal level of iron in the liver in the mammal. Abnormal iron serum levels can be determined by direct measurement of serum iron using a colorimetric assay, or by the standard transferrin saturation assay (which reveals how much iron is bound to the protein that carries iron in the blood), or by the standard serum ferritin assay. For example, transferrin saturation levels of 45% or higher are usually indicative of abnormally high levels of iron in the serum. Abnormal iron levels in the liver can be determined measuring the iron content of the liver from tissue obtained by a liver biopsy or by imaging technique such as MRI and/or SQUID. The degree of iron levels in other tissues (e.g., brain, heart) may also be estimated using these and other imaging techniques. Preferably, for purposes of this invention, an abnormal iron level is an elevated iron level in serum or tissue.

The term “iron disorders” therefore includes both iron deficiency disorders and iron overload disorders. Preferably, the iron disorder is an iron overload disorder, such as primary iron overload disorder (including, but not limited to, hereditary hemochromatosis, juvenile hemochromatosis, ferroportin disease, neonatal hemochromatosis, Bantu siderosis, African iron overload, gracile syndrome, ataxia, and Friedreich Ataxia, as well as all of the anemias listed below in which patients may not be transfused but may become iron overloaded due to increased erythroid drive and the resulting increased iron absorption in the gut) and secondary (or transfusional) iron overload disorder which can be caused by repeated transfusions used to treat a number of distinct anemias, including, but not limited to, thalassemia (beta and alpha, major, minor and intermedia), hypochromic microcytic anemias, sickle cell anemia, microcytic iron loading anemias, hereditary sideroblastic anemias, congenital dyserythropoeitic anemias, porphyria cutanea tarda, pyruvate kinase deficiency, hereditary atransferrinemia, ceruloplasmin deficiency, myelodysplastic syndromes, pulmonary hemosiderosis, aceruloplasminemia and x-linked sideroblastic anemia.

Iron disorders of particular interest in the practice of the invention are iron overload disorders where the level of iron in a mammal is higher than the normal level of iron in the mammal. Such iron overload disorders including, but are not limited to, primary iron overload disorders (including, but not limited to, hereditary hemochromatosis, juvenile hemochromatosis, ferroportin disease, neonatal hemochromatosis, Bantu siderosis, African iron overload, gracile syndrome, ataxia, and Friedreich Ataxia, as well as all of the anemias listed below, in which patients may not be transfused but may become iron overloaded due to increased erythroid drive and the resulting increased iron absorption in the gut), and secondary (transfusional) iron overload disorders (including, but not limited to, thalassemia (beta and alpha, major, minor and intermedia)), hypochromic microcytic anemias, sickle cell anemia, microcytic iron loading anemias, hereditary sideroblastic anemias, congenital dyserythropoeitic anemias, porphyria cutanea tarda, pyruvate kinase deficiency, hereditary atransferrinemia, ceruloplasmin deficiency, myelodysplastic syndromes, pulmonary hemosiderosis, aceruloplasminemia, and x-linked sideroblastic anemia. Iron overload may also be responsible for a portion of the pathology observed in neurodegenerative diseases (including ALS, prion diseases, Parkinson's, Alzheimers), cardiovascular diseases (including atherosclerosis, ischemic cerebrovascular disease and ischemic stroke), inflammatory diseases and conditions (including arthritis and disease progression in viral hepatitis), cancer, insulin resistance, non-alcoholic liver disease, alcoholic liver disease, and infectious disease (including HIV, malaria and Yersinia infections).

The compounds of the invention, and pharmaceutical compositions comprising the compounds of the invention, are useful in treating iron disorders by modulating, preferably inhibiting, DMT1 activity. There is evidence that the upregulation (i.e., increased activity) of DMT1 has a role in iron disorders caused by genetic abnormalities, such as hereditary hemochromatosis. Hereditary hemochromatosis is an iron overload disorder due to intestinal iron hyperabsorption. Hereditary hemochromatosis is characterized by a slow accumulation of iron from the diet to toxic levels resulting in tissue injury and multi-organ malfunction. Patients, typically men, develop symptoms of hemochromatosis in their fourth and fifth decade with variable combinations of cirrhosis, hepatoma, arthritis, hypogonadism, diabetes mellitus and cardiomyopathy. The biochemical profile shows elevated transferrin saturation above 45% and a high serum ferritin. The underlying genetic defect in hereditary hemochromatosis is a mutation in the hemochromatosis gene (HFE) on chromosome 6p21. 90% of Northern Europeans with hereditary hemochromatosis are homozygous for a single missense mutation, C282Y in exon 4 of the HFE gene.

DMT1 activity has also been implicated in the etiology and pathophysiology of hypochromic microcytic anemias, thalassemia, microcytic iron loading anemias, hereditary sideroblastic anemias, hereditary hypochromic anemias, congenital dyserythropoietic anemias, pyruvate kinase deficiency, hereditary atransferrinemia, and certain myelodysplastic syndromes, as there is a direct correlation between the degree of iron limited anemia, increased DMT1 expression in the duodenum and, by extension, increased iron absorption via DMT1 (Morgan et al., Blood Cells Molecules and Diseases, 2002, 29:384-399).

There is also evidence that DMT1 has a role in iron disorders such as acquired iron overload. The risk factors for acquired iron overload might include for example excessive ingestion of red meat, iron supplements or foods that are iron fortified. Acquired iron overload can also occur from the use of iron cookware, drinking unpurified tap water, use of oral contraceptives, blood transfusions and cigarette smoking. DMT1 pattern of expression and function supports it as a candidate target for the treatment of acquired iron overload and other related maladies.

In addition to the small intestine, DMT1 is also highly expressed in the kidney suggesting a role in renal iron handling and possibly reabsorption of filtered iron (Ferguson et al., Am. J. Physiol. Renal. Physiol., 2001, 280: F803-F814) and is also involved in the delivery of iron to peripheral tissues by transferrin (Fleming et al., Proc. Natl. Acad. Sci., 1998, 85:1148-1153). DMT1 inhibitors, when dosed in a fashion that increases their systemic exposure, may be useful in an acute unloading of iron via the urine, by inhibiting DMT1 expressed in the kidney.

DMT1 may also play a role in regulating iron flux to the brain. As there is some indication that iron overload in the brain may play a role in brain pathology, such as Alzheimer's, DMT1 inhibitors may act to reduce the amount of iron absorbed by the brain, when dosed in a fashion that increases their systemic exposure and allows them to play a role at the blood brain barrier or within the brain (Lehmann et al., 2006, J. Med. Genet., 2006, 43(10):e52; Schenck et al., Top. Magn Reson. Imaging., 2006, 17(1):41-50).

Studies show that mutant mice that are defective in DMT1 activity (mk/mk) develop hyprochromic microcytic anemia, a severe form of iron deficiency anemia, due to a defect in intestinal iron absorption. In contrast, the hfe^{-/-} knockout mouse model of hereditary hemochromatosis is characterized by an enhanced intestinal iron uptake and total body iron overload. The hfe^{-/-}:mk/mk double mutant mouse, which carries mutations in both the HFE and DMT1 genes, fails to load iron, indicating that hemochromatosis (hfe-1) can be prevented by blocking the flux of iron through the DMT1 protein (Levy et al., J. Clin. Invest., 2000, 105:1209-16). In addition, studies of human patients with hereditary hemochromatosis show that DMT1 is inappropriately upregulated at the intestinal brush border. This aberrant excessive expression of DMT1 in hereditary hemochromatosis is fundamental to the primary pathophysiology of this condition (Zoller et al., Gastroenterology, 2001, 120:1412-1419). These findings have made DMT1 a therapeutic target for the treatment of iron overload disorders in general, and, in particular, for the treatment of hereditary hemochromatosis. In further support of DMT1 as a therapeutic target in the treatment of iron overload, it has been shown in clinical studies that the majority of the excess iron burden is absorbed in the form of ferrous (non-heme) iron, as opposed to heme-iron (Lynch et al., Blood, 1989, 74:2187-2193).

While not wishing to be bound to any particular mechanism of action, the compounds of the invention, and pharmaceutical compositions comprising the compounds of the invention, are useful in treating iron disorders by directly interacting with a region of the DMT1 protein that modulates or controls iron flux. A direct interaction is supported by the fact that the compounds are not potent inhibitors of cation flux in the closely related transporter Natural Resistance-Associated Macrophage Protein-1 (NRAMP1). In general, the compounds of the invention modulate the activity of DMT1 downwards, thereby inhibiting the ability of DMT1 to uptake non-heme iron across the cellular membrane. The compounds of the invention are therefore considered to be DMT1 inhibitors and are therefore useful in treating iron disorders which are ameliorated by the modulation, preferably the inhibition, of DMT1 activity. The compounds of the invention, as DMT1 inhibitors, are also useful in reducing normal or slightly abnormal iron serum levels in a mammal, preferably a human, wherein the reduction of iron serum levels provides a therapeutic benefit to the mammal, preferably a human, such as neuroprotective activity after a stroke.

The compounds of the invention, and pharmaceutical compositions comprising the compounds of the invention, are also useful in treating or preventing symptoms, diseases and/or conditions in a mammal associated with hereditary hemochromatosis due to accumulation of iron in body tissues such as arthritis, liver disease, heart disease, impotence, early menopause, abnormal skin pigmentation, thyroid deficiency, damage to pancreas, diabetes, and damage to adrenal gland (Sheth et al., Annu. Rev. Med., 2000, 51:443-464).

The compounds of the invention, and pharmaceutical compositions comprising the compounds of the invention, are also useful in treating or preventing other forms of hemochromatosis including, but are not limited to, juvenile hemochromatosis and neonatal hemochromatosis. Juvenile hemochromatosis has a much earlier onset and exhibits more severe symptoms such as endocrine dysfunction, joint disease, and cardiac abnormalities due to excessive iron deposition from an early age. Neonatal hemochromatosis is a rare fetal gestational condition that results in iron accumulation in the liver of the fetus.

The compounds of the invention, and pharmaceutical compositions comprising the compounds of the invention, are also useful in treating or preventing transfusional iron overload. Chronic blood transfusion is the established therapy for thalassaemia major, bone marrow failure and complications of sickle cell anaemia and other related disorders. With hypertransfusion, the systemic iron load accumulates. Because there is no natural way for the body to eliminate the iron, the excess iron in the transfused blood builds up to cause iron overload and becomes toxic to tissues and organs, particularly the liver, heart, and pancreas. Transfusional iron overload typically results in the patient's premature death from organ failure. The transfusional iron overload is unfortunately augmented by increased iron absorption, which is the natural attempt of the body to increase iron levels in order to promote erythropoiesis, which is itself compromised by the disease states above. Decreased absorption of iron by the inhibition of DMT1 activity may reduce the iron overload related to the transfusional iron overload and supports the use of DMT1 inhibitors for the treatment of this disease.

In addition, due to iron's ability to generate reactive oxygen species (free radicals), which can result in inflammation and tissue damage, the compounds of the invention, and pharmaceutical compositions comprising the compounds of the invention, may also be useful as anti-inflammatory or neuroprotective agents due to their ability to reduce iron serum levels by the modulation, preferably inhibition, of DMT1 activity.

The general value of the compounds of the invention, and pharmaceutical compositions comprising the compounds of the invention, in modulating, preferably inhibiting, DMT1 activity can be determined using the assays described herein or below in the Biological Assays section. Alternatively, the general value of the compounds of the invention, and pharmaceutical compositions comprising the compounds of the invention, in treating iron disorders in humans may be established in industry standard animal models for demonstrating the efficacy of compounds in treating iron disorders.

In particular, identification of the compounds of the invention ability to modulate, preferably to inhibit, DMT1 activity, can be assessed using a variety of in vitro and in vivo assays, for measuring uptake of reduced iron (Fe²⁺). One such protocol involves the screening of chemical agents for ability to modulate the activity of DMT1 thereby identifying it as a modulating agent. The in vitro activity of DMT1 can be measured in cell based assays by either directly measuring iron flux (using a radioactively labelled iron ⁵⁵Fe) or by measuring the fluorescence of a cell permeable iron fluorophore such as calcein. Stable cell lines overexpressing DMT1 are exposed to ⁵⁵Fe or loaded with calcein and then compound is applied. Decreased flux of ⁵⁵Fe or lack of fluorescence quenching indicates that the given modulator has inhibited DMT1 function (Picard et al., J. Biol. Chem., 2000, 275(46):35738-45 and Wetli et al., Chem. Biol. 2006 September; 13(9):965-72). Alternatively, in another format electrophysiological techniques can be used to measure the current or iron or other metals traversing the cell membrane with DMT1 in a Xenopus oocyte or other cell based system (Gunshin et al., Nature, 1997, 31; 388(6641):482-8).

Other assays may involve intestinal cells or tissues which express endogenous DMT1, using the same detection techniques such as fluorescence, radiolabelled iron or electrophysiology. A human Caco2 cell line can be used for such assays (Alvarez-Hernandez et al., Biochimica. et Biophysica. Acta., 1991, 1070:205-208). These assays can be performed in the presence of desferroxamine to render the cells iron deficient and upregulate DMT1 expression. Alternatively, intestinal tissue may be used, either as gut rings which will take up iron (Raja et al., Cell. Biochemistry and Function, 1987, 5:69-76; Leppert et al., J. of Pharm. Sci., 1994, 83:976-981), or as gut slices ex vivo (Vaghefi et al., Reprod. Nutr. Dev., 1998, 38:559-566) where iron flux across the epithelial layer can be assessed in an Ussing chamber. In these assays, tissue can be excised from iron replete or iron deficient animals. In addition, the heme versus non-heme iron absorptive capacity of the tissue can be measured.

These assays can be carried out in transfected cells, or cell or tissue endogenously expressing the channel of interest in a natural endogenous setting or in a recombinant setting. Other methods of testing the compounds disclosed herein are also readily known and available to those skilled in the art.

Compounds of the invention can also be tested in a variety of in vivo models so as to determine if they alleviate a particular iron disorder in a mammal, particularly an iron overload disorder, with minimal adverse events. The assays described herein and below in the Biological Assays Section are useful in assessing the in vivo activity of the compounds of the invention.

For example, a typical rat model of iron overload disorder can be created by establishing an iron deficient state in the rate, which will then cause the upregulation of DMT1 expression and activity, resulting in increased iron absorption. These models can be used to demonstrate that compounds of the invention have the ability to modulate, preferably inhibit, the activity of DMT1 as demonstrated by the increase in serum iron levels in the iron-deficient rat. Iron deficiency is induced in these rat models in order to mimic the DMT1 over-expression and iron hyperabsorption observed in humans having iron overload disorders such as hereditary hemochromatosis as well as humans suffering from thalassemia.

Alternatively, an iron deficient, and therefore hyperabsorptive state, may be induced by dietary means, such as, for example, treatment with phenylhydrazine, or by phlebotomy (Refino et al., Am. J. Clin. Nutr. 1983, 37:904-909; Redondo et al., Lab. Animal Sci. 1995, 45:578-583; Frazer et al., Gastroenterology, 2002, 123:835-844). Alternatively, iron absorption can also be stimulated by creating an hypoxic state to stimulate erythropoiesis (Raja et al., Br. J. Haematol., 1988, 68:373-378). In these models, a compound's efficacy can be assessed by measuring reduced iron flux via the duodenum acutely or by monitoring whether chronic exposure to a compound causes a decrease in the amount of iron loading as measured by serum iron, transferrin saturation, ferritin and liver iron. Alternatively, iron flux in these animals can be measured by tracing the absorption of radioactive iron administered orally. These experiments can also be performed in iron replete animals, although changes in these parameters will be less pronounced and therefore compound efficacy will be more difficult to judge.

Genetic rat models of iron overload offers another format to show efficacy of DMT1 inhibitors in preventing further iron loading. These models are applicable to a variety of iron disorders such as hereditary hemochromatosis (Levy et al., Blood, 1999, 94:9-11), juvenile hemochromatosis (Huang et al., J. Clin. Invest., 2005 115:2187-2191), beta-2-microglobulin (de Sousa et al., Immun. Lett., 1994, 39:105-111), thalassemia (Ciavatta et al., Proc. Nat. Acad. Sci., 1995, 92: 9259-9263), hypotransferrinmia (Craven et al., Proc. Nat. Acad. Sci., 1987, 84(10):3457-61) and other hypochromic microcytic anemias. A compound's efficacy can be assessed by measuring reduced iron flux via the duodenum acutely or by monitoring whether chronic exposure to a compound causes a decrease in the amount of iron loading as judged by serum iron, transferrin saturation, ferritin and liver iron. Alternatively, iron flux in these animals can be measured by tracing the absorption of radioactive iron administered orally.

Typically, a successful therapeutic agent of the present invention will meet some or all of the following criteria. Oral availability should be at less than 5%. Animal model efficacy is less than about 0.1 μg to about 100 mg/Kg body weight and the target human dose is between 0.1 μg to about 100 mg/Kg body weight, although doses outside of this range may be acceptable (“mg/Kg” means milligrams of compound per kilogram of body mass of the subject to whom it is being administered). The therapeutic index (or ratio of toxic dose to therapeutic dose) should be greater than 100. The potency (as expressed by IC₅₀ value) should be less than 10 μM, preferably below 1 μM and most preferably below 50 nM. The IC₅₀ (“Inhibitory Concentration—50%”) is a measure of the amount of compound required to achieve 50% inhibition of DMT1, over a specific time period, in an assay of the invention.

In another use of the invention, the compounds of the invention can be used in in vitro or in vivo studies as exemplary agents for comparative purposes to find other compounds useful in the treatment of an iron disorder or diseases or conditions associated with an iron disorder.

In another use of the invention, the compounds of the invention can be used in the preparation of a medicament for the treatment of an iron disorder in a mammal or for the treatment of a disease or condition associated with an iron disorder in a mammal.

### Pharmaceutical Compositions of the Invention and Administration

The present invention also relates to pharmaceutical composition containing the compounds of the invention disclosed herein. In one embodiment, the present invention relates to a composition comprising compounds of the invention in a pharmaceutically acceptable carrier, excipient or diluent and in an amount effective to modulate, preferably inhibit, DMT1 in order to treat iron disorders when administered to an animal, preferably a mammal, most preferably a human patient.

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in REMINGTON'S PHARMACEUTICAL SCIENCES (Mack Pub. Co., N.J. current edition).

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1 and about 70% of the weight of the composition. Preferred oral pharmaceutical compositions contain between about 4% and about 50% of the compound of the invention. Preferred pharmaceutical compositions and preparations according to the present invention are prepared so that a parenteral dosage unit contains between 0.01 to 10% by weight of the compound prior to dilution of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 10% w/v (weight per unit volume).

The pharmaceutical composition of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 Kg mammal) from about 0.001 mg/Kg (i.e., 0.07 mg) to about 100 mg/Kg (i.e., 7.0 g); preferably a therapeutically effective dose is (for a 70 Kg mammal) from about 0.01 mg/Kg (i.e., 0.7 mg) to about 50 mg/Kg (i.e., 3.5 g); more preferably a therapeutically effective dose is (for a 70 Kg mammal) from about 1 mg/Kg (i.e., 70 mg) to about 25 mg/Kg (i.e., 1.75 g).

The ranges of effective doses provided herein are not intended to be limiting and represent preferred dose ranges. However, the most preferred dosage will be tailored to the individual subject, as is understood and determinable by one skilled in the relevant arts. (see, e.g., Berkow et al., eds., The Merck Manual, 16^{th} edition, Merck and Co., Rahway, N.J., 1992; Goodman et al., eds., Goodman and Gilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18^{th} edition, Mack Publishing Co., Easton, Pa. (1990); Katzung, Basic and Clinical Pharmacology, Appleton and Lange, Norwalk, Conn. (1992)).

The total dose required for each treatment can be administered by multiple doses or in a single dose over the course of the day, if desired. Generally, treatment is initiated with smaller dosages, which are less than the optimum dose of the compound. Thereafter, the dosage is increased by small increments until the optimum effect under the circumstances is reached. The diagnostic pharmaceutical compound or composition can be administered alone or in conjunction with other diagnostics and/or pharmaceuticals directed to the pathology, or directed to other symptoms of the pathology. The recipients of administration of compounds and/or compositions of the invention can be any vertebrate animal, such as mammals. Among mammals, the preferred recipients are mammals of the Orders Primate (including humans, apes and monkeys), Arteriodactyla (including horses, goats, cows, sheep, pigs), Rodenta (including mice, rats, rabbits, and hamsters), and Carnivora (including cats, and dogs). Among birds, the preferred recipients are turkeys, chickens and other members of the same order. The most preferred recipients are humans.

For topical applications, it is preferred to administer an effective amount of a pharmaceutical composition according to the invention to target area, e.g., skin surfaces, mucous membranes, and the like, which are adjacent to peripheral neurons which are to be treated. This amount will generally range from about 0.0001 mg to about 1 g of a compound of the invention per application, depending upon the area to be treated, whether the use is diagnostic, prophylactic or therapeutic, the severity of the symptoms, and the nature of the topical vehicle employed. A preferred topical preparation is an ointment, wherein about 0.001 to about 50 mg of active ingredient is used per cc of ointment base. The pharmaceutical composition can be formulated as transdermal compositions or transdermal delivery devices (“patches”). Such compositions include, for example, a backing, active compound reservoir, a control membrane, liner and contact adhesive. Such transdermal patches may be used to provide continuous pulsatile, or on demand delivery of the compounds of the present invention as desired.

The compositions of the invention can be formulated so as to provide quick, sustained or delayed release of the active ingredient after administration to the patient by employing procedures known in the art. Controlled release drug delivery systems include osmotic pump systems and dissolutional systems containing polymer-coated reservoirs or drug-polymer matrix formulations. Examples of controlled release systems are given in U.S. Pat. Nos. 3,845,770 and 4,326,525 and in P. J. Kuzma et al, Regional Anesthesia 22 (6): 543-551 (1997), all of which are incorporated herein by reference.

The compositions of the invention can also be delivered through intra-nasal drug delivery systems for local, systemic, and nose-to-brain medical therapies. Controlled Particle Dispersion (CPD)™ technology, traditional nasal spray bottles, inhalers or nebulizers are known by those skilled in the art to provide effective local and systemic delivery of drugs by targeting the olfactory region and paranasal sinuses.

The invention also relates to an intravaginal shell or core drug delivery device suitable for administration to the human or animal female. The device may be comprised of the active pharmaceutical ingredient in a polymer matrix, surrounded by a sheath, and capable of releasing the compound in a substantially zero order pattern on a daily basis similar to devises used to apply testosterone as described in PCT Patent No. WO 98/50016.

Current methods for ocular delivery include topical administration (eye drops), subconjunctival injections, periocular injections, intravitreal injections, surgical implants and iontophoresis (uses a small electrical current to transportionized drugs into and through body tissues). Those skilled in the art would combine the best suited excipients with the compound for safe and effective intra-occular administration.

The most suitable route will depend on the nature and severity of the condition being treated. Those skilled in the art are also familiar with determining administration methods (oral, intravenous, inhalation, sub-cutaneous, rectal etc.), dosage forms, suitable pharmaceutical excipients and other matters relevant to the delivery of the compounds to a subject in need thereof.

### Combination Therapy

The compounds of the invention may be usefully combined with one or more other compounds of the invention or one or more other therapeutic agent or as any combination thereof, in the treatment of iron disorders. For example, a compound of the invention may be administered simultaneously, sequentially or separately in combination with other therapeutic agents, including, but not limited to iron chelators; e.g. deferasirox (ICL-670), deferiprone and desferroxamine; and erythropoietin (EPO), e.g. rh-EPO. In addition, compounds of the invention, as inhibitors of DMT1 activity, could also be combined with phlebotomy therapy for the treatment of iron overload disorders.

As used herein “combination” refers to any mixture or permutation of one or more compounds of the invention and one or more other compounds of the invention or one or more additional therapeutic agent. Unless the context makes clear otherwise, “combination” may include simultaneous or sequentially delivery of a compound of the invention with one or more therapeutic agents. Unless the context makes clear otherwise, “combination” may include dosage forms of a compound of the invention with another therapeutic agent. Unless the context makes clear otherwise, “combination” may include routes of administration of a compound of the invention with another therapeutic agent. Unless the context makes clear otherwise, “combination” may include formulations of a compound of the invention with another therapeutic agent. Dosage forms, routes of administration and pharmaceutical compositions include, but are not limited to, those described herein.

### Kits-Of-Parts

The present invention also provides kits that contain a pharmaceutical composition which includes one or more compounds of the invention. The kit also includes instructions for the use of the pharmaceutical composition for treating iron disorders as well as other utilities as disclosed herein. Preferably, a commercial package will contain one or more unit doses of the pharmaceutical composition. For example, such a unit dose may be an amount sufficient for the preparation of an intravenous injection. It will be evident to those of ordinary skill in the art that compounds which are light and/or air sensitive may require special packaging and/or formulation. For example, packaging may be used which is opaque to light, and/or sealed from contact with ambient air, and/or formulated with suitable coatings or excipients.

### Preparation of the Compounds of the Invention

The following Reaction Schemes illustrate methods to make compounds of the invention, i.e., compounds of formula (I):

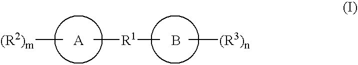

wherein

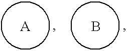

, m, n, R¹, R² and R³ are as defined above in the Summary of the Invention for compounds of formula (I), as a stereoisomer, enantiomer, tautomer thereof or mixtures thereof; or a pharmaceutically acceptable salt, solvate or prodrug thereof.

It is understood that in the following description, combinations of substituents and/or variables of the depicted formulae are permissible only if such contributions result in stable compounds.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters.

Protecting groups may be added or removed in accordance with standard techniques, which are known to one skilled in the art and as described herein.

The use of protecting groups is described in detail in Greene, T. W. and P. G. M. Wuts, Protective Groups in Organic Synthesis (2006), 4^{th} Ed., Wiley. The protecting group may also be a polymer resin such as a Wang resin or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of the invention which are pharmacologically active. Such derivatives may therefore be described as “prodrugs”. All prodrugs of compounds of this invention are included within the scope of the invention.

The following Reaction Schemes illustrate methods to make compounds of this invention. It is understood that one skilled in the art would be able to make these compounds by similar methods or by methods known to one skilled in the art. It is also understood that one skilled in the art would be able to make in a similar manner as described below other compounds of the invention not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Smith, M. B. and J. March, Advanced Organic Chemistry: Reactions, Mechanisms, and Structure, 5th edition (Wiley, December 2000)) or prepared as described herein.

The starting materials for the reaction schemes described below are commercially available or can be prepared according to methods known to one skilled in the art or by methods disclosed herein.

In the following Reaction Schemes, the R groups, e.g., R¹, R^{2a}, and R^{3a}, are defined as set forth above in the Summary of the Invention for compounds of formula (I), formula (Ia), formula (Ib), formula (Ic) and formula (Id) unless specifically defined otherwise.

### A. Preparation of Compounds of Formula (Ia)

Compounds of formula (Ia), as set forth above in the Embodiments of the Invention, are compounds of formula (I), as set forth above in the Summary of the Invention, and can be synthesized following the general procedure described below in Reaction Scheme 1 where R¹ is —C(O)—, R^{2a} and R^{3a} are both —R⁶—S—C(═NR⁴)N(R⁴)R⁵ (where each R⁴ is hydrogen, R⁵ is hydrogen and R⁶ is methylene) and R^{2b}, R^{2c}, R^{2d}, R^{2e}, R^{3b}, R^{3c}, R^{3d} and R^{3e} are each as described above in the Embodiments of the Invention.

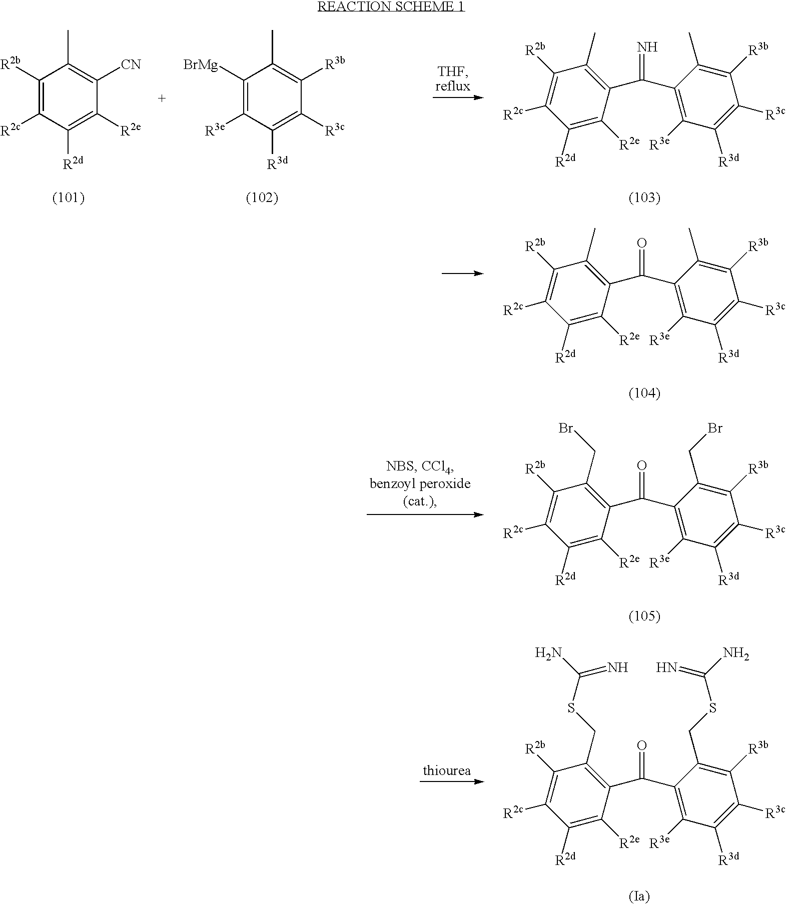

The starting materials for the above reaction scheme are commercially available or can be prepared according to methods known to one skilled in the art or by methods disclosed herein. In general, compounds of formula (Ia) are prepared in Reaction Scheme 1 as follows:

A cyano compound of formula (101) reacts with a Gringard reagent of formula (102) under reflux to afford the imine compound of formula (103), which is hydrolyzed to form the ketone compound of formula (104) under acidic conditions. Bromination of a compound of formula (104) with N-bromosuccinimide generates a di-bromo compound of formula (105) and subsequent displacement of the bromo groups with thiourea affords a compound of formula (Ia) of the invention.

Alternatively, the compounds of formula (Ia) of this invention can be synthesized following the general procedure as described below in Reaction Scheme 1A where R¹ is —C(O)—, R^{2a} and R^{3a} are both —R⁶—S—C(═NR⁴)N(R⁴)R⁵ (where each R⁴ is hydrogen, R⁵ is hydrogen and R⁶ is methylene) and R^{2b}, R^{2c}, R^{2d}, R^{2e}, R^{3b}, R^{3c}, R^{3d} and R^{3e} are each as described above in the Embodiments of the Invention, and X is chloro or bromo:

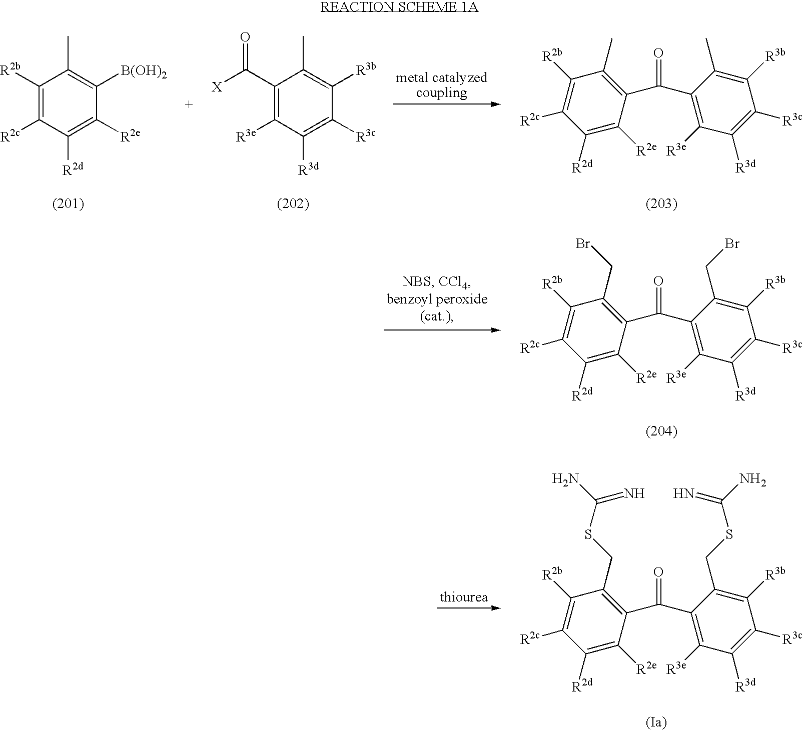

The starting materials for the above reaction scheme are commercially available or can be prepared according to methods known to one skilled in the art or by methods disclosed herein. In general, compounds of formula (Ia) are prepared in Reaction Scheme 1A as follows:

A boronic acid of formula (201) is coupled with an acid halide compound of formula (202) under metal catalyzed coupling reaction conditions in the presence of a metal catalyst, such as, but not limited, tetrakis(triphenylphosphene)palladium(0), and a base, such as, but not limited to, cesium carbonate, to afford a ketone compound of formula (203). Bromination of the compound of formula (203) with N-bromosuccinimide generates a di-bromo compound of formula (204) and subsequent displacement of the bromo groups with thiourea affords a compound of formula (Ia) of the invention.

Alternatively, the compounds of formula (Ia) of this invention can be synthesized following the general procedure as described below in Reaction Scheme 1B where R¹ is —O—, —S(O)ₚ— (where p is 0, 1 or 2) or —N(R⁴)—, R^{2a} and R^{3a} are both —R⁶—S—C(═NR⁴)N(R⁴)R⁵ (where each R⁴ is hydrogen, R⁵ is hydrogen and R⁶ is methylene) and R^{2b}, R^{2c}, R^{2d}, R^{2e}, R^{3b}, R^{3c}, R^{3d} and R^{3e} are each as described above in the Embodiments of the Invention, and X is chloro or bromo, and each R′ is independently alkyl or aralkyl:

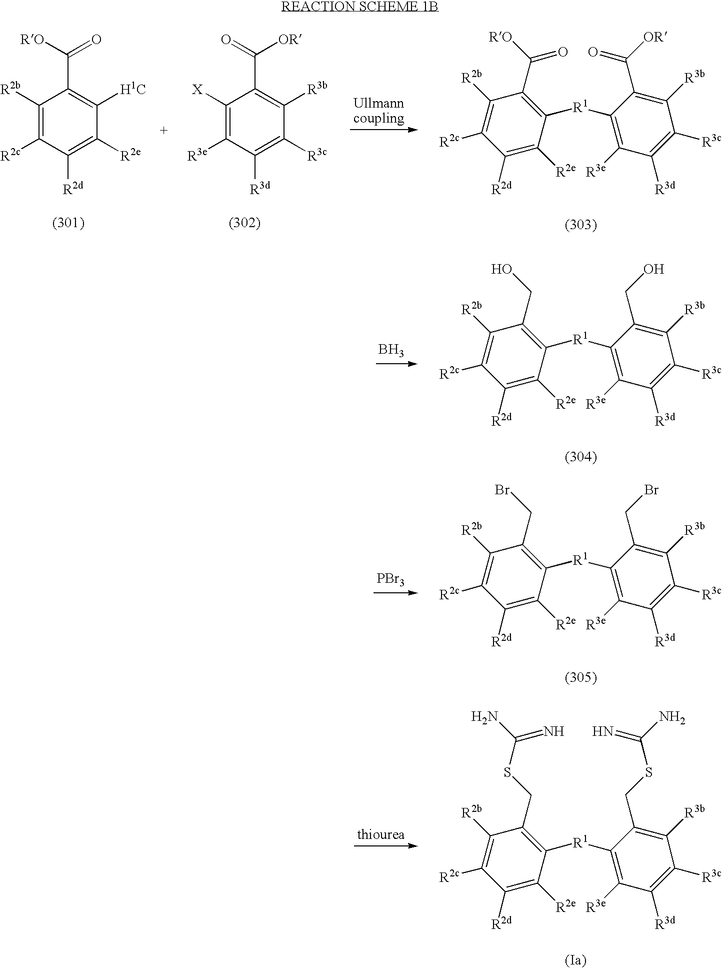

The starting materials for the above reaction scheme are commercially available or can be prepared according to methods known to one skilled in the art or by methods disclosed herein. In general, compounds of formula (Ia) are prepared in Reaction Scheme 1B as follows:

Compound of formula (301) is coupled with compound of formula (302) under Ullmann coupling conditions in the presence of copper powder at 120-200° C. to afford the di-acid compound of formula (303). Reduction of the di-acid with a reducing agent, such as, but not limited to, borane-tetrahydrofuran complex generates di-hydroxyl compound (304). Reaction of the di-hydroxyl compound of formula (304) with phosphorus tribromide affords the di-bromo compound of formula (305), and subsequent displacement of bromo groups with thiourea affords the compound of formula (Ia) of the invention.

Alternatively, the compounds of formula (Ia) of this invention can be synthesized following the general procedure as described below in Reaction Scheme 1C where R¹ is —O—, —S(O)ₚ— (where p is 0, 1 or 2) or —N(R⁴)—, R^{2a} and R^{3a} are both —R⁶—S—C(═NR⁴)N(R⁴)R⁵ (where each R⁴ is hydrogen, R⁵ is hydrogen and R⁶ is methylene) and R^{2b}, R^{2c}, R^{2d}, R^{2e}, R^{3b}, R^{3c}, R^{3d} and R^{3e} are each as described above in the Embodiments of the Invention, and X is chloro or bromo:

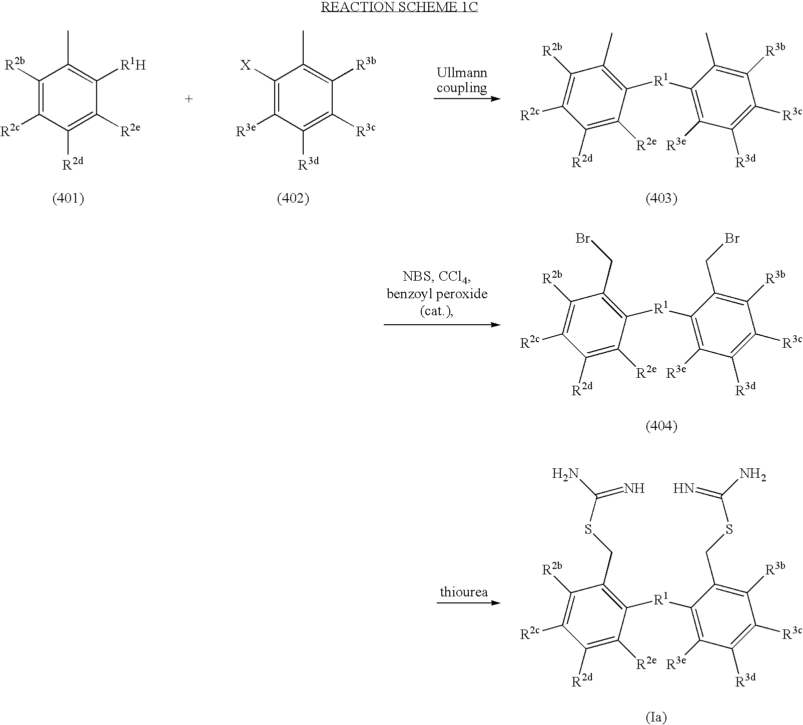

The starting materials for the above reaction scheme are commercially available or can be prepared according to methods known to one skilled in the art or by methods disclosed herein. In general, compounds of formula (Ia) are prepared in Reaction Scheme 1C as follows:

Compound of formula (401) is coupled with compound of formula (402) under Ullmann coupling conditions in the presence of Cu at 120-200° C. to afford the di-aryl compound of formula (403). Bromination of the compound of formula (403) with N-bromosuccinimide affords the di-bromo compound of formula (404) and subsequent displacement of the bromo groups with thiourea affords the compound of formula (Ia) of the invention.

Alternatively, the compounds of formula (Ia) of this invention can be synthesized following the general procedure as described below in Reaction Scheme 1D where R¹ is —O—, —S(O)ₚ— (where p is 0, 1 or 2) or —N(R⁴)—, R^{2a} and R^{3a} are both —R⁶—C(═NCN)N(R⁴)R⁵ (where R⁴ is hydrogen, R⁵ is hydrogen and R⁶ is ethylene) or R^{2a} and R^{3a} are both —R⁶—C(═NR⁴)N(R⁴)R⁵ (where each R⁴ is hydrogen, R⁵ is hydrogen and R⁶ is ethylene), and R^{2b}, R^{2c}, R^{2d}, R^{2e}, R^{3b}, R^{3c}, R^{3d} and R^{3e} are each as described above in the Embodiments of the Invention, and X is chloro or bromo, and R′ is alkyl:

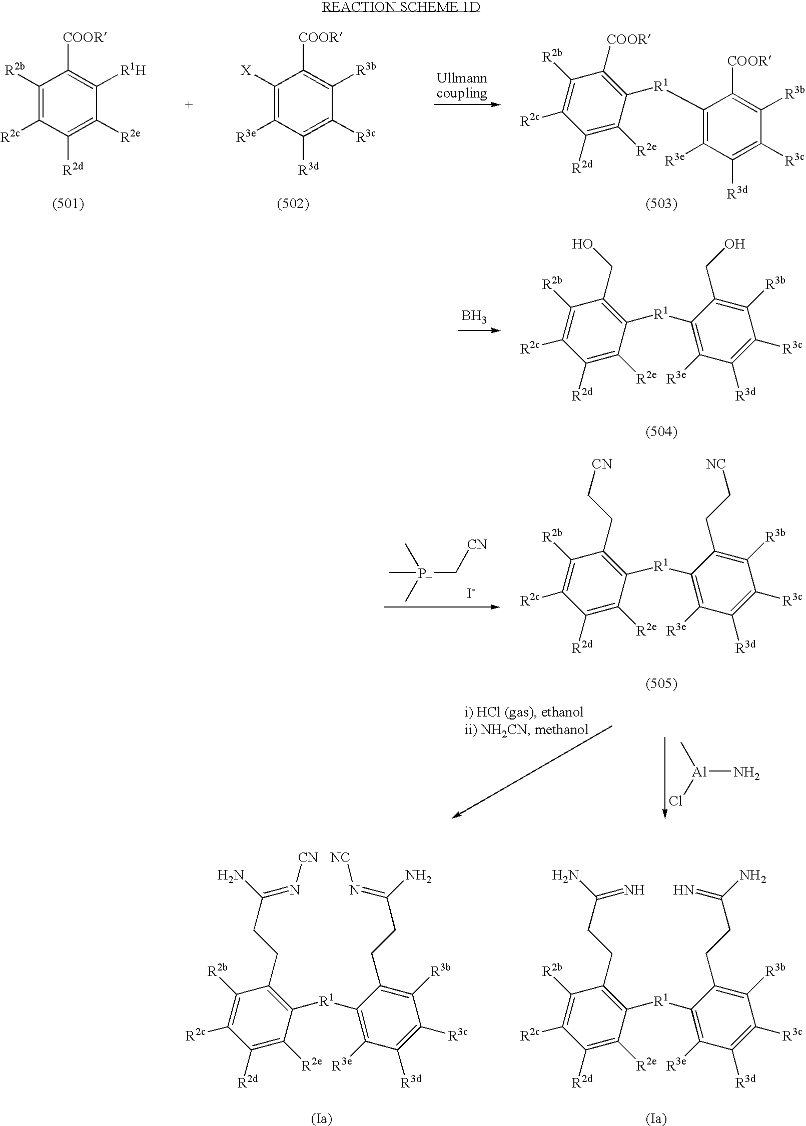

The starting materials for the above reaction scheme are commercially available or can be prepared according to methods known to one skilled in the art or by methods disclosed herein. In general, compounds of formula (Ia) are prepared in Reaction Scheme 1D as follows:

A compound of formula (501) is coupled with a compound of formula (502) under Ullmann coupling conditions in the presence of copper at 120-200° C. to afford a compound of formula (503). Reduction of the carboxylate groups of a compound of formula (503) with a reducing agent, such as, but not limited to, borane-tetrahydrofuran complex, generates the di-hydroxyl compound of formula (504). The compound of formula (504) reacts with (cyanomethyl)trimethylphosphonium iodide in the presence of a base, such as, but not limited to, diisopropylethylamine, to generate the cyano compound of formula (505). The compound of formula (505) reacts with amino(methyl)aluminum chloride to afford the compound of formula (Ia) of the invention where R^{2a} and R^{3a} are both —R⁶—C(═NR⁴)N(R⁴)R⁵ (where each R⁴ is hydrogen, R⁵ is hydrogen and R⁶ is ethylene). Alternatively, a compound of formula (505) is treated with hydrogen chloride gas in an alcohol solvent, such as, but not limited to, ethanol, followed by the reaction with cyanamide to afford the compound of formula (Ia) of the invention where R^{2a} and R^{3a} are both —R⁶—C(═NCN)N(R⁴)R⁵ (where R⁴ is hydrogen, R⁵ is hydrogen and R⁶ is ethylene).

Alternatively, the compounds of formula (Ia) of this invention can be synthesized following the general procedure described below in Reaction Scheme 1E where R¹ is —O—, —S(O)ₚ— (where p is 0, 1 or 2) or —N(R⁴)—, R^{2a} and R^{3a} are both —R⁶—C(═NR⁴)N(R⁴)R⁵ (where each R⁴ is hydrogen, R⁵ is hydrogen and R⁶ is propylene), and R^{2b}, R^{2c}, R^{2d}, R^{2e}, R^{3b}, R^{3c}, R^{3d} and R^{3e} are each as described above in the Embodiments of the Invention, and X is chloro or bromo:

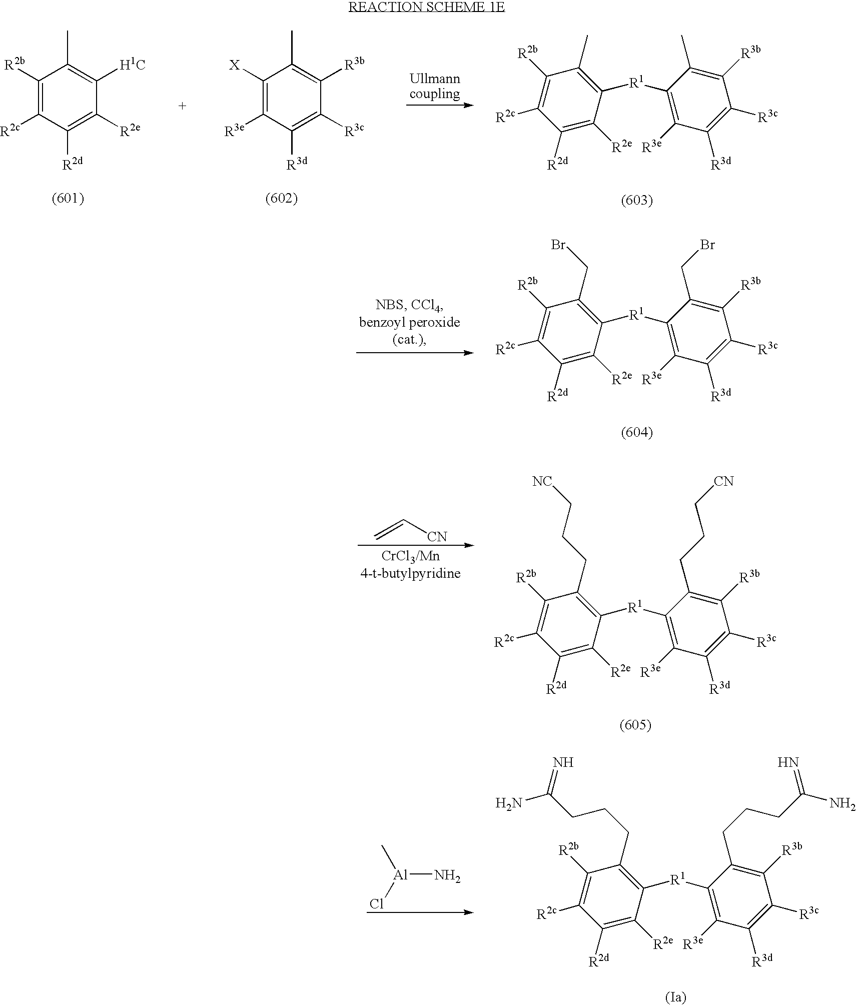

The starting materials for the above reaction scheme are commercially available or can be prepared according to methods known to one skilled in the art or by methods disclosed herein. In general, compounds of formula (Ia) are prepared in Reaction Scheme 1E as follows:

A compound of formula (601) is coupled with a compound of formula (602) under Ullmann coupling conditions in the presence of copper powder at 120-200° C. to afford the di-aryl compound of formula (603). Bromination of compound (603) with N-bromosuccinimide affords the di-bromo compound of formula (604) which is subsequently coupled with acrylonitrile via chromium(III) mediated coupling reaction to generate a compound of formula (605). A compound of formula (505) reacts with amino(methyl)aluminum chloride to afford a compound of formula (Ia) of the invention.

Alternatively, the compounds of formula (Ia) of this invention can be synthesized following the general procedure as described below in Reaction Scheme 1F where R¹ is —O—, —S(O)ₚ— (where p is 0, 1 or 2) or —N(R⁴)—, R^{2a} and R^{3a} are both —R⁶—N(R⁷)C(═NR⁴)N(R⁴)R⁵ (where each R⁴ is hydrogen, R⁵ is hydrogen, R⁶ is ethylene and R⁷ is hydrogen), and R^{2b}, R^{2c}, R^{2d}, R^{2e}, R^{3b}, R^{3c}, R^{3d} and R^{3e} are each as described above in the Embodiments of the Invention, and X is chloro or bromo:

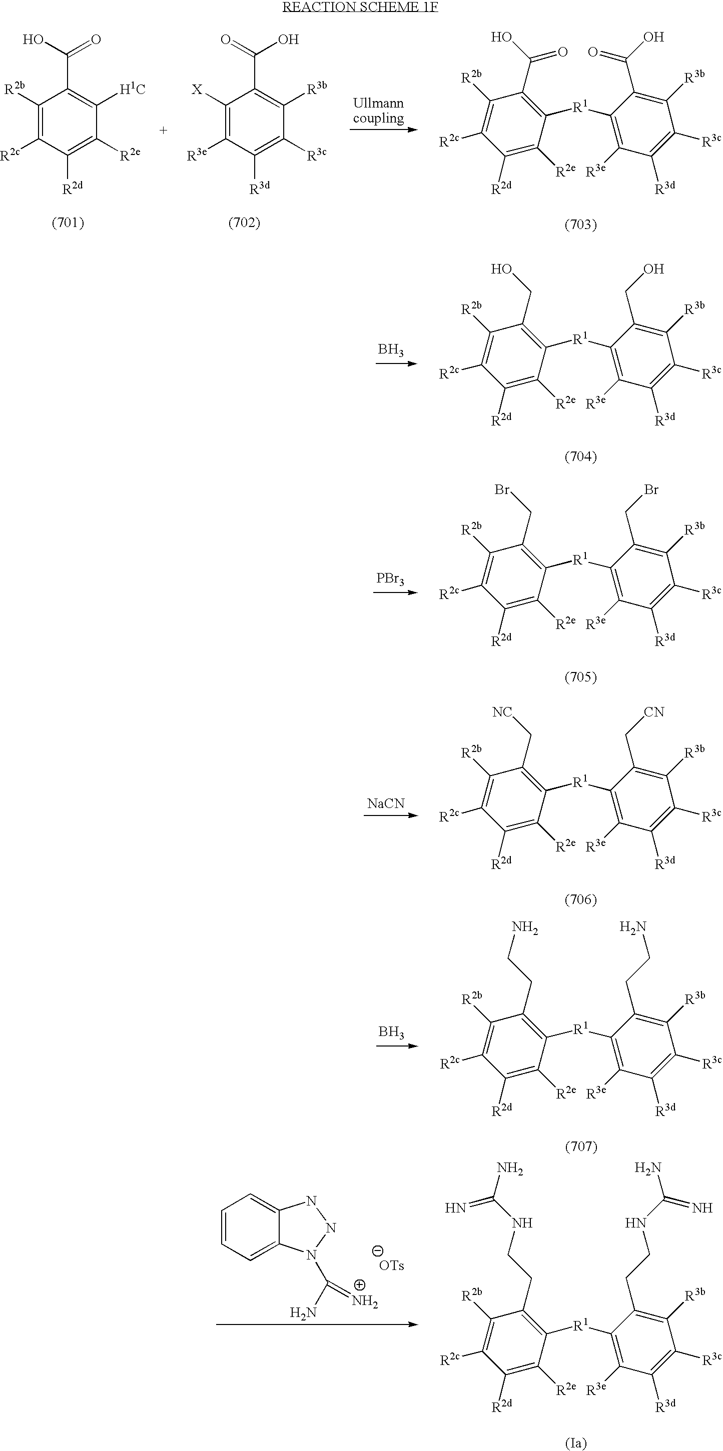

The starting materials for the above reaction scheme are commercially available or can be prepared according to methods known to one skilled in the art or by methods disclosed herein. In general, compounds of formula (Ia) are prepared in Reaction Scheme 1F as follows:

A compound of formula (701) is coupled with a compound of formula (702) under Ullmann coupling conditions in the presence of copper at 120-200° C. to afford the di-acid compound of formula (703). Reduction of the carboxylate groups of compound of formula (703) with a reducing agent, such as, but not limited to, borane-tetrahydrofuran complex generates a di-hydroxyl compound of formula (704). Displacement of the hydroxyl groups of the compound of formula (704) with bromo groups generates the di-bromo compound of formula (705). Subsequent displacement of the bromo groups with cyano groups generates a compound of formula (706) which is reduced by a reducing agent, such as, but not limited to, boran-tetrahydrofuran complex to afford the diamino compound of formula (707). Reaction between a compound of formula (707) and an amidinium reagent, such as, but not limited to, 1-benzotriazolecarboxamidinium tosylate, in the presence of a base, such as, but not limited to, diisopropylethylamine, affords a compound of formula (Ia) of the invention.

### B. Preparation of Compounds of Formula (Ib)

Compounds of formula (Ib), as set forth above in the Embodiments of the Invention, are compounds of formula (I), as set forth above in the Summary of the Invention, and can be synthesized following the general procedure as described below in Reaction Scheme 2 where R¹ is —C(O)—, R^{2a} and R^{3a} are both —R⁶—S—C(═NR⁴)N(R⁴)R⁵ (where each R⁴ is hydrogen, R⁵ is hydrogen and R⁶ is methylene), and R^{2b}, R^{2c}, R^{2d} R^{2e}, R^{2f}, R^{2g}, R^{3b}, R^{3c}, R^{3d} and R^{3e} are each as described above in the Embodiments of the Invention.

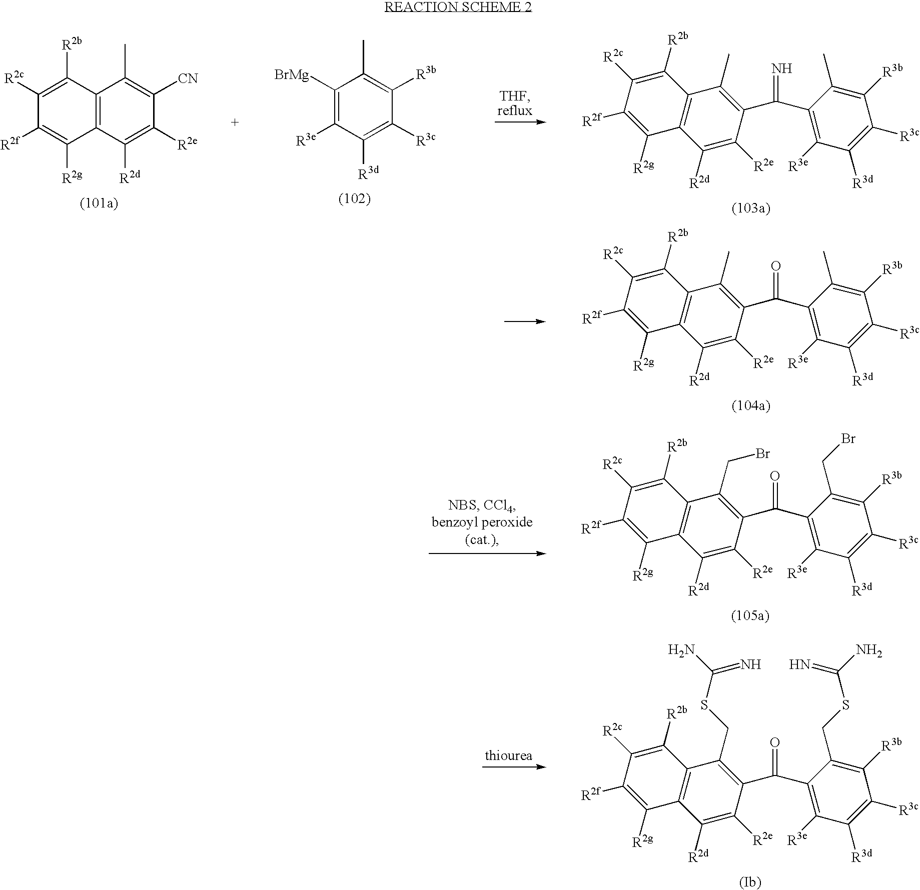

The starting materials for the above reaction scheme are commercially available or can be prepared according to methods known to one skilled in the art or by methods disclosed herein. In general, compounds of formula (Ib) are prepared in Reaction Scheme 2 as follows:

A cyano compound of formula (101a) reacts with a Gringard reagent of formula (102) under reflux to afford the imine compound of formula (103a), which is hydrolyzed to form the ketone compound of formula (104a) under acidic conditions. Bromination of compound (104a) with N-bromosuccinimide generates a di-bromo compound of formula (105a) and subsequent displacement of the bromo groups with thiourea affords a compound of formula (Ib) of the invention.

Alternatively, the compounds of formula (Ib) of this invention can be synthesized following the general procedure as described below in Reaction Scheme 2A where R¹ is —C(O)—, R^{2a} and R^{3a} are both —R⁶—S—C(═NR⁴)N(R⁴)R⁵ (where each R⁴ is hydrogen, R⁵ is hydrogen and R⁶ is methylene) and R^{2b}, R^{2c}, R^{2d}, R^{2e}, R^{2f}, R^{2g}, R^{3b}, R^{3c}, R^{3d} and R^{3e} are each as described above in the Embodiments of the Invention, and X is chloro or bromo:

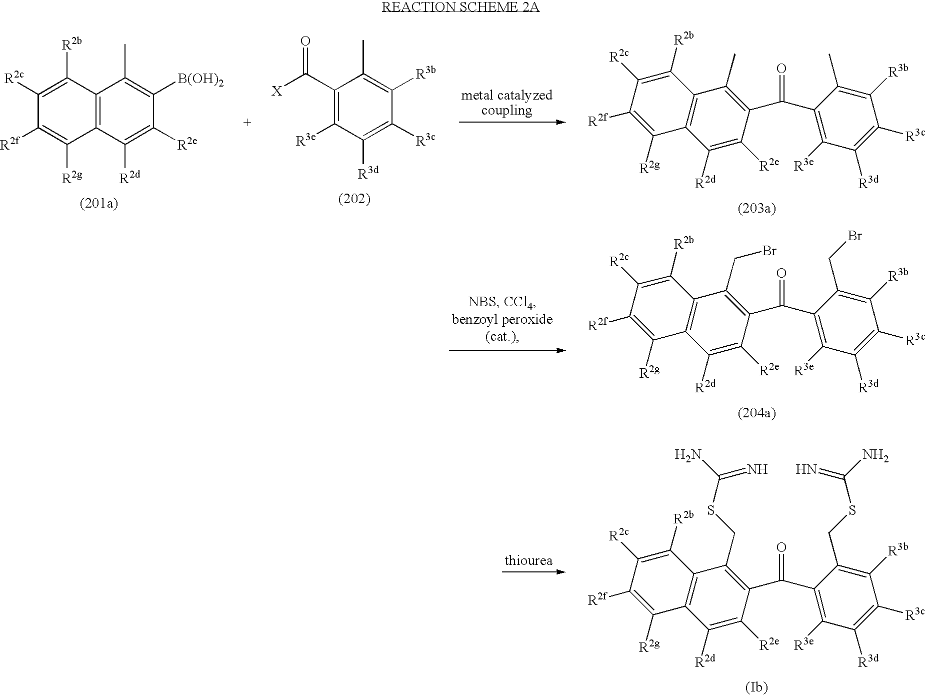

The starting materials for the above reaction scheme are commercially available or can be prepared according to methods known to one skilled in the art or by methods disclosed herein. In general, compounds of formula (Ib) are prepared in Reaction Scheme 2A as follows:

A boronic acid of formula (201a) is coupled with an acid halide compound of formula (202) under metal catalyzed coupling reaction conditions in the presence of a metal catalyst, such as, but not limited to, tetrakis(triphenylphosphene)palladium(0), and a base, such as, but not limited to, cesium carbonate, to afford the ketone compound of formula (203a). Bromination of the compound of formula (203a) with N-bromosuccinimide generates the di-bromo compound of formula (204a) and subsequent displacement of the bromo groups with thiourea affords a compound of formula (Ib) of the invention.

Alternatively, the compounds of formula (Ib) of this invention can be synthesized following the general procedure as described below in Reaction Scheme 2B where R¹ is —O—, —S(O)ₚ— (where p is 0, 1 or 2) or —N(R⁴)—, R^{2a} and R^{3a} are both —R⁶—S—C(═NR⁴)N(R⁴)R⁵ (where each R⁴ is hydrogen, R⁵ is hydrogen and R⁶ is methylene) and R^{2b}, R^{2c}, R^{2d}, R^{2e}, R^{2f}, R^{2g}, R^{3b}, R^{3c}, R^{3d}, and R^{3e} are each as described above in the Embodiments of the Invention, and X is chloro or bromo, and each R′ is independently alkyl or aralkyl:

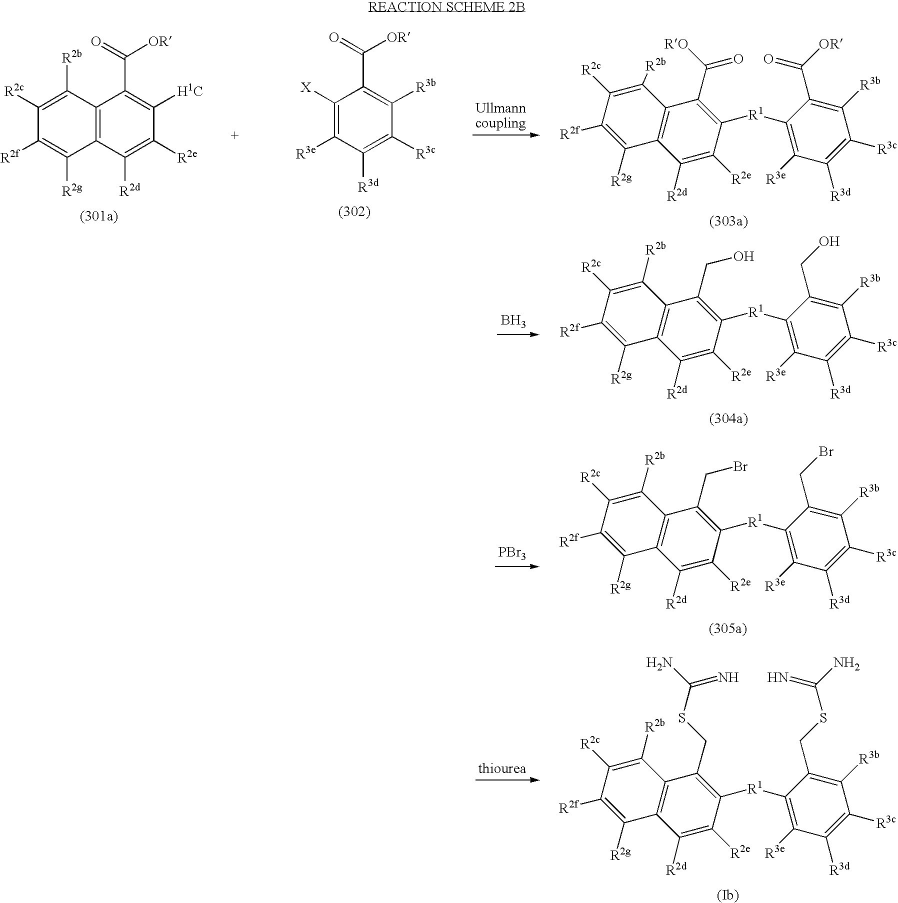

The starting materials for the above reaction scheme are commercially available or can be prepared according to methods known to one skilled in the art or by methods disclosed herein. In general, compounds of formula (Ib) are prepared in Reaction Scheme 2B as follows:

Compound of formula (301a) is coupled with compound of formula (302) under Ullmann coupling conditions in the presence of copper powder at 120-200° C. to afford the di-acid compound of formula (303a). Reduction of the di-acid with a reducing agent, such as, but not limited to, borane-tetrahydrofuran complex generates di-hydroxyl compound (304a). Reaction of the di-hydroxyl compound of formula (304a) with phosphorus tribromide affords the di-bromo compound of formula (305a), and subsequent displacement of bromo groups with thiourea affords a compound of formula (Ib) of the invention.

Alternatively, the compounds of formula (Ib) of this invention can be synthesized following the general procedure as described below in Reaction Scheme 2C where R¹ is —O—, —S(O)ₚ— (where p is 0, 1 or 2) or —N(R⁴)—, R^{2a} and R^{3a} are both —R⁶—S—C(═NR⁴)N(R⁴)R⁵ (where each R⁴ is hydrogen, R⁵ is hydrogen and R⁶ is methylene) and R^{2b}, R^{2c}, R^{2d}, R^{2e}, R^{2f}, R^{2g}, R^{3b}, R^{3c}, R^{3d} and R^{3e} are each as described above in the Embodiments of the Invention, and X is chloro or bromo:

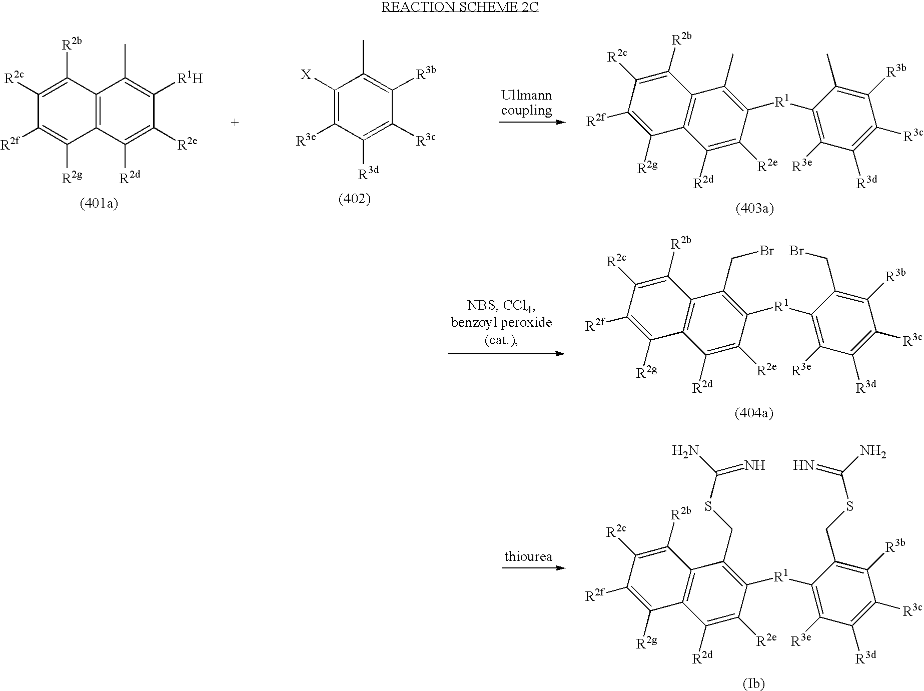

The starting materials for the above reaction scheme are commercially available or can be prepared according to methods known to one skilled in the art or by methods disclosed herein. In general, compounds of formula (Ib) are prepared in Reaction Scheme 2C as follows:

Compound of formula (401a) is coupled with compound of formula (402) under Ullmann coupling conditions in the presence of copper powder at 120-200° C. to afford the di-aryl compound of formula (403a). Bromination of the compound of formula (403a) with N-bromosuccinimide affords the di-bromo compound of formula (404a) and subsequent displacement of the bromo groups with thiourea affords a compound of formula (Ib) of the invention.

### C. Preparation of Compounds of Formula (Ic)

Compounds of formula (Ic), as set forth above in the Embodiments of the Invention, are compounds of formula (I), as set forth above in the Summary of the Invention, and can be synthesized following the general procedure described below in Reaction Scheme 3 where R¹ is —O—, —S(O)ₚ— (where p is 0, 1 or 2) or —N(R⁴)—, R^{2a} and R^{3a} are both —R⁶—S—C(═NR⁴)N(R⁴)R⁵ (where each R⁴ is hydrogen, R⁵ is hydrogen and R⁶ is methylene), and Y, R^{2b}, R^{2c}, R^{3b}, R^{3c}, R^{3d} and R^{3e} are each as described in the Embodiment of the Invention, and X is chloro or bromo, and each R′ is independently alkyl or aralkyl:

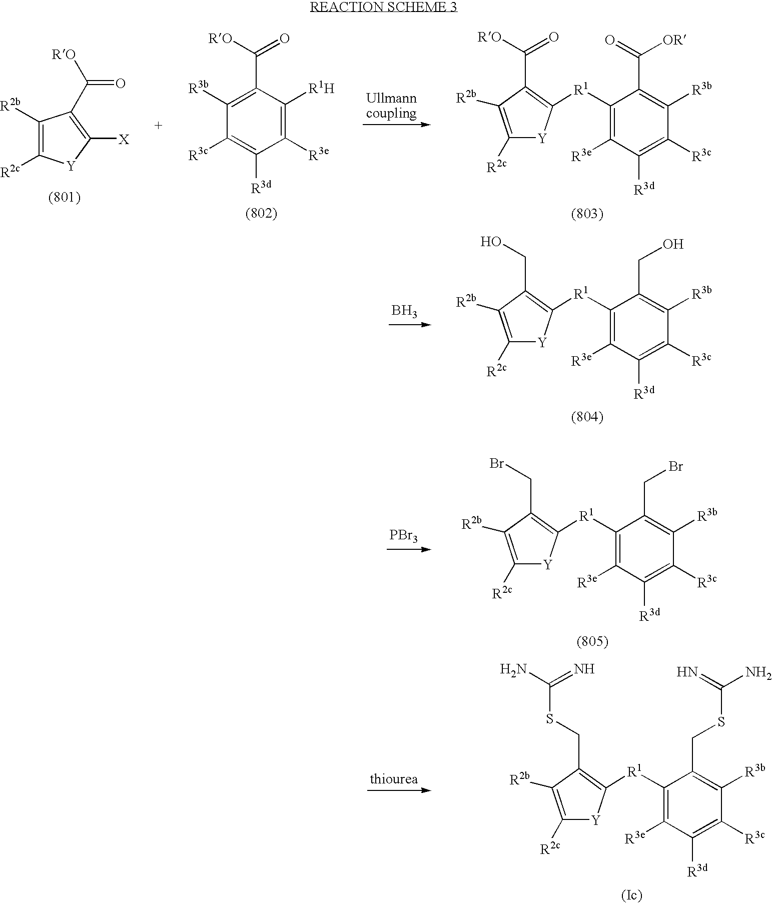

The starting materials for the above reaction scheme are commercially available or can be prepared according to methods known to one skilled in the art or by methods disclosed herein. In general, compounds of formula (Ic) are prepared in Reaction Scheme 3 as follows:

A compound of formula (801) is coupled with compound of formula (802) under Ullmann coupling conditions in the presence of copper powder at 120-200° C. to afford the di-acid compound of formula (803). Reduction of the di-acid compound with a reducing agent, such as, but not limited to, borane-tetrahydrofuran complex generates a di-hydroxyl compound of formula (804). Displacement of the hydroxyl groups with bromo groups of the di-dihydroxyl compound of formula (804) with phosphorus tribromide affords a di-bromo compound of formula (805), and subsequent displacement of bromo groups with thiourea affords the compound of formula (Ic) of the invention.

### D. Preparation of Compounds of Formula (Id)

Compounds of formula (Id), as set forth above in the Embodiments of the Invention, are compounds of formula (I), as set forth above in the Summary of the Invention, and can be synthesized following the general procedure described below in Reaction Scheme 4 where R¹ is —O—, —S(O)ₚ— (where p is 0, 1 or 2) or —N(R⁴)—, R^{2a} and R^{3a} are both —R⁶—S—C(═NR⁴)N(R⁴)R⁵ (where each R⁴ is hydrogen, R⁵ is hydrogen and R⁶ is methylene), and Y, R^{2b}, R^{2c}, R^{3b}, R^{3c}, R^{3d} and R^{3e} are each as described in the Embodiment of the Invention, and X is chloro or bromo, and each R′ is independently alkyl or aralkyl:

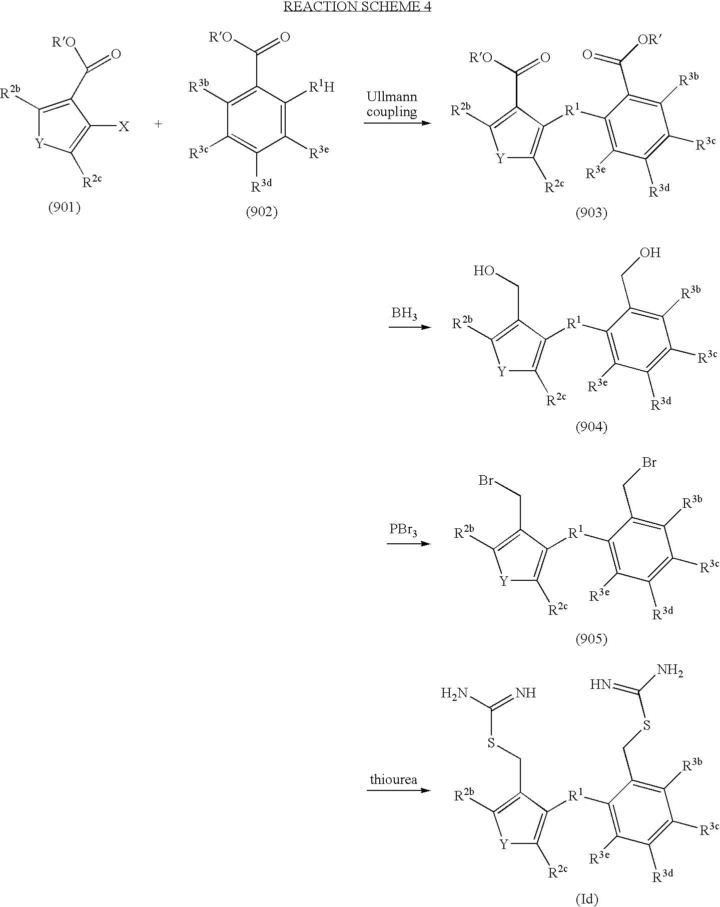

The starting materials for the above reaction scheme are commercially available or can be prepared according to methods known to one skilled in the art or by methods disclosed herein. In general, compounds of formula (Id) are prepared in Reaction Scheme 4 as follows:

A compound of formula (901) is coupled with compound of formula (902) under Ullmann coupling conditions in the presence of copper powder at 120-200° C. to afford the di-acid compound of formula (903). Reduction of the di-acid compound with a reducing agent, such as, but not limited to, borane-tetrahydrofuran complex generates a di-hydroxyl compound of formula (904). Displacement of the hydroxyl groups with bromo groups of the di-hydroxyl compound of formula (904) with phosphorus tribromide affords a di-bromo compound of formula (905), and subsequent displacement of bromo groups with thiourea affords the compound of formula (Id) of the invention.

All compounds of the invention as prepared above and below which exist in free base or acid form may be converted to their pharmaceutically acceptable salt by treatment with the appropriate inorganic or organic base or acid by methods known to one skilled in the art. Salts of the compounds prepared herein may be converted to their free base or acid by standard techniques known to one skilled in the art.

The following Preparations, which are directed to the preparation of intermediates used in the preparation of the compounds of formula (I), and the following Examples, which are directed to the preparation of the compounds of formula (1), are provided as a guide to assist in the practice of the invention, and are not intended as a limitation on the scope of the invention.

### Preparation 1

### Preparation of 1-(1-hydroxyethyl)-2-(6-(1-hydroxyethyl)phenoxy)benzene

To a stirred mixture of bis(2-formylphenyl)ether (2.26 g, 10.00 mmol) in dry ether was added the solution of methylmagnesium bromide (8.40 mL of 3 M solution, 25.0 mmol) dropwise. The mixture was slowly heated to reflux and stirred at reflux for 1 h, cooled to ambient temperature, washed with ammonium chloride solution and water, dried over sodium sulfate and filtered. The solvent was evaporated to afford 1-(1-hydroxyethyl)-2-(6-(1-hydroxyethyl)phenoxy)benzene in 84% yield (2.17 g): ¹H NMR (300 MHz, CDCl₃) δ 7.58-7.52 (m, 2H), 7.27-7.12 (m, 4H), 6.83-6.77 (m, 2H), 5.21 (q, J=6.5 Hz, 2H), 2.45-2.32 (m, 2H), 1.56 (d, J=6.5 Hz, 6H).

### Preparation 2

### Preparation of 2,2′-thiodibenzoic acid

A mixture of 2-bromobenzoic acid (4.02 g, 20.00 mmol), 2-mercaptobenzoic acid (3.08 g, 20.00 mmol), potassium carbonate (5.85 g, 42.00 mmol) and copper powder (1.27 g, 20.00 mmol) in water (20 mL) was heated in a sealed tube at 130-140° C. for 3 h, cooled, and filtered. The filtrate was acidified with concentrated hydrochloric acid to afford 2,2′-thiodibenzoic acid as a white solid in 98% yield (5.40 g): mp 226-227° C.; ¹H NMR (300 MHz, DMSO-d₆) δ 13.10 (s, 2H), 7.80 (dd, J=7.6, 1.5 Hz, 2H), 7.46-7.38 (m, 2H), 7.36-7.28 (m, 2H), 7.07 (dd, J=7.6, 1.2 Hz, 2H).

### Preparation 2.1

### Preparation of 6,6′-thiobis(3-fluorobenzoic acid)

Following the procedure as described in Preparation 2, making non-critical variations using 5-fluoro-2-mercaptobenzoic acid to replace 2-mercaptobenzoic acid to react with 2-bromo-5-fluorobenzoic acid, 6,6′-thiobis(3-fluorobenzoic acid) was obtained as a colorless solid in 92% yield: ¹H NMR (300 MHz, DMSO-d₆) δ 13.45 (s, 2H), 7.61 (dd, J=9.1, 2.9 Hz, 2H), 7.35-7.29 (m, 1H), 7.11 (dd, J=8.5, 5.3 Hz, 1H); MS (ES+) m/z (M−1) 309.0.

### Preparation 2.2

### Preparation of 2-(2-carboxyphenylthio)-5-fluorobenzoic acid

Following the procedure as described in Preparation 2, making non-critical variations using 5-fluoro-2-mercaptobenzoic acid to replace 2-mercaptobenzoic acid to react with 2-bromobenzoic acid, 2-(2-carboxyphenylthio)-5-fluorobenzoic acid was obtained as a colorless solid in 91% yield: ¹H NMR (300 MHz, DMSO-d₆) δ 13.28 (br s, 2H), 7.86-7.79 (m, 1H), 7.63-7.55 (m, 1H), 7.44-7.22 (m, 4H), 6.95-6.86 (m, 1H).

### Preparation 2.3

### Preparation of 2-(2-carboxyphenylthio)-4-methylbenzoic acid

Following the procedure as described in Preparation 2, making non-critical variations using 2-bromo-4-methylbenzoic acid to replace 2-bromobenzoic acid to react with 2-mercaptobenzoic acid, 2-(2-carboxyphenylthio)-4-methylbenzoic acid was obtained as a colorless solid in 96% yield: ¹H NMR (300 MHz, DMSO-d₆) δ 13.01 (s, 2H), 7.80 (dd, J=7.6, 1.5 Hz, 1H), 7.72 (d, J=7.9 Hz, 1H), 7.44-7.37 (m, 1H), 7.35-7.28 (m, 1H), 7.19-7.14 (m, 1H), 7.04 (dd, J=7.9, 1.1 Hz, 1H), 6.92 (s, 1H), 2.19 (s, 3H).

### Preparation 2.4

### Preparation of 2-(2-carboxyphenylthio)-5-methoxybenzoic acid

Following the procedure as described in Preparation 2, making non-critical variations using 2-bromo-5-methoxybenzoic acid to replace 2-bromobenzoic acid to react with 2-mercaptobenzoic acid, 2-(2-carboxyphenylthio)-5-methoxybenzoic acid was obtained as a colorless solid in 93% yield: ¹H NMR (300 MHz, DMSO-d₆) δ 13.11 (s, 2H), 7.86-7.81 (m, 1H), 7.36-7.28 (m, 2H), 7.27-7.24 (m, 1H), 7.20-7.13 (m, 1H), 7.13-7.07 (m, 1H), 6.72-6.99 (m, 1H), 3.80 (s, 3H).

### Preparation 2.5

### Preparation of 2-(2-carboxy-4-fluorophenylthio)-4-methylbenzoic acid

Following the procedure as described in Preparation 2, making non-critical variations using 5-fluoro-2-mercaptobenzoic acid to replace 2-mercaptobenzoic acid to react with 2-bromo-4-methylbenzoic acid, 2-(2-carboxy-4-fluorophenylthio)-4-methylbenzoic acid was obtained as a colorless solid in 92% yield: ¹H NMR (300 MHz, DMSO-d₆) δ 13.1 (s, 2H), 7.74 (d, J=7.9 Hz, 1H), 7.59 (dd, J=9.1, 2.9 Hz, 1H), 7.39-7.30 (m, 1H), 7.22 (dd, J=8.5, 5.4 Hz, 1H), 7.14-7.07 (m, 1H), 6.75 (s, 1H), 2.16 (s, 3H).

### Preparation 2.6

### Preparation of 2-(2-carboxy-4-fluorophenylthio)-5-methoxybenzoic acid

Following the procedure as described in Preparation 2, making non-critical variations using 5-fluoro-2-mercaptobenzoic acid to replace 2-mercaptobenzoic acid to react with 2-bromo-5-methoxybenzoic acid, 2-(2-carboxy-4-fluorophenylthio)-5-methoxybenzoic acid was obtained as a colorless solid in 89% yield: ¹H NMR (300 MHz, DMSO-d₆) δ 13.30 (s, 2H), 7.59 (dd, J=9.2, 2.9 Hz, 1H), 7.32-7.21 (m, 3H), 7.09 (dd, J=8.7, 2.9 Hz, 1H), 6.81 (dd, J=8.7, 5.3 Hz, 1H), 3.78 (s, 3H).

### Preparation 2.7

### Preparation of 2-(2-carboxy-4-fluorophenylthio)-4-chlorobenzoic acid

Following the procedure as described in Preparation 2, making non-critical variations using 5-fluoro-2-mercaptobenzoic acid to replace 2-mercaptobenzoic acid to react with 2-bromo-4-chlorobenzoic acid, 2-(2-carboxy-4-fluorophenylthio)-4-chlorobenzoic acid was obtained as a colorless solid in 95% yield: ¹H NMR (300 MHz, DMSO-d₆) δ 13.0 (s, 2H), 7.87 (d, J=8.4 Hz, 1H), 7.64 (dd, J=9.0, 2.7 Hz, 1H), 7.53-7.38 (m, 2H), 7.30 (dd, J=8.4, 2.0 Hz, 1H), 6.68 (d, J=2.0 Hz, 1H).

### Preparation 2.8

### Preparation of 2-(2-carboxyphenylthio)-3-methylbenzoic acid

Following the procedure as described in Preparation 2, making non-critical variations using 2-bromo-3-methylbenzoic acid to replace 2-bromobenzoic acid to react with 2-mercaptobenzoic acid, 2-(2-carboxyphenylthio)-3-methylbenzoic acid was obtained as a colorless solid in 92% yield: ¹H NMR (300 MHz, DMSO-d₆) δ 13.09 (s, 2H), 7.89 (dd, J=7.7, 1.5 Hz, 1H), 7.50-7.40 (m, 3H), 7.33-7.23 (m, 1H), 7.18-7.09 (m, 1H), 6.48-6.43 (m, 1H), 2.19 (s, 3H).

### Preparation 2.9

### Preparation of 2-(2-carboxyphenylthio)-5-methylbenzoic acid

Following the procedure as described in Preparation 2, making non-critical variations using 2-bromo-5-methylbenzoic acid to replace 2-bromobenzoic acid to react with 2-mercaptobenzoic acid, 2-(2-carboxyphenylthio)-5-methylbenzoic acid was obtained as a colorless solid in 90% yield: ¹H NMR (300 MHz, DMSO-d₆) δ 13.07 (s, 2H), 7.80 (dd, J=7.7, 1.5 Hz, 1H), 7.59 (d, J=1.5 Hz, 1H), 7.40-7.33 (m, 1H), 7.31-7.21 (m, 2H), 7.10 (d, J=8.0 Hz, 1H), 6.92-6.87 (m, 1H), 2.31 (s, 3H).

### Preparation 2.10

### Preparation of 2-(2-carboxyphenylthio)-4-chlorobenzoic acid

Following the procedure as described in Preparation 2, making non-critical variations using 2-bromo-4-chlorobenzoic acid to replace 2-bromobenzoic acid to react with 2-mercaptobenzoic acid, 2-(2-carboxyphenylthio)-4-chlorobenzoic acid was obtained as a colorless solid in 95% yield: ¹H NMR (300 MHz, DMSO-d₆) δ 13.28 (s, 2H), 7.86 (d, J=8.4 Hz, 1H), 7.81 (dd, J=7.5, 1.7 Hz, 1H), 7.56-7.44 (m, 2H), 7.37-7.29 (m, 2H), 6.81 (d, J=2.0 Hz, 1H).

### Preparation 2.11

### Preparation of 2-(2-carboxyphenylthio)-4-fluorobenzoic acid

Following the procedure as described in Preparation 2, making non-critical variations using 2-bromo-4-fluorobenzoic acid to replace 2-bromobenzoic acid to react with 2-mercaptobenzoic acid, 2-(2-carboxyphenylthio)-4-fluorobenzoic acid was obtained as a colorless solid in 91% yield: ¹H NMR (300 MHz, DMSO-d₆) δ 13.20 (s, 2H), 7.94 (dd, J=8.7, 6.2 Hz, 1H), 7.83-7.77 (m, 1H), 7.58-7.45 (m, 2H), 7.40-7.34 (m, 1H), 7.14-7.05 (m, 1H), 6.51 (dd, J=10.2, 2.6 Hz, 1H).

### Preparation 2.12

### Preparation of 2-(2-carboxyphenylthio)-1-naphthoic acid

Following the procedure as described in Preparation 2, making non-critical variations using 2-bromonaphthoic acid to replace 2-bromobenzoic acid to react with 2-mercaptobenzoic acid, 2-(2-carboxyphenylthio)-1-naphthoic acid was obtained as a colorless solid in 94% yield: ¹H NMR (300 MHz, DMSO-d₆) δ 13.20 (s, 2H), 8.20-8.12 (m, 2H), 8.06-8.03 (m, 1H), 7.93-7.90 (m, 1H), 7.69 (d, J=8.4 Hz, 1H), 7.61-7.52 (m, 2H), 7.16-7.06 (m, 2H), 6.29-6.26 (m, 1H).

### Preparation 2.13

### Preparation of 2-(2-carboxy-4-fluorophenylthio)-1-naphthoic acid

Following the procedure as described in Preparation 2, making non-critical variations using 5-fluoro-2-mercaptobenzoic acid to replace 2-mercaptobenzoic acid to react with 2-bromonaphthoic acid, 2-(2-carboxy-4-fluorophenylthio)-1-naphthoic acid was obtained as a colorless solid in 91% yield: ¹H NMR (300 MHz, DMSO-d₆) δ 13.65 (s, 1H), 13.41 (s, 1H), 8.21 (d, J=8.5 Hz, 1H), 8.20-8.15 (m, 2H), 7.74-7.68 (m, 2H), 7.66-7.57 (m, 2H), 7.18-7.05 (m, 1H), 6.32 (dd, J=5.2, 9.0 Hz, 1H).

### Preparation 2.14

### Preparation of 2-(2-carboxy-4-fluorophenylthio)-4-fluorobenzoic acid

Following the procedure as described in Preparation 2, making non-critical variations using 5-fluoro-2-mercaptobenzoic acid to replace 2-mercaptobenzoic acid to react with 2-bromo-4-fluorobenzoic acid, 2-(2-carboxy-4-fluorophenylthio)-4-fluorobenzoic acid was obtained as a colorless solid in 96% yield: ¹H NMR (300 MHz, DMSO-d₆) δ 13.33 (s, 2H), 7.95 (dd, J=8.7, 6.2 Hz, 1H), 7.63 (dd, J=9.0, 2.8 Hz, 1H), 7.53 (dd, J=8.7, 5.5 Hz, 1H), 7.46-7.38 (m, 1H), 7.11-7.01 (m, 1H), 6.40 (dd, J=10.3, 2.5 Hz, 1H).

### Preparation 2.15

### Preparation of 2-(2-carboxy-4-fluorophenylthio)-4,5-difluorobenzoic acid

Following the procedure as described in Preparation 2, making non-critical variations using 5-fluoro-2-mercaptobenzoic acid to replace 2-mercaptobenzoic acid to react with 2-bromo-4,5-difluorobenzoic acid, 2-(2-carboxy-4-fluorophenylthio)-4,5-difluorobenzoic acid was obtained as a colorless solid in 92% yield: ¹H NMR (300 MHz, DMSO-d₆) δ 13.52 (s, 2H), 7.89 (dd, J=10.9, 8.4 Hz, 1H), 7.63 (dd, J=9.0, 2.5 Hz, 1H), 7.45-7.32 (m, 2H), 6.85 (dd, J=11.4, 7.5 Hz, 1H).

### Preparation 2.16

### Preparation of 2-(2-carboxy-4-fluorophenylthio)-5-nitrobenzoic acid

Following the procedure as described in Preparation 2, making non-critical variations using 5-fluoro-2-mercaptobenzoic acid to replace 2-mercaptobenzoic acid to react with 2-bromo-5-nitrobenzoic acid, 2-(2-carboxy-4-fluorophenylthio)-5-nitrobenzoic acid was obtained as a yellow solid in 93% yield: ¹H NMR (300 MHz, DMSO-d₆) δ 13.65 (s, 2H), 8.60 (s, 1H), 8.14 (dd, J=8.9, 1.5 Hz, 1H), 7.78-7.61 (m, 2H), 7.57-7.45 (m, 1H), 6.83 (d, J=8.9 Hz, 1H).

### Preparation 2.17

### Preparation of 2-(2-carboxy-4-fluorophenylthio)-3-nitrobenzoic acid

Following the procedure as described in Preparation 2, making non-critical variations using 5-fluoro-2-mercaptobenzoic acid to replace 2-mercaptobenzoic acid to react with 2-bromo-3-nitrobenzoic acid, 2-(2-carboxy-4-fluorophenylthio)-3-nitrobenzoic acid was obtained as a yellow solid in 73% yield: ¹H NMR (300 MHz, DMSO-d₆) δ 13.62 (br s, 2H), 8.09 (dd, J=7.8, 1.4 Hz, 1H), 7.95 (dd, J=7.8, 1.4 Hz, 1H), 7.85-7.76 (m, 1H), 7.63 (dd, J=9.0, 3.0 Hz, 1H), 7.33-7.24 (m, 1H), 6.66 (dd, J=9.0, 5.1 Hz, 1H).

### Preparation 2.18

### Preparation of 2-(2-carboxy-4-(trifluoromethyl)phenylthio)-5-fluorobenzoic acid

Following the procedure as described in Preparation 2, making non-critical variations using 5-fluoro-2-mercaptobenzoic acid to replace 2-mercaptobenzoic acid to react with 2-chloro-5-(trifluoromethyl)benzoic acid, 2-(2-carboxy-4-(trifluoromethyl)phenylthio)-5-fluorobenzoic acid was obtained as a colorless solid in 95% yield: ¹H NMR (300 MHz, DMSO-d₆) δ 13.51 (brs, 2H), 8.10 (d, J=1.7 Hz, 1H), 7.73-7.56 (m, 3H), 7.51-7.41 (m, 1H), 6.87 (d, J=8.5 Hz, 1H).

### Preparation 2.19

### Preparation of 5-acetamido-2-(2-carboxyphenylthio)benzoic acid

Following the procedure as described in Preparation 2, making non-critical variations using 5-acetamido-2-bromobenzoic acid to replace 2-bromobenzoic acid to react with 2-mercaptobenzoic acid, 5-acetamido-2-(2-carboxyphenylthio)benzoic acid was obtained as a colorless solid in 98% yield: ¹H NMR (300 MHz, DMSO-d₆) δ 13.66 (br s, 2H), 10.22 (s, 1H), 8.04 (d, J=2.4 Hz, 1H), 7.81 (dd, J=7.7, 1.5 Hz, 1H), 7.63 (dd, J=8.6, 2.4 Hz, 1H), 7.38-7.30 (m, 1H), 7.26-7.17 (m, 2H), 6.84-6.77 (m, 1H), 2.03 (s, 3H).

### Preparation 2.20

### Preparation of 2-(2-carboxy-4-chlorophenylthio)-5-fluorobenzoic acid

Following the procedure as described in Preparation 2, making non-critical variations using 5-chloro-2-mercaptobenzoic acid to replace 2-mercaptobenzoic acid to react with 2-bromo-5-fluorobenzoic acid, 2-(2-carboxy-4-chlorophenylthio)-5-fluorobenzoic acid was obtained as a colorless solid in 92% yield: ¹H NMR (300 MHz, DMSO-d₆) δ 13.55 (s, 2H), 7.82 (d, J=2.5 Hz, 1H), 7.61 (dd, J=9.1, 2.5 Hz, 1H), 7.47 (dd, J=8.6, 2.5 Hz, 1H), 7.43-7.29 (m, 2H), 6.88 (d, J=8.6 Hz, 1H); MS (ES−) m/z 325.1 (M−1).

### Preparation 2.21

### Preparation of 6,6′-thiobis(3-chlorobenzoic acid)

Following the procedure as described in Preparation 2, making non-critical variations using 5-chloro-2-mercaptobenzoic acid to replace 2-mercaptobenzoic acid to react with 2-bromo-5-chlorobenzoic acid, 6,6′-thiobis(3-chlorobenzoic acid) was obtained as a colorless solid in 86% yield: ¹H NMR (300 MHz, DMSO-d₆) δ 13.50 (s, 2H), 7.82 (d, J=2.4 Hz, 2H), 7.52 (dd, J=8.5, 2.4 Hz, 2H), 7.11 (d, J=8.5 Hz, 2H).

### Preparation 2.22

### Preparation of 2-(2-(carboxymethyl)-4-fluorophenylthio)-5-fluorobenzoic acid

Following the procedure as described in Preparation 2, making non-critical variations using 5-fluoro-2-mercaptobenzoic acid to replace 2-mercaptobenzoic acid to react with 2-bromo-5-fluorophenylacetic acid, 2-(2-(carboxymethyl)-4-fluorophenylthio)-5-fluorobenzoic acid was obtained as a colorless solid in 98% yield: ¹H NMR (300 MHz, DMSO-d₆) δ 7.64 (dd, J=9.2, 3.0 Hz, 1H), 7.54 (dd, J=8.6, 6.0 Hz, 1H), 7.37 (dd, J=9.2, 3.0 Hz, 1H), 7.27-7.17 (m, 2H), 6.52 (dd, J=8.9, 5.1 Hz, 1H), 3.67 (s, 2H).

### Preparation 2.23

### Preparation of 2-(2-(carboxymethyl)-4-chlorophenylthio)-5-fluorobenzoic acid

Following the procedure as described in Preparation 2, making non-critical variations using 5-fluoro-2-mercaptobenzoic acid to replace 2-mercaptobenzoic acid to react with 2-bromo-5-chlorophenylacetic acid, 2-(2-(carboxymethyl)-4-chlorophenylthio)-5-fluorobenzoic acid was obtained as a colorless solid in 95% yield: MS (ES−) m/z 339.1 (M−1).

### Preparation 2.24

### Preparation of 2-(2-(carboxymethyl)phenylthio)benzoic acid

Following the procedure as described in Preparation 2, making non-critical variations using 2-bromophenylacetic acid to replace 2-bromobenzoic acid to react with 2-mercaptobenzoic acid, 2-(2-(carboxymethyl)phenylthio)benzoic acid was obtained as a colorless solid in 83% yield: ¹H NMR (300 MHz, DMSO-d₆) δ 12.78 (s, 2H), 7.88 (dd, J=7.7, 1.5 Hz, 1H), 7.51-7.40 (m, 3H), 7.39-7.31 (m, 1H), 7.31-7.24 (m, 1H), 7.19-7.11 (m, 1H), 6.50 (dd, J=8.2, 1.0 Hz, 1H), 3.64 (s, 2H).

### Preparation 2.25

### Preparation of 2-(2-carboxyphenylthio)-5-chlorobenzoic acid

Following the procedure as described in Preparation 2, making non-critical variations using 5-chloro-2-mercaptobenzoic acid to replace 2-mercaptobenzoic acid to react with 2-bromobenzoic acid, 2-(2-carboxyphenylthio)-5-chlorobenzoic acid was obtained as a colorless solid in 91% yield: ¹H NMR (300 MHz, DMSO-d₆) δ 13.29 (s, 2H), 7.82-7.79 (m, 2H), 7.47 (ddd, J=9.2, 8.1, 2.0 Hz, 2H), 7.41-7.34 (m, 1H), 7.14 (dd, J=7.5, 1.1 Hz, 1H), 7.04 (d, J=8.5 Hz, 1H).

### Preparation 2.26

### Preparation of 2-(2-carboxyphenylthio)-5-(trifluoromethyl)benzoic acid

Following the procedure as described in Preparation 2, making non-critical variations using 2-chloro-5-(trifluoromethyl)benzoic acid to replace 2-bromobenzoic acid to react with 2-mercaptobenzoic acid, 2-(2-carboxyphenylthio)-5-(trifluoromethyl)benzoic acid was obtained as a colorless solid in 88% yield: ¹H NMR (300 MHz, DMSO-d₆) δ 13.63 (s, 2H), 8.10 (d, J=1.6 Hz, 1H), 7.82 (dd, J=2.8, 6.3 Hz, 1H), 7.71 (dd, J=1.6, 8.6 Hz, 1H), 7.60-7.50 (m, 2H), 7.46 (dd, J=2.8, 6.3 Hz, 1H), 6.95 (d, J=8.6 Hz, 1H).

### Preparation 2.27

### Preparation of 2-(2-carboxy-4-(methylsulfonyl)phenylthio)-5-fluorobenzoic acid

Following the procedure as described in Preparation 2, making non-critical variations using 5-fluoro-2-mercaptobenzoic acid to replace 2-mercaptobenzoic acid to react with 2-chloro-5-(methylsulfonyl)benzoic acid, 2-(2-carboxy-4-(methylsulfonyl)phenylthio)-5-fluorobenzoic acid was obtained as a colorless solid in 53% yield: ¹H NMR (300 MHz, DMSO-d₆) δ 13.67 (br s, 2H), 8.31 (d, J=2.0 Hz, 1H), 7.82 (dd, J=8.5, 2.0 Hz, 1H), 7.67 (dd, J=9.1, 2.9 Hz, 1H), 7.63 (dd, J=8.5, 5.6 Hz, 1H), 7.53-7.42 (m, 1H), 6.87 (d, J=8.5 Hz, 1H), 3.19 (s, 3H).

### Preparation 2.28

### Preparation of 2-(2-carboxy-4-(N,N-dimethylsulfamoyl)phenylthio)-5-fluorobenzoic acid

Following the procedure as described in Preparation 2, making non-critical variations using 5-fluoro-2-mercaptobenzoic acid to replace 2-mercaptobenzoic acid to react with 2-chloro-5-dimethylsulfamoylbenzoic acid, 2-(2-carboxy-4-(N,N-dimethylsulfamoyl)phenylthio)-5-fluorobenzoic acid was obtained as a colorless solid in 89% yield: ¹H NMR (300 MHz, DMSO-d₆) δ 13.65 (s, 2H), 8.12 (d, J=2.1 Hz, 1H), 7.70-7.60 (m, 3H), 7.53-7.43 (m, 1H), 6.87 (d, J=8.5 Hz, 1H), 2.56 (s, 6H).

### Preparation 2.29

### Preparation of 4-(2-carboxy-4-fluorophenylthio)thiophene-3-carboxylic acid

Following the procedure as described in Preparation 2, making non-critical variations using 5-fluoro-2-mercaptobenzoic acid to replace 2-mercaptobenzoic acid to react with 4-bromothiophene-3-carboxylic acid, 4-(2-carboxy-4-fluorophenylthio)thiophene-3-carboxylic acid was obtained as a colorless solid in 90% yield: ¹H NMR (300 MHz, DMSO-d₆) δ 13.05 (br s, 2H), 8.43 (d, J=2.5 Hz, 1H), 7.59 (br s, 2H), 7.33-7.21 (, 1H), 6.86 (br s, 1H).

### Preparation 2.30

### Preparation of 2-(2-carboxy-4-fluorophenylthio)thiophene-3-carboxylic acid

Following the procedure as described in Preparation 2, making non-critical variations using 5-fluoro-2-mercaptobenzoic acid to replace 2-mercaptobenzoic acid to react with 2-bromothiophene-3-carboxylic acid, 2-(2-carboxy-4-fluorophenylthio)thiophene-3-carboxylic acid was obtained as a colorless solid in 91% yield: ¹H NMR (300 MHz, DMSO-d₆) δ 13.05 (br s, 2H), 8.43 (d, J=2.5 Hz, 1H), 7.70-7.47 (m, 2H), 7.33-7.19 (m, 1H), 6.95-6.74 (m, 1H).

### Preparation 2.31

### Preparation of 2-(2-carboxy-4-fluorophenylthio)-4-(methylsulfonyl)benzoic acid

Following the procedure as described in Preparation 2, making non-critical variations using 5-fluoro-2-mercaptobenzoic acid to replace 2-mercaptobenzoic acid to react with 2-chloro-4-(methylsulfonyl)benzoic acid, 2-(2-carboxy-4-fluorophenylthio)-4-(methylsulfonyl)benzoic acid was obtained as a colorless solid in 85% yield: ¹H NMR (300 MHz, DMSO-d₆) δ 13.93-13.01 (m, 2H), 8.04-7.96 (m, 1H), 7.92-7.85 (m, 1H), 7.77-7.66 (m, 1H), 7.63-7.52 (m, 1H), 7.43-7.28 (m, 2H), 3.04 (s, 3H); MS (ES−) m/z 369.1 (M−1).

### Preparation 2.32

### Preparation of 4-(4-chloro-2-methylphenylthio)-3-methylbenzonitrile

Following the procedure as described in Preparation 2, making non-critical variations using 4-chloro-2-methylthiophenol to replace 2-mercaptobenzoic acid to react with 4-bromo-3-methylbenzonitrile, 4-(4-chloro-2-methylphenylthio)-3-methylbenzonitrile was obtained as a colorless solid in 41% yield: ¹H NMR (300 MHz, DMSO-d₆) δ 7.70 (s, 1H), 7.55 (d, J=2.3 Hz, 1H), 7.49 (dd, J=2.3, 8.3 Hz, 1H), 7.42 (d, J=8.3 Hz, 1H), 7.35 (dd, J=2.3, 8.3 Hz, 1H), 6.63 (d, J=8.3 Hz, 1H), 2.33 (s, 3H), 2.24 (s, 3H).

### Preparation 3

### Preparation of dimethyl 2,2′-thiodibenzoate

To a stirred solution of 2,2′-thiodibenzoic acid (1.37 g, 5.00 mmol) in methanol (40.0 mL) was added several drops of thionyl chloride. The mixture was stirred at refluxing temperature for 2 h. Methanol was removed and the solid residue was dissolved with ether, washed with saturated sodium bicarbonate solution and water, dried over sodium sulfate and filtered. The filtrate was concentrated in vacuo. Dimethyl 2,2′-thiodibenzoate was isolated as a colorless solid in 98% yield (1.48 g). ¹H NMR (300 MHz, CDCl₃) δ 7.82 (dd, J=7.7, 1.6 Hz, 2H), 7.51-7.43 (m, 2H), 7.42-7.34 (m, 2H), 7.12 (dd, J=7.7, 1.6 Hz, 2H), 3.73 (s, 6H).

### Preparation 3.1

### Preparation of dimethyl 6,6′-thiobis(3-fluorobenzoate)

Following the procedure as described in Preparation 3, making non-critical variations using 6,6′-thiobis(3-fluorobenzoic acid) to replace 2,2′-thiodibenzoic acid, dimethyl 6,6′-thiobis(3-fluorobenzoate) was obtained as a white solid in 88% yield: ¹H NMR (300 MHz, DMSO-d₆) δ 7.66 (dd, J=9.0, 2.9 Hz, 2H), 7.39-7.31 (m, 2H), 7.17 (dd, J=9.0, 5.3 Hz, 2H), 3.75 (s, 6H).

### Preparation 3.2

### Preparation of methyl 5-fluoro-2-(2-(methoxycarbonyl)phenylthio)benzoate

Following the procedure as described in Preparation 3, making non-critical variations using 2-(2-carboxyphenylthio)-5-fluorobenzoic acid to replace 2,2′-thiodibenzoic acid, methyl 5-fluoro-2-(2-(methoxycarbonyl)phenylthio)benzoate was obtained as a colorless solid in 93% yield: MS (ES+) m/z 321.1 (M+1).

### Preparation 3.3

### Preparation of methyl 5-fluoro-2-(2-(methoxycarbonyl)-4-nitrophenylthio)benzoate

Following the procedure as described in Preparation 3, making non-critical variations using 2-(2-carboxy-4-fluorophenylthio)-5-nitrobenzoic acid to replace 2,2′-thiodibenzoic acid, methyl 5-fluoro-2-(2-(methoxycarbonyl)-4-nitrophenylthio)benzoate was obtained as a colorless solid in 87% yield: ¹H NMR (300 MHz, DMSO-d₆) δ 8.62 (d, J=2.6 Hz, 1H), 8.17 (dd, J=9.0, 2.6 Hz, 1H), 7.76 (dd, J=9.0, 2.9 Hz, 1H), 7.73 (dd, J=8.5, 5.3 Hz, 1H), 7.62-7.53 (m, 1H), 6.87 (d, J=9.0 Hz, 1H), 3.91 (s, 3H), 3.68 (s, 3H).

### Preparation 3.4

### Preparation of methyl 5-fluoro-2-(2-(methoxycarbonyl)-4-(trifluoromethyl)phenylthio)benzoate

Following the procedure as described in Preparation 3, making non-critical variations using 2-(2-carboxy-4-(trifluoromethyl)phenylthio)-5-fluorobenzoic acid to replace 2,2′-thiodibenzoic acid, methyl 5-fluoro-2-(2-(methoxycarbonyl)-4-(trifluoromethyl)phenylthio)benzoate was obtained as a colorless solid in 94% yield: ¹H NMR (300 MHz, DMSO-d₆) δ 8.14 (d, J=1.6 Hz, 1H), 7.77-7.69 (m, 2H), 7.65 (dd, J=8.7, 5.5 Hz, 1H), 7.58-7.48 (m, 1H), 6.92 (d, J=8.5 Hz, 1H), 3.86 (s, 3H), 3.68 (s, 3H).

### Preparation 3.5

### Preparation of methyl 5-amino-2-(2-(methoxycarbonyl)phenylthio)benzoate

Following the procedure as described in Preparation 3, making non-critical variations using 5-acetamido-2-(2-carboxyphenylthio)benzoic acid to replace 2,2′-thiodibenzoic acid, methyl 5-amino-2-(2-(methoxycarbonyl)phenylthio)benzoate was obtained as a colorless solid in 86% yield: ¹H NMR (300 MHz, DMSO-d₆) δ 7.83 (dd, J=7.8, 1.5 Hz, 1H), 7.32 (ddd, J=8.5, 7.3, 1.5 Hz, 1H), 7.20-7.08 (m, 2H), 6.91 (d, J=2.6 Hz, 1H), 6.71 (dd, J=8.4, 2.6 Hz, 1H), 6.67 (dd, J=8.2, 0.9 Hz, 1H), 5.83 (s, 2H), 3.82 (s, 3H), 3.59 (s, 3H).

### Preparation 4

### Preparation of 1-(hydroxymethyl)-2-((6-(hydroxymethyl)phenyl)thio)benzene

To a stirred solution of dimethyl 2,2′-thiodibenzoate (1.20 g, 3.97 mmol) in a mixture of ether/tetrahydrofuran (1/1, 40.0 mL) was added lithium aluminum hydride (0.68 g). The mixture was stirred for 16 h at ambient temperature. Saturated sodium sulfate solution was added and the mixture was extracted with ethyl ether. The organic layer was dried over sodium sulfate, filtered and concentrated to afford 1-(hydroxymethyl)-2-((6-(hydroxymethyl)phenyl)thio)benzene as an oil in 97% yield (0.95 g). ¹H NMR (300 MHz, CDCl₃) δ 7.49-7.43 (m, 2H), 7.32-7.09 (m, 6H), 4.74 (s, 4H), 2.15 (s, 2H).

### Preparation 4.1

### Preparation of 5-fluoro-1-(hydroxymethyl)-2-((4-fluoro-6-(hydroxymethyl)phenyl)thio)benzene

Following the procedure as described in Preparation 4, making non-critical variations using dimethyl 6,6′-thiobis(3-fluorobenzoate) to replace dimethyl 2,2′-thiodibenzoate, 5-fluoro-1-(hydroxymethyl)-2-((4-fluoro-6-(hydroxymethyl)phenyl)thio)benzene was obtained as a colorless solid in 84% yield: ¹H NMR (300 MHz, CDCl₃) δ 7.25 (dd, J=2.9, 9.3 Hz, 2H), 7.08 (dd, J=5.5, 8.6 Hz, 2H), 6.95-6.85 (m, 2H), 4.71 (s, 4H).

### Preparation 4.2

### Preparation of (5-fluoro-2-(2-(hydroxymethyl)phenylthio)phenyl)methanol

Following the procedure as described in Preparation 4, making non-critical variations using methyl 5-fluoro-2-(2-(methoxycarbonyl)phenylthio)benzoate to replace dimethyl 2,2′-thiodibenzoate, (5-fluoro-2-(2-(hydroxymethyl)phenylthio)phenyl)-methanol was obtained as a colorless solid in 88% yield: ¹H NMR (300 MHz, CDCl₃) δ 7.42 (dd, J=7.4, 1.4 Hz, 1H), 7.30-7.21 (m, 3H), 7.17 (ddd, J=9.0, 7.1, 1.4 Hz, 1H), 6.94 (dd, J=7.7, 1.0 Hz, 2H), 4.76 (s, 2H), 4.69 (s, 2H).

### Preparation 4.3

### Preparation of (2-(2-(hydroxymethyl)-5-methylphenylthio)phenyl)methanol

Following the procedure as described in Preparation 4, making non-critical variations using 2-(2-carboxyphenylthio)-4-methylbenzoic acid to replace dimethyl 2,2′-thiodibenzoate, (2-(2-(hydroxymethyl)-5-methylphenylthio)phenyl)methanol was obtained as a colorless solid in 80% yield: ¹H NMR (300 MHz, CDCl₃) δ 7.50 (d, J=7.5 Hz, 1H), 7.41 (d, J=7.7 Hz, 1H), 7.26 (t, J=7.0 Hz, 1H), 7.14 (dd, J=11.0, 7.8 Hz, 1H), 6.93 (dd, J=7.7, 1.3 Hz, 1H), 6.89 (s, 1H), 5.31 (t, J=5.1 Hz, 1H), 5.23 (t, J=5.1 Hz, 1H), 4.52 (d, J=5.1 Hz, 2H), 4.46 (d, J=5.1 Hz, 2H), 2.16 (s, 3H).

### Preparation 4.4

### Preparation of (2-(2-(hydroxymethyl)-4-methoxyphenylthio)phenyl)methanol

Following the procedure as described in Preparation 4, making non-critical variations using 2-(2-carboxyphenylthio)-5-methoxybenzoic acid to replace dimethyl 2,2′-thiodibenzoate, (2-(2-(hydroxymethyl)-4-methoxyphenylthio)phenyl)methanol was obtained as a colorless solid in 85% yield: MS (ES+) m/z 259.1 (M−17).

### Preparation 4.5

### Preparation of (5-fluoro-2-(2-(hydroxymethyl)-5-methylphenylthio)phenyl)methanol

Following the procedure as described in Preparation 4, making non-critical variations using 2-(2-carboxy-4-fluorophenylthio)-4-methylbenzoic acid to replace dimethyl 2,2′-thiodibenzoate, (5-fluoro-2-(2-(hydroxymethyl)-5-methylphenylthio)phenyl)methanol was obtained as a colorless solid in 76% yield: MS (ES+) m/z 261.1 (M−17).

### Preparation 4.6

### Preparation of (5-fluoro-2-(2-(hydroxymethyl)-4-methoxyphenylthio)phenyl)methanol

Following the procedure as described in Preparation 4, making non-critical variations using 2-(2-carboxy-4-fluorophenylthio)-5-methoxybenzoic acid to replace dimethyl 2,2′-thiodibenzoate, (5-fluoro-2-(2-(hydroxymethyl)-4-methoxyphenylthio)phenyl)methanol was obtained as a colorless solid in 88% yield: MS (ES+) m/z 277.1 (M−17).

### Preparation 4.7

### Preparation of (4-chloro-2-(4-fluoro-2-(hydroxymethyl)phenylthio)phenyl)methanol

Following the procedure as described in Preparation 4, making non-critical variations using 2-(2-carboxy-4-fluorophenylthio)-4-chlorobenzoic acid to replace dimethyl 2,2′-thiodibenzoate, (4-chloro-2-(4-fluoro-2-(hydroxymethyl)phenylthio)phenyl)methanol was obtained as a colorless solid in 98% yield: MS (ES+) m/z 281.1 (M−17), 283.1 (M−17).

### Preparation 4.8

### Preparation of (2-(2-(hydroxymethyl)-6-methylphenylthio)phenyl)methanol

Following the procedure as described in Preparation 4, making non-critical variations using 2-(2-carboxy-6-methylphenylthio)benzoic acid to replace dimethyl 2,2′-thiodibenzoate, (2-(2-(hydroxymethyl)-6-methylphenylthio)phenyl)methanol was obtained as a colorless solid in 88% yield: MS (ES+) m/z 243.1 (M−17).

### Preparation 4.9

### Preparation of (4,5-difluoro-2-(2-(hydroxymethyl)phenylthio)phenyl)methanol

Following the procedure as described in Preparation 4, making non-critical variations using 2-(2-carboxyphenylthio)-4,5-difluorobenzoic acid to replace dimethyl 2,2′-thiodibenzoate, (4,5-difluoro-2-(2-(hydroxymethyl)phenylthio)phenyl)methanol was obtained as a colorless solid in 74% yield: MS (ES+) m/z 265.1 (M−17).

### Preparation 4.10

### Preparation of (2-(2-(hydroxymethyl)-4-methylphenylthio)phenyl)methanol

Following the procedure as described in Preparation 4, making non-critical variations using 2-(2-carboxy-5-methylphenylthio)benzoic acid to replace dimethyl 2,2′-thiodibenzoate, (2-(2-(hydroxymethyl)-4-methylphenylthio)phenyl)methanol was obtained as a colorless solid in 74% yield: MS (ES+) m/z 243.1 (M−17).

### Preparation 4.11

### Preparation of (4-chloro-2-(2-(hydroxymethyl)phenylthio)phenyl)methanol

Following the procedure as described in Preparation 4, making non-critical variations using 2-(2-carboxyphenylthio)-4-chlorobenzoic acid to replace dimethyl 2,2′-thiodibenzoate, (4-chloro-2-(2-(hydroxymethyl)phenylthio)phenyl)methanol was obtained as a colorless solid in 79% yield: MS (ES+) m/z 263.1 (M−17), 265.1 (M−17).

### Preparation 4.12

### Preparation of (4-fluoro-2-(2-(hydroxymethyl)phenylthio)phenyl)methanol

Following the procedure as described in Preparation 4, making non-critical variations using 2-(2-carboxyphenylthio)-4-fluorobenzoic acid to replace dimethyl 2,2′-thiodibenzoate, (4-fluoro-2-(2-(hydroxymethyl)phenylthio)phenyl)methanol was obtained as a colorless solid in 75% yield: MS (ES+) m/z 247.1 (M−17).

### Preparation 4.13

### Preparation of (2-(1-(hydroxymethyl)naphthalen-2-ylthio)phenyl)methanol

Following the procedure as described in Preparation 4, making non-critical variations using 2-(2-carboxyphenylthio)-1-naphthoic acid to replace dimethyl 2,2′-thiodibenzoate, (2-(1-(hydroxymethyl)naphthalen-2-ylthio)phenyl)methanol was obtained as a colorless solid in 78% yield: MS (ES+) m/z 279.1 (M−17).

### Preparation 4.14

### Preparation of (5-fluoro-2-(1-(hydroxymethyl)naphthalen-2-ylthio)phenyl)methanol

Following the procedure as described in Preparation 4, making non-critical variations using 2-(2-carboxy-4-fluorophenylthio)-1-naphthoic acid to replace dimethyl 2,2′-thiodibenzoate, (5-fluoro-2-(1-(hydroxymethyl)naphthalen-2-ylthio)phenyl)methanol was obtained as a colorless solid in 81% yield: MS (ES+) m/z 297.1 (M−17).

### Preparation 4.15

### Preparation of (4-fluoro-2-(4-fluoro-2-(hydroxymethyl)phenylthio)phenyl)methanol

Following the procedure as described in Preparation 4, making non-critical variations using 2-(2-carboxy-4-fluorophenylthio)-4-fluorobenzoic acid to replace dimethyl 2,2′-thiodibenzoate, (4-fluoro-2-(4-fluoro-2-(hydroxymethyl)phenylthio)phenyl)methanol was obtained as a colorless solid in 89% yield: MS (ES+) m/z 265.1 (M−17).

### Preparation 4.16

### Preparation of (4,5-difluoro-2-(4-fluoro-2-(hydroxymethyl)phenylthio)phenyl)methanol

Following the procedure as described in Preparation 4, making non-critical variations using 2-(2-carboxy-4-fluorophenylthio)-4,5-difluorobenzoic acid to replace dimethyl 2,2′-thiodibenzoate, (4,5-difluoro-2-(4-fluoro-2-(hydroxymethyl)phenylthio)phenyl)methanol was obtained as a colorless solid in 79% yield: MS (ES+) m/z 283.1 (M−17).

### Preparation 4.17

### Preparation of (5-fluoro-2-(2-(hydroxymethyl)-4-(trifluoromethyl)phenylthio)phenyl)methanol

Following the procedure as described in Preparation 4, making non-critical variations using methyl 5-fluoro-2-(2-(methoxycarbonyl)-4-(trifluoromethyl)-phenylthio)benzoate to replace dimethyl 2,2′-thiodibenzoate, (5-fluoro-2-(2-(hydroxymethyl)-4-(trifluoromethyl)phenylthio)phenyl)methanol was obtained as a colorless solid in 98% yield: ¹H NMR (300 MHz, DMSO-d₆) δ 7.74 (d, J=0.9 Hz, 1H), 7.51-7.42 (m, 2H), 7.39 (dd, J=10.1, 2.9 Hz, 1H), 7.25-7.14 (m, 1H), 6.75 (d, J=8.2 Hz, 1H), 5.60 (t, J=5.4 Hz, 1H), 5.46 (t, J=5.4 Hz, 1H), 4.58 (d, J=5.4 Hz, 2H), 4.44 (d, J=5.4 Hz, 2H).

### Preparation 4.18

### Preparation of (5-amino-2-(2-(hydroxymethyl)phenylthio)phenyl)methanol

Following the procedure as described in Preparation 4, making non-critical variations using methyl 5-amino-2-(2-(methoxycarbonyl)phenylthio)benzoate to replace dimethyl 2,2′-thiodibenzoate, (5-amino-2-(2-(hydroxymethyl)phenylthio)phenyl)-methanol was obtained as a colorless solid in 91% yield: MS (ES+) m/z 262.1 (M+1).

### Preparation 4.19

### Preparation of 1-(hydroxymethyl)-2-((6-hydroxymethylphenyl)(methyl)amino)benzene

Following the procedure as described in Preparation 4, making non-critical variations using dimethyl 2,2′-(methylazanediyl)dibenzoate to replace dimethyl 2,2′-thiodibenzoate, 1-(hydroxymethyl)-2-((6-hydroxymethylphenyl)(methyl)amino)benzene was obtained as a colorless oil in 88% yield: ¹H NMR (300 MHz, DMSO-d₆) δ 7.48-7.40 (m, 2H), 7.19-7.09 (m, 2H), 7.08-6.99 (m, 2H), 6.88-6.80 (m, 2H), 5.04 (t, J=5.5 Hz, 2H), 4.17 (d, J=5.5 Hz, 4H), 3.00 (s, 3H).

### Preparation 5

### Preparation of (5-fluoro-2-(2-(hydroxymethyl)-4-nitrophenylthio)phenyl)methanol

Methyl 5-fluoro-2-(2-(methoxycarbonyl)-4-nitrophenylthio)benzoate (0.94 g, 2.57 mmol) was added to a suspension of sodium borohydride (0.39 g, 10.30 mmol) in tetrahydrofuran (30 mL) at 70° C. The mixture was stirred for 15 min. Methanol (5 mL) was added dropwise. The mixture was cooled to ambient temperature, followed by the addition of saturated aqueous ammonium chloride (10 mL). The mixture was stirred for 1.5 h. The organic layer was separated and the aqueous phase was extracted with ethyl acetate. The combined organic solution was dried over sodium sulfate, filtered and concentrated in vacuo to afford (5-fluoro-2-(2-(hydroxymethyl)-4-nitrophenylthio)phenyl)methanol as a colorless solid in 88% yield (0.71 g): ¹H NMR (300 MHz, DMSO-d₆) δ 8.26 (d, J=2.5 Hz, 1H), 7.92 (dd, J=8.7, 2.5 Hz, 1H), 7.55 (dd, J=8.7, 5.7 Hz, 1H), 7.42 (dd, J=10.0, 2.8 Hz, 1H), 7.27-7.17 (m, 1H), 6.67 (d, J=8.7 Hz, 1H), 5.44 (br s, 2H), 4.61 (s, 2H), 4.44 (s, 2H).

### Preparation 6

### Preparation of (5-fluoro-2-(2-(hydroxymethyl)-6-nitrophenylthio)phenyl)methanol

To a stirred solution of 2-(2-carboxy-6-nitrophenylthio)-5-fluorobenzoic acid (1.45 g, 4.30 mmol) in tetrahydrofuran (55 mL) was added a borane tetrahydrofuran complex solution (14.00 mL of 1 M solution in tetrahydrofuran, 14.00 mmol). The mixture was stirred at ambient temperature overnight, and followed by the addition of methanol (15 mL). The solvent was removed in vacuo and the residue was dissolved in ethyl acetate (75 mL). This solution was washed with water and sodium bicarbonate, dried over sodium sulfate, filtered and concentrated in vacuo to afford (5-fluoro-2-(2-(hydroxymethyl)-6-nitrophenylthio)phenyl)methanol as a dark oil in 73% yield (1.02 g): ¹H NMR (300 MHz, DMSO-d₆) δ 7.85-7.80 (m, 2H), 7.72-7.63 (m, 1H), 7.24 (dd, J=9.9, 2.8 Hz, 1H), 7.03-6.94 (m, 1H), 6.73 (dd, J=8.6, 5.5 Hz, 1H), 5.53 (t, J=5.5 Hz, 2H), 4.49 (d, J=5.5 Hz, 2H), 4.39 (d, J=5.5 Hz, 2H).

### Preparation 6.1

### Preparation of (5-chloro-2-(4-fluoro-2-(hydroxymethyl)phenylthio)phenyl)methanol

Following the procedure as described in Preparation 6, making non-critical variations using 2-(2-carboxy-4-chlorophenylthio)-5-fluorobenzoic acid to replace 2-(2-carboxy-6-nitrophenylthio)-5-fluorobenzoic acid to react with borane, (5-chloro-2-(4-fluoro-2-(hydroxymethyl)phenylthio)phenyl)methanol was obtained as a colorless oil in 51% yield: MS (ES+) m/z 281.1 (M−17), 283.1 (M−17).

### Preparation 6.2

### Preparation of (5-(ethylamino)-2-(2-(hydroxymethyl)phenylthio)phenyl)methanol

Following the procedure as described in Preparation 6, making non-critical variations using 5-acetamido-2-(2-carboxyphenylthio)benzoic acid to replace 2-(2-carboxy-6-nitrophenylthio)-5-fluorobenzoic acid to react with borane, (5-(ethylamino)-2-(2-(hydroxymethyl)phenylthio)phenyl)methanol was obtained as a colorless oil in 52% yield: ¹H NMR (300 MHz, DMSO-d₆) δ 7.41-7.36 (m, 1H), 7.11 (d, J=8.3 Hz, 1H), 7.09-6.98 (m, 2H), 6.83 (d, J=2.5 Hz, 1H), 6.53 (dd, J=7.5, 1.5 Hz, 1H), 6.44 (dd, J=8.4, 2.5 Hz, 1H), 6.00 (s, 1H), 5.24 (s, 1H), 5.06 (s, 1H), 4.52 (s, 2H), 4.35 (s, 2H), 3.11-2.91 (m, 2H), 1.14 (t, J=7.1 Hz, 3H).

### Preparation 6.3

### Preparation of 5-chloro-1-(hydroxymethyl)-2-((4-chloro-6-(hydroxymethyl)phenyl)thio)benzene

Following the procedure as described in Preparation 6, making non-critical variations using 6,6′-thiobis(3-chlorobenzoic acid) to replace 2-(2-carboxy-6-nitrophenylthio)-5-fluorobenzoic acid to react with borane, 5-chloro-1-(hydroxymethyl)-2-((4-chloro-6-(hydroxymethyl)phenyl)thio)benzene was obtained as a colorless solid in 98% yield: ¹H NMR (300 MHz, DMSO-d₆) δ 7.52 (d, J=2.5 Hz, 2H), 7.27 (dd, J=8.3, 2.5 Hz, 2H), 7.02 (d, J=8.3 Hz, 2H), 5.46 (t, J=5.5 Hz, 2H), 4.47 (d, J=5.5 Hz, 4H).

### Preparation 6.4

### Preparation of 2-(5-fluoro-2-{[4-fluoro-2-(hydroxymethyl)-phenyl]thio}phenyl)ethanol

Following the procedure as described in Preparation 6, making non-critical variations using 2-(2-(carboxymethyl)-4-fluorophenylthio)-5-fluorobenzoic acid to replace 2-(2-carboxy-6-nitrophenylthio)-5-fluorobenzoic acid to react with borane, 2-(5-fluoro-2-{[4-fluoro-2-(hydroxymethyl)-phenyl]thio}phenyl)ethanol was obtained as a colorless solid in 86% yield: ¹H NMR (300 MHz, DMSO-d₆) δ 7.29 (d, J=10.2 Hz, 1H), 7.19 (dd, J=10.2, 1.8 Hz, 1H), 7.06 (dd, J=6.8, 1.8 Hz, 2H), 7.00 (s, 2H), 5.47 (s, 1H), 4.73 (s, 1H), 4.48 (s, 2H), 3.56 (t, J=6.8 Hz, 2H), 2.83 (t, J=6.8 Hz, 2H).

### Preparation 6.5

### Preparation of 2-(5-chloro-2-(4-fluoro-2-(hydroxymethyl)phenylthio)phenyl)ethanol

Following the procedure as described in Preparation 6, making non-critical variations using 2-(2-(carboxymethyl)-4-chlorophenylthio)-5-fluorobenzoic acid to replace 2-(2-carboxy-6-nitrophenylthio)-5-fluorobenzoic acid to react with borane, 2-(5-chloro-2-(4-fluoro-2-(hydroxymethyl)phenylthio)phenyl)ethanol was obtained as a colorless solid in 86% yield: ¹H NMR (300 MHz, DMSO-d₆) δ 7.35 (d, J=2.3 Hz, 1H), 7.31 (d, J=2.3 Hz, 1H), 7.25 (dd, J=8.5, 5.7 Hz, 1H), 7.17 (dd, J=8.5, 2.3 Hz, 1H), 7.19-7.06 (m, 1H), 6.75 (d, J=8.5 Hz, 1H), 5.46 (s, 1H), 4.76 (s, 1H), 4.45 (s, 2H), 3.59 (t, J=6.8 Hz, 2H), 2.83 (t, J=6.8 Hz, 2H).

### Preparation 6.6

### Preparation of 2-(2-(2-(hydroxymethyl)phenylthio)phenyl)ethanol

Following the procedure as described in Preparation 6, making non-critical variations using 2-(2-(carboxymethyl)phenylthio)benzoic acid to replace 2-(2-carboxy-6-nitrophenylthio)-5-fluorobenzoic acid to react with borane, 2-(2-(2-(hydroxymethyl)phenylthio)phenyl)ethanol was obtained as a colorless oil in 89% yield: MS (ES+) m/z 243.1 (M−17).

### Preparation 6.7

### Preparation of (5-chloro-2-(2-(hydroxymethyl)phenylthio)phenyl)methanol

Following the procedure as described in Preparation 6, making non-critical variations using 2-[(2-carboxyphenyl)thio]-5-chlorobenzoic acid to replace 2-(2-carboxy-6-nitrophenylthio)-5-fluorobenzoic acid to react with borane, (5-chloro-2-(2-(hydroxymethyl)phenylthio)phenyl)methanol was obtained as a colorless solid in 85% yield: ¹H NMR (300 MHz, DMSO-d₆) δ 7.60-7.50 (m, 2H), 7.36-7.29 (m, 1H), 7.22 (ddd, J=8.9, 7.9, 2.0 Hz, 2H), 7.05 (dd, J=7.7, 1.1 Hz, 1H), 6.96 (d, J=8.3 Hz, 1H), 5.44 (br s, 1H), 5.28 (br s, 1H), 4.48 (d, J=3.9 Hz, 4H).

### Preparation 6.8

### Preparation of (2-(2-(hydroxymethyl)-4-(trifluoromethyl)phenylthio)phenyl)methanol

Following the procedure as described in Preparation 6, making non-critical variations using 2-[(2-carboxyphenyl)thio]-5-(trifluoromethyl)benzoic acid to replace 2-(2-carboxy-6-nitrophenylthio)-5-fluorobenzoic acid to react with borane, (2-(2-(hydroxymethyl)-4-(trifluoromethyl)phenylthio)phenyl)methanol was obtained as a colorless solid in 83% yield: ¹H NMR (300 MHz, DMSO-d₆) δ 7.76 (d, J=1.4 Hz, 1H), 7.61 (d, J=8.0 Hz, 1H), 7.50-7.45 (m, 2H), 7.35-7.30 (m, 2H), 6.84 (d, J=8.0 Hz, 1H), 5.58 (s, 1H), 5.29 (s, 1H), 4.58 (s, 2H), 4.48 (s, 2H).

### Preparation 6.9

### Preparation of (5-fluoro-2-(2-(hydroxymethyl)-4-(methylsulfonyl)phenylthio)phenyl)methanol

Following the procedure as described in Preparation 6, making non-critical variations using 2-(2-carboxy-4-(methylsulfonyl)phenylthio)-5-fluorobenzoic acid to replace 2-(2-carboxy-6-nitrophenylthio)-5-fluorobenzoic acid to react with borane, (5-fluoro-2-(2-(hydroxymethyl)-4-(methylsulfonyl)phenylthio)phenyl)methanol was obtained as a colorless solid in 80% yield: ¹H NMR (300 MHz, DMSO-d₆) δ 7.96 (d, J=2.0 Hz, 1H), 7.61 (dd, J=8.3, 2.0 Hz, 1H), 7.51 (dd, J=8.3, 5.7 Hz, 1H), 7.41 (dd, J=10.1, 3.0 Hz, 1H), 7.25-7.16 (m, 1H), 6.73 (d, J=8.3 Hz, 1H), 5.64 (t, J=5.0 Hz, 1H), 5.47 (t, J=5.7, 5.0 Hz, 1H), 4.60 (d, J=3.0 Hz, 2H), 4.44 (d, J=3.0 Hz, 2H), 3.13 (s, 3H).

### Preparation 6.10

### Preparation of (4-(4-fluoro)-2-(hydroxymethyl)phenylthio)-3-(hydroxymethyl)-N,N-dimethylbenzenesulfonamide

Following the procedure as described in Preparation 6, making non-critical variations using 2-(2-carboxy-4-(N,N-dimethylsulfamoyl)phenylthio)-5-fluorobenzoic acid to replace 2-(2-carboxy-6-nitrophenylthio)-5-fluorobenzoic acid to react with borane, (4-(4-fluoro)-2-(hydroxymethyl)phenylthio)-3-(hydroxymethyl)-N,N-dimethylbenzenesulfonamide was obtained as a viscous oil in 88% yield: MS (ES+) m/z 355.1 (M−17).

### Preparation 6.11

### Preparation of 1-(hydroxymethyl)-2-((6-hydroxymethylphenyl)sulfonyl)benzene

Following the procedure as described in Preparation 6, making non-critical variations using 2,2′-sulfonyldibenzoic acid to replace 2-(2-carboxy-6-nitrophenylthio)-5-fluorobenzoic acid to react with borane, 1-(hydroxymethyl)-2-((6-hydroxymethylphenyl)sulfonyl)benzene was obtained as a colorless solid in 88% yield: ¹H NMR (300 MHz, DMSO-d₆) δ 8.02-7.92 (m, 2H), 7.81-7.68 (m, 4H), 7.60-7.49 (m, 2H), 5.39 (t, J=5.66 Hz, 1H), 4.50 (d, J=5.42 Hz, 4H).

### Preparation 6.12

### Preparation of 5-fluoro-1-(hydroxymethyl)-2-((4-fluoro-6-hydroxymethylphenyl)sulfonyl)benzene

Following the procedure as described in Preparation 6, making non-critical variations using 6,6′-sulfonylbis(3-fluorobenzoic acid) to replace 2-(2-carboxy-6-nitrophenylthio)-5-fluorobenzoic acid to react with borane, 5-fluoro-1-(hydroxymethyl)-2-((4-fluoro-6-hydroxymethylphenyl)sulfonyl)benzene was obtained as a colorless solid used for the next step reaction without purification. MS (ES+) m/z 297.1 (M−17).

### Preparation 6.13

### Preparation of 1-(2-hydroxyethyl)-2-((6-(2-hydroxyethyl)phenyl)thio)benzene

Following the procedure as described in Preparation 6, making non-critical variations using 2-(2-((6-carboxymethylphenyl)thio)phenyl)acetic acid to replace 2-(2-carboxy-6-nitrophenylthio)-5-fluorobenzoic acid to react with borane, 1-(2-hydroxyethyl)-2-((6-(2-hydroxyethyl)phenyl)thio)benzene was obtained as a viscous oil in 73% yield: ¹H NMR (300 MHz, DMSO-d₆) δ 7.30 (dd, J=7.5, 1.3 Hz, 2H), 7.24-7.17 (m, 2H), 7.16-7.09 (m, 2H), 6.94 (dd, J=7.5, 1.3 Hz, 2H), 4.67 (br s, 2H), 3.56 (t, J=7.2 Hz, 4H), 2.84 (t, J=7.2 Hz, 4H).

### Preparation 6.14

### Preparation of (5-fluoro-2-(4-(hydroxymethyl)thiophen-3-ylthio)phenyl)methanol

Following the procedure as described in Preparation 6, making non-critical variations using 4-(2-carboxy-4-fluorophenylthio)thiophene-3-carboxylic acid to replace 2-(2-carboxy-6-nitrophenylthio)-5-fluorobenzoic acid to react with borane, (5-fluoro-2-(4-(hydroxymethyl)thiophen-3-ylthio)phenyl)methanol was obtained as a viscous oil in 72% yield: ¹H NMR (300 MHz, DMSO-d₆) δ 7.68 (d, J=5.4 Hz, 1H), 7.23 (dd, J=9.9, 2.9 Hz, 1H), 7.19 (d, J=5.5 Hz, 1H), 7.06-6.96 (m, 1H), 6.89 (dd, J=8.6, 5.5 Hz, 1H), 5.48 (s, 1H), 5.20 (s, 1H), 4.58 (s, 2H), 4.42 (s, 2H).

### Preparation 6.15

### Preparation of (5-fluoro-2-(3-(hydroxymethyl)thiophen-2-ylthio)phenyl)methanol

Following the procedure as described in Preparation 6, making non-critical variations using 2-(2-carboxy-4-fluorophenylthio)thiophene-3-carboxylic acid to replace 2-(2-carboxy-6-nitrophenylthio)-5-fluorobenzoic acid to react with borane, (5-fluoro-2-(3-(hydroxymethyl)thiophen-2-ylthio)phenyl)methanol was obtained as a viscous oil in 81% yield: ¹H NMR (300 MHz, DMSO-d₆) δ 7.52 (d, J=3.3 Hz, 1H), 7.47-7.43 (m, 1H), 7.25 (dd, J=2.8, 8.5 Hz, 1H), 7.05-6.96 (m, 1H), 6.92 (dd, J=5.5, 8.5 Hz, 1H), 4.53 (s, 2H), 4.38 (br s, 2H), 4.25 (d, J=1.0 Hz, 2H).

### Preparation 6.16

### Preparation of (5-fluoro-2-(2-(hydroxymethyl)-5-(methylsulfonyl)phenylthio)phenyl)methanol

Following the procedure as described in Preparation 6, making non-critical variations using 2-(2-carboxy-4-fluorophenylthio)-4-(methylsulfonyl)benzoic acid to replace 2-(2-carboxy-6-nitrophenylthio)-5-fluorobenzoic acid to react with borane, (5-fluoro-2-(2-(hydroxymethyl)-5-(methylsulfonyl)phenylthio)phenyl)methanol was obtained as a viscous oil in 44% yield: MS (ES+) m/z 325.1 (M−17).

### Preparation 7

### Preparation of bis(2-(bromomethyl)-4-fluorophenyl)sulfane

To a stirred solution of (2-(2-(hydroxymethyl)-4-fluorophenylthio)-5-fluorophenyl)methanol (0.71 g, 2.51 mmol) in dry ether (40.0 mL) was added phosphorus tribromide (2.04 g, 7.52 mmol) in one portion. The mixture was stirred at ambient temperature for 16 h, washed with water and dried over magnesium sulfate and filtered. The filtrate was concentrated in vacuo to afford bis(2-(bromomethyl)-4-fluorophenyl)sulfane as a colorless solid in 95% yield (1.00 g): ¹H NMR (300 MHz, CDCl₃) δ 7.19 (dd, J=9.2, 2.3 Hz, 2H), 7.13 (dd, J=8.7, 5.5 Hz, 2H), 6.97-6.89 (m, 2H), 4.62 (s, 4H).

### Preparation 7.1

### Preparation of (2-(bromomethyl)-4-fluorophenyl)(2-(bromomethyl)-4-nitrophenyl)sulfane

Following the procedure as described in Preparation 7, making non-critical variations using (5-fluoro-2-(2-(hydroxymethyl)-4-nitrophenylthio)phenyl)methanol to replace (2-(2-(hydroxymethyl)-4-fluorophenylthio)-5-fluorophenyl)methanol to react with phosphorus tribromide, (2-(bromomethyl)-4-fluorophenyl)(2-(bromomethyl)-4-nitrophenyl)sulfane was obtained as a colorless solid in 66% yield: ¹H NMR (300 MHz, CDCl₃) δ 7.22-7.16 (m, 2H), 7.16-7.09 (m, 2H), 6.98-6.88 (m, 2H), 4.62 (s, 4H).

### Preparation 7.2

### Preparation of 3-bromomethyl-4-(2-bromomethyl-4-fluorophenylthio)-N,N-dimethylbenzenesulfonamide

Following the procedure as described in Preparation 7, making non-critical variations using 4-(4-fluoro-2-(hydroxymethyl)phenylthio)-3-(hydroxymethyl)-N,N-dimethylbenzenesulfonamide to replace (2-(2-(hydroxymethyl)-4-fluorophenylthio)-5-fluorophenyl)methanol to react with phosphorus tribromide, 3-bromomethyl-4-(2-bromomethyl-4-fluorophenylthio)-N,N-dimethylbenzenesulfonamide was obtained as a colorless solid in 66% yield. MS (ES+) m/z 498.1 (M+1)

### Preparation 7.3

### Preparation of 2,2′-sulfonylbis((bromomethyl)benzene)

Following the procedure as described in Preparation 4, making non-critical variations using 1-(hydroxymethyl)-2-((6-hydroxymethylphenyl)sulfonyl)benzene to replace (2-(2-(hydroxymethyl)-4-fluorophenylthio)-5-fluorophenyl)methanol to react with phosphorus tribromide, 2,2′-sulfonylbis((bromomethyl)benzene) was obtained as a light pink solid in 47% yield: ¹H NMR (300 MHz, DMSO-d₆) δ 8.04 (dd, J=7.9, 1.1 Hz, 2H), 7.77-7.72 (m, 2H), 7.67-7.60 (m, 4H), 4.83 (s, 4H).

### Preparation 7.4

### Preparation of 4,4′-sulfonylbis(3-(bromomethyl)-1-fluorobenzene)

Following the procedure as described in Preparation 7, making non-critical variations using 5-fluoro-1-(hydroxymethyl)-2-((4-fluoro-6-hydroxymethylphenyl)sulfonyl)benzene to replace (2-(2-(hydroxymethyl)-4-fluorophenylthio)-5-fluorophenyl)methanol to react with phosphorus tribromide, 4,4′-sulfonylbis(3-(bromomethyl)-1-fluorobenzene) was obtained as a colorless solid in 21% yield: ¹H NMR (300 MHz, DMSO-d₆) δ 8.17 (dd, J=5.6, 8.7 Hz, 2H), 7.58 (dd, J=2.7, 9.6 Hz, 2H), 7.50 (ddd, J=2.7, 8.2, 8.7 Hz, 2H), 4.78 (s, 4H).

### Preparation 8

### Preparation of dimethyl 2,2′-(methylazanediyl)dibenzoate

Methyl N-methylanthranilate (5.00 g, 30.27 mmol) and methyl 2-iodobenzoate (7.62 g, 29.09 mmol) were dissolved in dibutylether (50 mL). Potassium carbonate (2.80 g, 20.0 mmol), copper powder (0.53 g, 8.34 mmol) and copper iodide (0.42 g, 2.21 mmol) were added. The mixture was heated at reflux for 70 h, cooled and filtered. The filtrate was concentrated in vacuo and the residue was dissolved in ether, washed with water, dried and filtered. The filtrate was concentrated in vacuo and the residue was purified by flash column chromatography to afford dimethyl 2,2′-(methylazanediyl)dibenzoate was obtained as a colorless solid in 15% yield (1.32 g): ¹H NMR (300 MHz, DMSO-d₆) δ 7.21 (ddd, J=8.3, 7.5, 1.7 Hz, 2H), 7.16 (dd, J=7.5, 1.7 Hz, 2H), 6.88 (dd, J=8.3, 1.0 Hz, 2H), 6.81-6.73 (m, 2H), 3.06 (s, 6H), 3.01 (s, 3H).

### Preparation 9

### Preparation of 2,2′-sulfonyldibenzoic acid

2,2′-Thiodibenzoic acid (0.55 g, 2.00 mmol) was mixed with sodium periodate (1.30 g, 6.00 mmol) in water (15 mL). The mixture was heated at 140° C. in an oil bath for 3 hours and another portion of sodium periodate (0.40 g, 6.00 mmol) was added. The mixture was heated for 2 h, concentrated in vacuo to dryness. The residue was extracted with hot methanol. The combined extract was concentrated in vacuo to afford 2,2′-sulfonyldibenzoic acid as a colorless solid in 98% yield (0.60 g): ¹H NMR (300 MHz, DMSO-d₆) δ 13.56 (s, 2H), 8.00 (dd, J=1.2, 7.7 Hz, 2H), 7.75-7.60 (m, 6H).

### Preparation 9.1

### Preparation of 6,6′-sulfonylbis(3-fluorobenzoic acid)

Following the procedure as described in Preparation 9, making non-critical variations using 6,6′-thiobis(3-fluorobenzoic acid) to replace 2,2′-thiodibenzoic acid, 6,6′-sulfonylbis(3-fluorobenzoic acid) was obtained as a colorless solid in 95% yield: ¹H NMR (300 MHz, DMSO-d₆) δ 13.76 (s, 2H), 8.11-8.03 (m, 2H), 7.58-7.49 (m, 4H).

### Preparation 10

### Preparation of 2-(2-((6-carboxymethylphenyl)thio)phenyl)acetic acid

2-(2-((6-cyanomethylphenyl)thio)phenyl)acetonitrile (2.00 g, 7.57 mmol) was stirred at reflux in aqueous sodium hydroxide solution (1.21 g, 30.3 mmol, 40 mL) for 7 h. The mixture was acidified with concentrated hydrochloric acid and the solid obtained was collected by filtration and dried to afford 2-(2-((6-carboxymethylphenyl)thio)-phenyl)acetic acid as a colorless solid in 95% yield (2.17 g): ¹H NMR (300 MHz, DMSO-d₆) δ 12.36 (s, 2H), 7.31 (dd, J=1.5, 7.4 Hz, 2H), 7.25-7.15 (m, 4H), 7.03 (dd, J=1.5, 7.4 Hz, 2H), 3.69 (s, 4H).

### Preparation 11

### Preparation of 3-(chloromethyl)-4-(2-(chloromethyl)-4-fluorophenylthio)thiophene

To a stirred solution of (5-fluoro-2-(4-(hydroxymethyl)thiophen-3-ylthio)phenyl)methanol (0.64 g, 2.37 mmol) in dichloromethane (20 mL) was added thionyl chloride (2.80 mL, 23.00 mmol) and the mixture was maintained at ambient temperature for 16 h and washed with water. The organic layer was separated and dried over sodium sulfate and filtered. The filtrate was concentrated in vacuo to dryness to afford 3-(chloromethyl)-4-(2-(chloromethyl)-4-fluorophenylthio)thiophene as a pink oil in 98% yield: ¹H NMR (300 MHz, DMSO-d₆) δ 7.84 (d, J=3.2 Hz, 1H), 7.74 (d, J=3.2 Hz, 1H), 7.43 (dd, J=9.4, 2.9 Hz, 1H), 7.19-7.10 (m Hz, 1H), 7.02 (dd, J=8.5, 5.6 Hz, 1H), 4.89 (s, 2H), 4.59 (s, 2H).

### Preparation 11.1

### Preparation of 3-(chloromethyl)-2-(2-(chloromethyl)-4-fluorophenylthio)thiophene

Following the procedure as described in Preparation 11, making non-critical variations to use (5-fluoro-2-(3-(hydroxymethyl)thiophen-2-ylthio)phenyl)methanol to replace (5-fluoro-2-(4-(hydroxymethyl)thiophen-3-ylthio)phenyl)methanol, 3-(chloromethyl)-2-(2-(chloromethyl)-4-fluorophenylthio)thiophene was obtained as a greenish oil in 98% yield: ¹H NMR (300 MHz, DMSO-d₆) δ 7.43 (d, J=5.5 Hz, 1H), 7.19 (d, J=5.5 Hz, 1H), 7.15 (dd, J=8.7, 3.0 Hz, 1H), 7.06 (dd, J=8.7, 5.5 Hz, 1H), 6.96-6.88 (m, 1H), 4.78 (s, 2H), 4.67 (s, 2H).

### Preparation 11.2

### Preparation of (2-(chloromethyl)-4-fluorophenyl)(2-(chloromethyl)-5-(methylsulfonyl)phenyl)sulfane

Following the procedure as described in Preparation 11, making non-critical variations to use (2-(4-fluoro-2-hydroxymethylphenylthio)-5-methanesulfonylphenyl)methanol to replace (5-fluoro-2-(4-(hydroxymethyl)thiophen-3-ylthio)phenyl)methanol, (2-(chloromethyl)-4-fluorophenyl)(2-(chloromethyl)-5-(methylsulfonyl)phenyl)sulfane was obtained as a pink solid in 84% yield: ¹H NMR (300 MHz, DMSO-d₆) δ 7.83-7.76 (m, 2H), 7.59 (dd, J=9.5, 2.9 Hz, 1H), 7.47 (dd, J=8.7, 5.7 Hz, 1H), 7.35-7.33 (m, 1H), 7.33-7.26 (m, 1H), 4.97 (s, 2H), 4.86 (s, 2H), 3.12 (s, 3H).

### Preparation 12

### Preparation of 4-chloro-2-(4-fluoro-2-methylphenoxy)-1-methylbenzene

A mixture of cuprous iodide (0.19 g, 1.00 mmol), N,N-dimethylglycine hydrochloride (0.42 g, 3.00 mmol), cesium carbonate (3.25 g, 10.00 mmol), 5-chloro-2-methylphenol (1.07 g, 7.50 mmol) and 1-bromo-4-fluoro-2-methylbenzene (1.32 g, 5.00 mmol) in dioxane (10 mL) was heated at 120° C. in a sealed tube for 16 h, cooled to ambient temperature, diluted with ethyl acetate (200 mL), washed with aqueous saturated sodium bicarbonate (2×20 mL), dried over anhydrous sodium sulfate and filtered. The filtrate was concentrated in vacuo and the residue was purified by column chromatography eluted with hexane to afford 4-chloro-2-(4-fluoro-2-methylphenoxy)-1-methylbenzene as a light yellow solid in 39% yield (0.42 g): ¹H NMR (300 MHz, CDCl₃) δ 7.89 (dd, J=8.3, 2.2 Hz, 1H), 7.46 (d, J=2.2 Hz, 1H), 7.39 (d, J=8.3 Hz, 1H), 7.21 (d, J=8.1 Hz, 1H), 7.09 (dd, J=8.1, 2.0 Hz, 1H), 6.77 (d, J=2.0 Hz, 1H), 2.39 (s, 3H), 2.19 (s, 3H).

### Preparation 12.1

### Preparation of 4-chloro-1-methyl-2-(2-methyl-5-nitrophenoxy)benzene

Following the procedure as described in Preparation 12, making non-critical variations using 2-iodo-4-nitrotoluene to replace 1-bromo-4-fluoro-2-methylbenzene to react with 5-chloro2-methylphenol, 4-chloro-1-methyl-2-(2-methyl-5-nitrophenoxy)benzene was obtained as a colorless solid in 30% yield: ¹H NMR (300 MHz, CDCl₃) δ 7.89 (dd, J=8.3, 2.2 Hz, 1H), 7.46 (d, J=2.2, Hz, 1H), 7.39 (d, J=8.3 Hz, 1H), 7.21 (d, J=8.1 Hz, 1H), 7.09 (dd, J=8.1, 2.0 Hz, 1H), 6.77 (d, J=1.9 Hz, 1H), 2.39 (s, 3H), 2.19 (s, 3H).

### Preparation 12.2

### Preparation of 1-chloro-3-(4-fluoro-2-methylphenoxy)-2-methylbenzene

Following the procedure as described in Preparation 12, making non-critical variations using 2-chloro-6-iodotoluene to replace 1-bromo-4-fluoro-2-methylbenzene to react with 4-fluoro-2-hydroxytoluene, 1-chloro-3-(4-fluoro-2-methylphenoxy)-2-methylbenzene was obtained as a colorless solid in 75% yield: ¹H NMR (300 MHz, CDCl₃) δ 7.08 (d, J=8.0 Hz, 1H), 7.02-6.92 (m, 2H), 6.83 (ddd, J=8.7, 8.7, 3.1 Hz, 1H), 6.72 (dd, J=8.9, 4.9 Hz, 1H), 6.50 (br d, J=8.1 Hz, 1H), 2.35 (s, 3H), 2.19 (s, 3H).

### Preparation 12.3

### Preparation of 4-chloro-1-(4-fluoro-2-methylphenoxy)-2-methylbenzene

Following the procedure as described in Preparation 12, making non-critical variations using 2-chloro-5-iodotoluene to replace 1-bromo-4-fluoro-2-methylbenzene to react with 4-fluoro-2-methylphenol, 4-chloro-1-(4-fluoro-2-methylphenoxy)-2-methylbenzene was obtained as a colorless solid in 52% yield: ¹H NMR (300 MHz, CDCl₃) δ 7.19 (d, J=2.6 Hz, 1H), 7.02 (dd, J=8.7, 2.6 Hz, 1H), 6.94 (dd, J=8.9, 3.0 Hz, 1H), 6.81 (ddd, J=8.7, 8.7, 3.0 Hz, 1H), 6.70 (dd, J=8.9, 4.9 Hz, 1H), 6.51 (d, J=8.7 Hz, 1H), 2.26 (s, 3H), 2.19 (s, 3H).

### Preparation 12.4

### Preparation of 4-fluoro-2-(4-fluoro-2-methylphenoxy)-1-methylbenzene

Following the procedure as described in Preparation 12, making non-critical variations using 2-bromo-4-fluorotoluene to replace 1-bromo-4-fluoro-2-methylbenzene to react with 4-fluoro-2-methylphenol, 4-fluoro-2-(4-fluoro-2-methylphenoxy)-1-methylbenzene was obtained as a colorless solid in 66% yield: ¹H NMR (300 MHz, CDCl₃) δ 7.14 (dd, J=7.7, 7.7 Hz, 1H), 6.96 (dd, J=9.0, 2.8 Hz, 1H), 6.90-6.76 (m, 2H), 6.65 (ddd, J=8.2, 8.2, 2.5 Hz, 1H), 6.26 (dd, J=10.2, 2.5 Hz, 1H), 2.26 (s, 3H), 2.18 (s, 3H).

### Preparation 12.5

### Preparation of 4,4′-oxybis(1-fluoro-3-methylbenzene)

Following the procedure as described in Preparation 12, making non-critical variations using 4-fluoro-2-methylphenol to replace 5-chloro-2-methylphenol to react with 1-bromo-4-fluoro-2-methylbenzene, 4,4′-oxybis(1-fluoro-3-methylbenzene) was obtained as a colorless solid in 69% yield: ¹H NMR (300 MHz, CDCl₃) δ 6.93 (dd, J=8.9, 3.0 Hz, 1H), 6.78 (ddd, J=8.5, 8.5, 3.1 Hz, 1H), 6.61 (dd, J=8.9, 4.9 Hz, 1H), 2.24 (s, 3H).

### Preparation 12.6

### Preparation of 4-fluoro-2-methyl-1-(2-methyl-4-nitrophenoxy)benzene

Following the procedure as described in Preparation 12, making non-critical variations using 2-iodo-5-nitrotoluene to replace 1-bromo-4-fluoro-2-methylbenzene to react with 4-fluoro-2-methylphenol, 4-fluoro-2-methyl-1-(2-methyl-4-nitrophenoxy)benzene was obtained as a light yellow solid in 88% yield: ¹H NMR (300 MHz, CDCl₃) δ 8.14 (d, J=2.7 Hz, 1H), 7.95 (dd, J=9.0, 2.7 Hz, 1H), 7.05-6.91 (m, 3H), 6.51 (d, J=9.0 Hz, 1H), 2.45 (s, 3H), 2.14 (s, 3H).

### Preparation 12.7

### Preparation of 4-chloro-2-methyl-1-(o-tolyloxy)benzene

Following the procedure as described in Preparation 12, making non-critical variations using 2-iodotoluene to replace 1-bromo-4-fluoro-2-methylbenzene to react with 4-chloro-2-methylphenol, 4-chloro-2-methyl-1-(o-tolyloxy)benzene was obtained as a colorless solid in 89% yield: ¹H NMR (300 MHz, CDCl₃) δ 7.27-7.00 (m, 5H), 6.73 (dd, J=7.8, 0.9 Hz, 1H), 6.62 (d, J=8.7 Hz, 1H), 2.27 (s, 3H), 2.26 (s, 3H).

### Preparation 12.8

### Preparation of 1-chloro-2-methyl-3-(o-tolyloxy)benzene

Following the procedure as described in Preparation 12, making non-critical variations using 2-iodotoluene to replace 1-bromo-4-fluoro-2-methylbenzene to react with 3-chloro-2-methylphenol, 1-chloro-2-methyl-3-(o-tolyloxy)benzene was obtained as a colorless solid in 86% yield: ¹H NMR (300 MHz, CDCl₃) δ 7.30-6.98 (m, 5H), 6.74 (d, J=8.1 Hz, 1H), 6.60 (d, J=8.1 Hz, 1H), 2.36 (s, 3H), 2.26 (s, 3H).

### Preparation 12.9

### Preparation of 4-chloro-1-methyl-2-(o-tolyloxy)benzene

Following the procedure as described in Preparation 12, making non-critical variations using 2-iodotoluene to replace 1-bromo-4-fluoro-2-methylbenzene to react with 5-chloro-2-methylphenol, 4-chloro-1-methyl-1-(o-tolyloxy)benzene was obtained as a colorless solid in 75% yield: ¹H NMR (300 MHz, CDCl₃) δ 7.31-7.04 (m, 4H), 6.97 (dd, J=8.1, 2.1 Hz, 1H), 6.82 (d, J=7.8 Hz, 1H), 6.65 (d, J=2.1 Hz, 1H), 2.29 (s, 3H), 2.26 (s, 3H).

### Preparation 12.10

### Preparation of 4-chloro-1-methyl-2-(2-methyl-4-nitrophenoxy)benzene

Following the procedure as described in Preparation 12, making non-critical variations using 2-iodo-5-nitrotoluene to replace 1-bromo-4-fluoro-2-methylbenzene to react with 5-chloro-2-methylphenol, 4-chloro-1-methyl-2-(2-methyl-4-nitrophenoxy)benzene was obtained as a colorless solid in 58% yield: ¹H NMR (300 MHz, CDCl₃) δ 8.19-8.14 (m, 1H), 7.99 (dd, J=9.0, 2.7 Hz, 1H), 7.28-7.11 (m, 2H), 6.92 (d, J=1.8 Hz, 1H), 6.62 (d, J=9.0 Hz, 1H), 2.43 (s, 3H), 2.16 (s, 3H).

### Preparation 12.11

### Preparation of 4-chloro-2-methyl-1-(2-methyl-4-nitrophenoxy)benzene

Following the procedure as described in Preparation 12, making non-critical variations using 2-iodo-5-nitrotoluene to replace 1-bromo-4-fluoro-2-methylbenzene to react with 4-chloro-2-methylphenol, 4-chloro-2-methyl-1-(2-methyl-4-nitrophenoxy)benzene was obtained as a colorless solid in 43% yield: ¹H NMR (300 MHz, CDCl₃) δ 8.17-8.13 (m, 1H), 7.96 (dd, J=9.0, 2.4 Hz, 1H), 7.32-7.18 (m, 2H), 6.88 (d, J=8.4 Hz, 1H), 6.56 (d, J=9.0 Hz, 1H), 2.44 (s, 3H), 2.16 (s, 3H).

### Preparation 12.12

### Preparation of 4-fluoro-2-methyl-1-(o-tolyloxy)benzene

Following the procedure as described in Preparation 12, making non-critical variations using 2-iodotoluene to replace 1-bromo-4-fluoro-2-methylbenzene to react with 4-fluoro-2-methylphenol, 4-fluoro-2-methyl-1-(o-tolyloxy)benzene was obtained as a colorless solid in 77% yield: ¹H NMR (300 MHz, CDCl₃) δ 7.29-6.62 (m, 7H), 2.31 (s, 3H), 2.25 (s, 3H).

### Preparation 13

### Preparation of 3-(bromomethyl)-4-(2-(bromomethyl)-4-chlorophenylthio)benzonitrile

To a stirred suspension of 4-(4-chloro-2-methylphenylthio)-3-methylbenzonitrile (1.20 g, 4.38 mmol) in carbon tetrachloride (35 mL) was added freshly re-crystallized N-bromosuccinimide (1.59 g, 8.80 mmol) followed by benzoyl peroxide (0.10 g, 0.44 mmol). The mixture was stirred at reflux for 12 h, diluted with dichloromethane (70 mL) and washed with water. The organic layer was separated, dried over sodium sulfate and filtered. The filtrate was concentrated in vacuo and the residue was triturated with ethyl acetate to afford 3-(bromomethyl)-4-(2-(bromomethyl)-4-chlorophenylthio)-benzonitrile as a colorless solid in 20% yield (0.385 g): ¹H NMR (300 MHz, DMSO-d₆) δ 8.03 (d, J=1.8 Hz, 1H), 7.81 (d, J=2.3 Hz, 1H), 7.64 (dd, J=1.8, 8.3 Hz, 1H), 7.50-7.38 (m, 2H), 6.91 (d, J=8.3 Hz, 1H), 4.83 (s, 2H), 4.74 (s, 2H).

### Preparation 13.1

### Preparation of 1-(bromomethyl)-2-(2-(bromomethyl)-4-fluorophenoxy)-4-chlorobenzene

Following the procedure as described in Preparation 13, making non-critical variations using 4-chloro-2-(4-fluoro-2-methylphenoxy)-1-methylbenzene to replace 4-(4-chloro-2-methylphenylthio)-3-methylbenzonitrile to react with N-bromosuccinimide, 1-(bromomethyl)-2-(2-(bromomethyl)-4-fluorophenoxy)-4-chlorobenzene was obtained as a white solid in 26% yield: ¹H NMR (300 MHz, CDCl₃) δ 7.19 (dd, J=8.5, 3.0 Hz, 1H), 7.16-7.13 (m, 2H), 7.05-6.96 (m, 1H), 6.88 (dd, J=8.0, 3.7 Hz, 1H), 6.68-6.60 (m, 1H), 4.79 (s, 2H), 4.53 (s, 2H).

### Preparation 13.2

### Preparation of 1-(bromomethyl)-2-(2-(bromomethyl)-5-chlorophenoxy)-4-nitrobenzene

Following the procedure as described in Preparation 13, making non-critical variations using 4-chloro-1-methyl-2-(2-methyl-5-nitrophenoxy)benzene to replace 4-(4-chloro-2-methylphenylthio)-3-methylbenzonitrile to react with N-bromosuccinimide, 1-(bromomethyl)-2-(2-(bromomethyl)-5-chlorophenoxy)-4-nitrobenzene was obtained as a white solid in 12% yield. R_{f}=0.25 (hexanes).

### Preparation 13.3

### Preparation of 2-(bromomethyl)-1-(2-(bromomethyl)-4-fluorophenoxy)-3-chlorobenzene

Following the procedure as described in Preparation 13, making non-critical variations using 1-chloro-3-(4-fluoro-2-methylphenoxy)-2-methylbenzene to replace 4-(4-chloro-2-methylphenylthio)-3-methylbenzonitrile to react with N-bromosuccinimide, 2-(bromomethyl)-1-(2-(bromomethyl)-4-fluorophenoxy)-3-chlorobenzene was obtained as a white solid in 50% yield: ¹H NMR (300 MHz, CDCl₃) δ 7.25-7.13 (m, 3H), 6.99 (ddd, J=7.8, 7.8, 2.9 Hz, 1H), 6.87 (dd, J=8.9, 4.7 Hz, 1H), 6.66-6.58 (m, 1H), 4.79 (s, 2H), 4.51 (s, 2H).

### Preparation 13.4

### Preparation of 2-(bromomethyl)-1-(2-(bromomethyl)-4-chlorophenoxy)-4-fluorobenzene

Following the procedure as described in Preparation 13, making non-critical variations using 4-chloro-1-(4-fluoro-2-methylphenoxy)-2-methylbenzene to replace 4-(4-chloro-2-methylphenylthio)-3-methylbenzonitrile to react with N-bromosuccinimide, 2-(bromomethyl)-1-(2-(bromomethyl)-4-chlorophenoxy)-4-fluorobenzene was obtained as a white solid in 37% yield: ¹H NMR (300 MHz, CDCl₃) δ 7.42 (d, J=2.2 Hz, 1H), 7.22-7.15 (m, 2H), 6.98 (ddd, J=8.9, 8.9, 3.0 Hz, 1H), 6.88 (dd, J=9.0, 4.6 Hz, 1H), 6.68 (d, J=8.7 Hz, 1H), 4.56 (s, 2H), 4.51 (s, 2H).

### Preparation 13.5

### Preparation of 1-(bromomethyl)-2-(2-(bromomethyl)-4-fluorophenoxy)-4-fluorobenzene

Following the procedure as described in Preparation 13, making non-critical variations using 4-fluoro-2-(4-fluoro-2-methylphenoxy)-1-methylbenzene to replace 4-(4-chloro-2-methylphenylthio)-3-methylbenzonitrile to react with N-bromosuccinimide, 1-(bromomethyl)-2-(2-(bromomethyl)-4-fluorophenoxy)-4-fluorobenzene was obtained as a white solid in 28% yield: ¹H NMR (300 MHz, CDCl₃) δ 7.40 (dd, J=8.5, 6.3 Hz, 1H), 7.21 (dd, J=8.5, 3.0 Hz, 1H), 7.02 (m, 1H), 6.92 (dd, J=9.0, 4.7 Hz, 1H), 6.78 (ddd, J=8.2, 8.2, 2.5 Hz, 1H), 6.41 (dd, J=9.9, 2.5 Hz, 1H), 4.62 (s, 2H), 4.0 (s, 2H).

### Preparation 13.6

### Preparation of 4,4′-oxybis(3-(bromomethyl)-1-fluorobenzene)

Following the procedure as described in Preparation 13, making non-critical variations using 4,4′-oxybis(1-fluoro-3-methylbenzene) to replace 4-(4-chloro-2-methylphenylthio)-3-methylbenzonitrile to react with N-bromosuccinimide, 4,4′-oxybis(3-(bromomethyl)-1-fluorobenzene) was obtained as a white solid in 10% yield: ¹H NMR (300 MHz, CDCl₃) δ 7.17 (dd, J=8.4, 3.1 Hz, 2H), 6.99-6.91 (m, 2H), 6.75 (dd, J=9.0, 4.6 Hz, 2H), 4.55 (s, 4H).

### Preparation 13.7

### Preparation of 2-(bromomethyl)-1-(2-(bromomethyl)-4-fluorophenoxy)-4-nitrobenzene

Following the procedure as described in Preparation 13, making non-critical variations using 4-fluoro-2-methyl-1-(2-methyl-4-nitrophenoxy)benzene to replace 4-(4-chloro-2-methylphenylthio)-3-methylbenzonitrile to react with N-bromosuccinimide, 2-(bromomethyl)-1-(2-(bromomethyl)-4-fluorophenoxy)-4-nitrobenzene was obtained as a colorless solid in 38% yield: ¹H NMR (300 MHz, CDCl₃) δ 8.36 (d, J=2.7 Hz, 1H), 8.16 (dd, J=9.0, 2.7 Hz, 1H), 7.30-7.23 (m, 1H), 7.10 (ddd, J=7.8, 7.8, 2.7 Hz, 1H), 7.01 (dd, J=9.0, 4.5 Hz, 1H), 6.75 (d, J=9.0 Hz, 1H), 4.69 (s, 2H), 4.48 (s, 2H).

### Preparation 13.8

### Preparation of 2,2′-oxybis((bromomethyl)benzene)

Following the procedure as described in Preparation 13, making non-critical variations using 2,2′-oxybis(methylbenzene) to replace 4-(4-chloro-2-methylphenylthio)-3-methylbenzonitrile to react with N-bromosuccinimide, 2,2′-oxybis((bromomethyl)benzene) was obtained as a colorless solid in 23% yield: ¹H NMR (300 MHz, CDCl₃) δ 7.47 (dd, J=7.5, 1.5 Hz, 2H), 7.27 (ddd, J=7.8, 7.8, 1.8 Hz, 2H), 7.12 (ddd, J=7.5, 7.5, 0.9 Hz, 2H), 6.84 (d, J=8.1 Hz, 2H), 4.65 (s, 4H).

### Preparation 13.9

### Preparation of 2-(bromomethyl)-1-(2-(bromomethyl)phenoxy)-4-chlorobenzene

Following the procedure as described in Preparation 13, making non-critical variations using 4-chloro-2-methyl-1-(o-tolyloxy)benzene to replace 4-(4-chloro-2-methylphenylthio)-3-methylbenzonitrile to react with N-bromosuccinimide, 2-(bromomethyl)-1-(2-(bromomethyl)phenoxy)-4-chlorobenzene was obtained as a colorless solid in 28% yield: ¹H NMR (300 MHz, CDCl₃) δ 7.51-7.42 (m, 2H), 7.33-7.09 (m, 3H), 6.83 (d, J=8.1 Hz, 1H), 6.76 (d, J=8.7 Hz, 1H), 4.61 (s, 2H), 4.57 (s, 2H).

### Preparation 13.10

### Preparation of 2-(bromomethyl)-1-(2-(bromomethyl)phenoxy)-3-chlorobenzene

Following the procedure as described in Preparation 13, making non-critical variations using 1-chloro-2-methyl-3-(o-tolyloxy)benzene to replace 4-(4-chloro-2-methylphenylthio)-3-methylbenzonitrile to react with N-bromosuccinimide, 2-(bromomethyl)-1-(2-(bromomethyl)phenoxy)-3-chlorobenzene was obtained as a colorless solid in 25% yield: ¹H NMR (300 MHz, CDCl₃) δ 7.49 (dd, J=7.5, 1.8 Hz, 1H), 7.34-7.12 (m, 4H), 6.89 (d, J=8.1 Hz, 1H), 6.72 (dd, J=7.5, 7.5 Hz, 1H), 4.81 (s, 2H), 4.61 (s, 2H).

### Preparation 13.11

### Preparation of 1-(bromomethyl)-2-(2-(bromomethyl)phenoxy)-4-chlorobenzene

Following the procedure as described in Preparation 13, making non-critical variations using 4-chloro-1-methyl-2-(o-tolyloxy)benzene to replace 4-(4-chloro-2-methylphenylthio)-3-methylbenzonitrile to react with N-bromosuccinimide, 1-(bromomethyl)-2-(2-(bromomethyl)phenoxy)-4-chlorobenzene was obtained as a colorless solid in 24% yield: ¹H NMR (300 MHz, CDCl₃) δ 7.49 (dd, J=7.5, 1.8 Hz, 1H), 7.39 (d, J=8.1 Hz, 1H), 7.32 (ddd, J=7.5, 7.5, 1.8 Hz, 1H), 7.18 (ddd, J=7.5, 7.5, 1.2 Hz, 1H), 7.08 (dd, J=8.1, 2.1 Hz, 1H), 6.90 (d, J=8.1 Hz, 1H), 6.78 (d, J=1.8 Hz, 1H), 4.61 (s, 2H), 4.59 (s, 2H).

### Preparation 13.12

### Preparation of 1-(bromomethyl)-2-(2-(bromomethyl)-4-nitrophenoxy)-4-chlorobenzene

Following the procedure as described in Preparation 13, making non-critical variations using 4-chloro-1-methyl-2-(2-methyl-4-nitrophenoxy)benzene to replace 4-(4-chloro-2-methylphenylthio)-3-methylbenzonitrile to react with N-bromosuccinimide, 1-(bromomethyl)-2-(2-(bromomethyl)-4-nitrophenoxy)-4-chlorobenzene was obtained as a colorless solid in 19% yield: ¹H NMR (300 MHz, CDCl₃) δ 8.38 (d, J=2.7 Hz, 1H), 8.16 (d, J=9.0, 2.7 Hz, 1H), 7.47 (d, J=8.4 Hz, 1H), 7.25 (dd, J=8.1, 2.1 Hz, 1H), 6.99 (d, J=2.1 Hz, 1H), 6.87 (d, J=9.0 Hz, 1H), 4.67 (s, 2H), 4.52 (s, 2H).

### Preparation 13.13

### Preparation of 2-(bromomethyl)-1-(2-(bromomethyl)-4-chlorophenoxy)-4-nitrobenzene

Following the procedure as described in Preparation 13, making non-critical variations using 4-chloro-2-methyl-1-(2-methyl-4-nitrophenoxy)benzene to replace 4-(4-chloro-2-methylphenylthio)-3-methylbenzonitrile to react with N-bromosuccinimide, 1-(bromomethyl)-2-(2-(bromomethyl)-4-nitrophenoxy)-4-chlorobenzene was obtained as a colorless solid in 35% yield: ¹H NMR (300 MHz, CDCl₃) δ 8.37 (d, J=2.1 Hz, 1H), 8.17-8.08 (m, 1H), 7.53 (d, J=1.8 Hz, 1H), 7.35 (dd, J=8.7, 1.8 Hz, 1H), 6.95 (d, J=8.7 Hz, 1H), 6.80 (d, J=9.0 Hz, 1H), 4.68 (s, 2H), 4.49 (s, 2H).

### Preparation 13.14

### Preparation of 2-(bromomethyl)-1-(2-(bromomethyl)phenoxy)-4-fluorobenzene

Following the procedure as described in Preparation 13, making non-critical variations using 4-fluoro-2-methyl-1-(o-tolyloxy)benzene to replace 4-(4-chloro-2-methylphenylthio)-3-methylbenzonitrile to react with N-bromosuccinimide, 2-(bromomethyl)-1-(2-(bromomethyl)phenoxy)-4-fluorobenzene was obtained as a colorless solid in 37% yield: ¹H NMR (300 MHz, CDCl₃) δ 7.46 (dd, J=7.5, 1.5 Hz, 1H), 7.29-6.82 (m, 5H), 6.76 (d, J=8.1 Hz, 1H), 4.65 (s, 2H), 4.56 (s, 2H).

### Preparation 13.15

### Preparation of bis(2-(bromomethyl)phenyl)sulfane

Following the procedure as described in Preparation 13, making non-critical variations using di-o-tolylsulfane to replace 4-(4-chloro-2-methylphenylthio)-3-methylbenzonitrile to react with N-bromosuccinimide, bis(2-(bromomethyl)phenyl)sulfane was obtained as a colorless solid in 97% yield: ¹H NMR (300 MHz, CDCl₃) δ 7.45 (d, J=7.3 Hz, 2H), 7.29-7.11 (m, 6H), 4.69 (s, 4H).

### Preparation 14

### Preparation of 1-(hydroxymethyl)-2-((6-(hydroxymethyl)phenyl)thio)benzene

### A. Preparation of 2,2′-thiodibenzoic acid

Sodium hydroxide (40.00 g, 1000.00 mmol) was added to a solution of 2-(2-cyanophenylthio)benzoic acid (72.00 g, 282.35 mmol) in water (600 mL). The mixture was refluxed for 16 h, cooled to ambient temperature and filtered. The resulting solid was washed with ether (2×100 mL) and dried in high vacuum at 80° C. to afford 2,2′-thiodibenzoic acid as a white solid in 97% yield (74.88 g): ¹H NMR (300 MHz, DMSO-d₆) δ 13.12 (s, 2H), 7.80 (dd, J=7.6, 1.5 Hz, 2H), 7.46-7.38 (m, 2H), 7.38-7.30 (m, 2H), 7.07 (dd, J=7.8, 0.8 Hz, 2H); MS (ES+) m/z 273.2 (M−1).

### B. Preparation of 1-(hydroxymethyl)-2-((6-(hydroxymethyl)phenyl)thio)benzene

To an ice-cold solution of 2,2′-thiodibenzoic acid (50.00 g, 182.46 mmol) in tetrahydrofuran (130 mL) was added dropwise borane in tetrahydrofuran (570 mL of 1 M solution, 570.70 mmol). The temperature was then raised to ambient temperature and stirred under nitrogen for 16 h. The reaction mixture was diluted slowly with methanol (100 mL) followed by the addition of water (400 mL). A white solid was precipitated upon basification of the reaction mixture with aqueous saturated sodium bicarbonate solution. Methanol was removed in vacuo and the residue was filtered. The resulting solid was dried in high vacuum at 60° C. to afford 1-(hydroxymethyl)-2-((6-(hydroxymethyl)phenyl)thio)benzene as white solid in 95% yield (42.46 g): ¹H NMR (300 MHz, DMSO-d₆) δ 7.49-7.43 (m, 2H), 7.32-7.09 (m, 6H), 4.74 (s, 4H), 2.15 (s, 2H); MS (ES+) m/z 230 (M−17).

### Preparation 15

### Preparation of 1-(2-aminoethyl)-2-((6-(2-aminoethyl)phenyl)thio)benzene dihydrochloride

### A. Preparation of bis(2-(bromomethyl)phenyl)sulfane

Phosphorus tribromide (31.43 mL, 333.24 mmol) was added drop wise to a solution of 1-(hydroxymethyl)-2-((6-(hydroxymethyl)phenyl)thio)benzene (41.00 g, 166.62 mmol) in ether (1000 mL). The mixture was stirred at ambient temperature for 3 h under nitrogen, followed by the addition of crushed ice (300 g). The organic layer was separated and filtered through a small plug of silica gel and concentrated in vacuo. The residue was recrystallized from ether to afford bis(2-(bromomethyl)phenyl)sulfane as a white solid in 77% yield (47.50 g): ¹H NMR (300 MHz, CDCl₃) δ 7.46 (dd, J=7.4, 1.6 Hz, 2H), 7.29-7.12 (m, 6H) 4.69 (s, 4H).

### B. Synthesis of 1-(cyanomethyl)-2-((6-(cyanomethyl)phenyl)thio)benzene dihydrochloride

A mixture of bis(2-(bromomethyl)phenyl)sulfane (31.90 g, 86.24 mmol) and sodium cyanide (8.88 g, 181.10 mmol) in water (5 mL) and ethanol (150 mL) was refluxed for 16 h under nitrogen. The reaction mixture was concentrated and the residue was dissolved in ethyl acetate (1000 mL). The organic layer was dried over sodium sulfate, filtered and concentrated in vacuo. The residue was recrystallized from ethyl acetate/hexane to afford 1-(cyanomethyl)-2-((6-(cyanomethyl)phenyl)thio)benzene as a light yellow solid in 88% yield (20.00 g): ¹H NMR (300 MHz, CDCl₃) δ 7.56 (d, J=7.6 Hz, 2H), 7.36 (dd, J=7.6, 7.6 Hz, 2H), 7.28 (d, J=7.7 Hz, 2H), 7.11 (d, J=7.7 Hz, 2H), 3.84 (s, 4H); MS (ES+) m/z 287.2 (M+23).

### C. Preparation of 1-(2-aminoethyl)-2-((6-(2-aminoethyl)phenyl)thio)benzene dihydrochloride

To an ice-cold solution of 1-(cyanomethyl)-2-((6-(cyanomethyl)phenyl)thio)benzene (12.00 g, 45.33 mmol) in tetrahydrofuran (500 mL) was added dropwise borane in tetrahydrofuran (120.00 mL of 1 M solution, 120.00 mmol). The reaction mixture was heated at reflux under nitrogen for 16 h, cooled to ambient temperature and quenched slowly with water (110 mL). The pH of the solution was adjusted to ˜2 with hydrochloric acid (37%) slowly and a white solid was obtained upon acidification. The resulting mixture was stirred for 16 h and diluted with ether (500 mL) and filtered. The solid was collected and dried in high vacuum at 60° C. to afford 1-(2-aminoethyl)-2-((6-(2-aminoethyl)phenyl)thio)benzene dihydrochloride as a white solid in 51% yield (8.00 g): ¹H NMR (300 MHz, DMSO-d₆) δ 8.45-8.16 (br s, 6H), 7.45-7.21 (m, 6H), 7.07-7.01 (m, 2H), 3.15-2.95 (m, 8H); ¹³C NMR (75 MHz, DMSO-d₆) δ 137.7, 133.2, 131.2, 129.8, 127.9, 30.7; MS (ES+) m/z 273.4 (M+1).

### Preparation 16

### Preparation of 1-(2-cyanoethyl)-2-((6-(2-cyanoethyl)phenyl)thio)benzene

To a stirred solution of 1-(hydroxymethyl)-2-((6-(hydroxymethyl)phenyl)thio)benzene (0.49 g, 2.00 mmol) and (cynomethyl)trimethylphosphonium iodide (2.48 g, 10.2 mmol, prepared according to F. Zaragoza et al., J. Org. Chem. 2001, 66, 2518-2521) in propionitrile (8.0 mL) was added diisopropylethylamine (2.2 mL, 12.6 mmol) slowly at ambient temperature. The mixture was stirred at 97° C. for 24 hours, followed by the addition of water (0.40 mL, 22.2 mmol). The mixture was stirred at 97° C. for another 15 hours followed by the addition of water (25 mL) and concentrated hydrochloric acid (2.0 mL). The mixture was extracted with ethyl acetate (3×30 mL). The combined organic layers was dried over anhydrous sodium sulfate and filtered. The filtrate was concentrated in vacuo. The residue was purified by column chromatography (ethyl acetate/hexane, 1/5) to afford 1-(2-cyanoethyl)-2-((6-(2-cyanoethyl)phenyl)thio)benzene as a colorless oil in 43% yield (0.25 g): ¹H NMR (300 MHz, CDCl₃) δ 7.36-7.23 (m, 4H), 7.20 (ddd, J=7.6, 7.6, 2.0 Hz, 2H), 7.05 (dd, J=7.6, 1.5 Hz, 2H), 3.10 (t, J=7.3 Hz, 4H), 2.65 (t, J=7.3 Hz, 4H); ¹³C NMR (75 MHz, CDCl₃) δ 138.5, 133.6, 132.1, 130.3, 128.7, 128.2, 118.9, 29.9, 18.1; MS (ES+) m/z 293.3 (M+1).

### Preparation 17

### Preparation of 1-(3-cyanopropyl)-2-((6-(3-cyanopropyl)phenyl)thio)benzene

A mixture of manganese and chromium(III) chloride was flushed with argon for 20 minutes before the addition of tetrahydrofuran (10 mL), 4-tert-butylpyridine (1.0 mL, 6.8 mmol) and water (3.8 μL, 0.27 mmol). The mixture was stirred for 3 hours at ambient temperature, followed by the addition of acrylonitrile (0.45 mL, 6.80 mmol) and bis(2-(bromomethyl)phenyl)sulfane (0.25 g, 0.68 mmol). The mixture was stirred at ambient temperature for 16 hours and saturated ammonium chloride solution (25 mL) was added. The mixture was extracted with ethyl acetate (3×30 mL). The combined organic layers was dried over anhydrous sodium sulfate and filtered. The filtrate was concentrated in vacuo. The residue was purified by column chromatography (ethyl acetate/hexane, 1/5) to afford 1-(3-cyanopropyl)-2-((6-(3-cyanopropyl)phenyl)thio)benzene as an colorless oil in 75% yield (0.16 g): ¹H NMR (300 MHz, CDCl₃) δ 7.28-7.10 (m, 6H), 7.03 (d, J=7.3 Hz, 2H), 2.91 (t, J=7.6 Hz, 4H), 2.35 (t, J=7.6 Hz, 4H), 2.05-1.92 (m, 4H); ¹³C NMR (75 MHz, CDCl₃) δ 140.5, 134.2, 132.1, 130.1, 127.8, 127.7, 119.5, 32.9, 26.0, 16.8; MS (ES+) m/z 321.3 (M+1).

### Preparation 18

### Preparation of Di-o-Tolylmethanone

### A. Preparastion of di-o-tolylmethanimine

To a solution of 2-methylbenzonitrile (13.50 mL, 115.00 mmol) in anhydrous tetrahydrofuran (100 mL) was added 2-methylphenylmagnesium bromide (55.2 mL, 166.0 mmol) dropwise over 15 minutes at 0° C. The resulting solution was heated at reflux under a nitrogen atmosphere for 16 hours then poured over ice (50 g). The mixture was extracted with ethyl acetate (3×20 mL) and combined organic layer was washed with brine (30 mL), dried over anhydrous sodium sulfate, filtered and concentrated in vacuo. The residue was purified by flash chromatography eluted with a gradient of 0 to 20% ethyl acetate in hexanes to afford di-o-tolylmethanimine as clear oil in 97% yield (23.10 g): ¹H NMR (300 MHz, CDCl₃) δ 9.77 (br s, 1H), 7.32-7.15 (m, 8H), 2.26 (s, 6H); ¹³C NMR (75 MHz, DMSO-d₆) δ 180.6, 131.1, 129.3, 125.9, 20.5; MS (ES+) m/z 210.3 (M+1).

### B. Preparation of di-o-tolylmethanone

A mixture of di-o-tolylmethanimine (19.2 g, 91.8 mmol) in 1-propanol (120 mL) and 6 N hydrochloric acid (80 mL) was stirred at ambient temperature for 16 h and neutralized with 1 M sodium hydroxide solution. The mixture was extracted with dichloromethane (3×30 mL) and the combined organic layers was washed with brine (30 mL), dried over anhydrous sodium carbonate, filtered and concentrated in vacuo. The residue was purified by flash chromatography eluted with a gradient of 0 to 20% ethyl acetate in hexanes to afford di-o-tolylmethanone as a beige solid in 82% yield (15.70 g): mp 66-67° C.; ¹H NMR (300 MHz, CDCl₃) δ 7.42-7.17 (m, 8H), 2.44 (s, 6H); ¹³C NMR (75 MHz, CDCl₃) 200.8, 139.0, 138.1, 131.4, 131.1, 130.3, 125.4, 20.7.

### Preparation 18.1

### Preparation of (4-fluoro-2-methylphenyl)(o-tolyl)methanone

Following the procedure as described in Preparation 18, making non-critical variations using 4-fluoro-2-methylphenylmagnesium bromide to replace 2-methylphenylmagnesium bromide to react with 2-methylbenzonitrile followed by the hydrolysis under acidic conditions, (4-fluoro-2-methylphenyl)(o-tolyl)methanone was obtained as a clear oil in 68% yield (over two steps): ¹H NMR (300 MHz, CDCl₃) δ 7.40-7.15 (m, 5H), 6.96 (dd, J=9.7, 2.4 Hz, 1H), 6.92-6.80 (m, 1H), 2.46 (s, 3H), 2.39 (s, 3H); ¹³C NMR (75 MHz, CDCl₃) δ 199.4, 167.7, 162.4, 142.1 (d, J_{C-F}=8.9 Hz), 139.1, 137.9, 135.0, 133.2 (d, J_{C-F}=9.3 Hz), 131.2 (d, J_{C-F}=28.5 Hz), 129.9, 125.5, 118.4 (d, J_{C-F}=21.3 Hz), 112.4, (d, J_{C-F}=21.4 Hz), 21.0, 20.5.

### Preparation 18.2

### Preparation of (5-fluoro-2-methylphenyl)(o-tolyl)methanone

Following the procedure as described in Preparation 18, making non-critical variations using 5-fluoro-2-methylphenylmagnesium bromide to replace 2-methylphenylmagnesium bromide to react with 2-methylbenzonitrile followed by the hydrolysis under acidic conditions, (5-fluoro-2-methylphenyl)(o-tolyl)methanone was obtained as a yellow oil in 91% yield (over two steps): ¹H NMR (300 MHz, CDCl₃) δ 7.46-7.38 (m, 1H), 7.37-7.31 (m, 2H), 7.26-7.23 (m, 2H), 7.15-7.06 (m, 1H), 7.05 (dd, J=8.9, 2.7 Hz, 1H), 2.52 (s, 3H), 2.40 (s, 3H); ¹³C NMR (75 MHz, CDCl₃) δ 199.3, 162.1, 158.8, 140.5 (d, J_{C-F}=5.6 Hz), 138.6, 137.9, 133.4 (d, J_{C-F}=3.5 Hz), 132.8 (d, J_{C-F}=7.2 Hz), 131.6 (d, J_{C-F}=7.2 Hz), 130.6, 125.6, 117.8 (d, J_{C-F}=20.9 Hz), 116.5 (d, J_{C-F}=22.4 Hz), 20.8, 19.8.

### Preparation 19

### Preparation of (5-chloro-2-methylphenyl)(o-tolyl)methanone

To a mixture of 5-chloro-2-methylphenylboronic acid (3.00 g, 17.60 mmol), cesium carbonate (11.50 g, 35.20 mmol), tetrakis(triphenylphosphene)palladium(0) (1.02 g, 0.85 mmol) in anhydrous toluene (60 mL) was added o-toluoyl chloride (4.60 mL, 35.20 mmol). The reaction mixture was heated at 80° C. for 16 hours, cooled to ambient temperature and filtered through a pad of celite. The pad was washed with ethyl acetate (50 mL) and the filtrate was concentrated in vacuo. The residue was purified by flash chromatography eluted with a gradient of 0 to 20% ethyl acetate in hexanes to afford (5-chloro-2-methylphenyl)(o-tolyl)methanone as a colorless oil in 75% yield (3.20 g). ¹H NMR (300 MHz, CDCl₃) δ 7.42-7.26 (m, 5H), 7.22-7.16 (m, 2H), 2.47 (s, 3H), 2.34 (s, 3H): ¹³C NMR (75 MHz, CDCl₃) δ 199.2, 140.7, 138.7, 137.8, 136.2, 132.7, 131.7, 131.7, 131.3, 130.8, 130.7, 129.5, 125.6, 20.9, 20.0.

### Preparation 19.1

### Preparation of (4-chloro-2-methylphenyl)(o-tolyl)methanone

Following the procedure as described in Preparation 19, making non-critical variations using 2-methylphenylboronic acid to replace 5-chloro-2-methylphenylboronic acid to react with 4-chloro-2-methylbenzoyl chloride, (4-chloro-2-methylphenyl)(o-tolyl)methanone was obtained as a clear oil in 75% yield: ¹H NMR (300 MHz, CDCl₃) δ 7.41-7.35 (m, 1H), 7.28-7.71 (m, 6H), 2.41 (s, 6H).

### Preparation 20

### Preparation of bis(2-(bromomethyl)phenyl)methanone

A suspension of di-o-tolylmethanone (1.00 g, 4.80 mmol), N-bromosuccinimide (1.69 g, 9.60 mmol) and benzoyl peroxide (0.005 g) in carbon tetrachloride (60 mL) was heated at reflux for 3 hours under a nitrogen atmosphere. The reaction mixture was cooled to ambient temperature and filtered. The filtrate was concentrated in vacuo. The residue was purified by flash chromatography eluted with a gradient of 0 to 20% ethyl acetate in hexanes to afford bis(2-(bromomethyl)phenyl)methanone as a colorless solid (0.33 g) in 20% yield: mp 113-114° C.; ¹H NMR (300 MHz, CDCl₃) δ 7.33-7.27 (m, 1H), 7.25-7.17 (m, 5H), 6.96-6.83 (m, 2H), 2.28 (s, 3H), 2.22 (t, J=6.0 Hz, 3H); ¹³C NMR (75 MHz, CDCl₃) δ 199.4, 167.7, 162.4, 142.1 (d, J_{C-F}=8.9 Hz), 139.1, 137.9, 135.0, 133.2 (d, J_{C-F}=9.3 Hz), 131.2 (d, J_{C-F}=28.5 Hz), 129.9, 125.5, 118.4 (d, J_{C-F}=21.3 Hz), 112.4, (d, J_{C-F}=21.4 Hz), 21.0, 20.5.

### Preparation 20.1

### Preparation of (2-(bromomethyl)-4-fluorophenyl)(2-(bromomethyl)phenyl)methanone

Following the procedure as described in Preparation 20, making non-critical variations using (4-fluoro-2-methylphenyl)(o-tolyl)methanone to replace di-o-tolylmethanone to react with N-bromosuccinimide, (2-(bromomethyl)-4-fluorophenyl)(2-(bromomethyl)phenyl)methanone was obtained as a white solid in 17% yield: mp 98-99° C.; ¹H NMR (300 MHz, CDCl₃) δ 7.53-7.45 (m, 2H), 7.38-7.20 (m, 4H), 7.02-6.94 (m, 1H), 4.84 (s, 2H), 4.82 (s, 2H); ¹³C NMR (75 MHz, CDCl₃) δ 197.8, 165.9, 142.1 (d, J_{C-F}=8.4 Hz), 138.4 (d, J_{C-F}=8.3 Hz), 134.5 (d, J_{C-F}=9.2 Hz), 131.8, 131.4, 131.1, 128.1, 118.8 (d, J_{C-F}=21.5 Hz), 115.0, (d, J_{C-F}=21.5 Hz), 30.7, 30.0.

### Preparation 20.2

### Preparation of (2-(bromomethyl)-5-fluorophenyl)(2-(bromomethyl)phenyl)methanone

Following the procedure as described in Preparation 20, making non-critical variations using (5-fluoro-2-methylphenyl)(o-tolyl)methanone to replace di-o-tolylmethanone to react with N-bromosuccinimide, (2-(bromomethyl)-5-fluorophenyl)(2-(bromomethyl)phenyl)methanone was obtained as a clear oil in 10% yield: mp 118-119° C.; ¹H NMR (300 MHz, CDCl₃) δ 7.53-7.50 (m, 3H), 7.35-7.30 (m, 2H), 7.02-6.94 (m, 1H), 7.03 (dd, J=8.6, 2.6 Hz, 1H), 4.87 (s, 2H), 4.79 (s, 2H).

### Preparation 20.3

### Preparation of (2-(bromomethyl)-5-chlorophenyl)(2-(bromomethyl)phenyl)methanone

Following the procedure as described in Preparation 20, making non-critical variations using (5-chloro-2-methylphenyl)(o-tolyl)methanone to replace di-o-tolylmethanone to react with N-bromosuccinimide, (2-(bromomethyl)-5-chlorophenyl)(2-(bromomethyl)phenyl)methanone was obtained as a colorless solid in 35% yield: mp 152-153° C.; ¹H NMR (300 MHz, CDCl₃) δ 7.42-7.26 (m, 5H), 7.22-7.16 (m, 2H), 2.47 (s, 3H), 2.34 (s, 3H).

### Preparation 20.4

### Preparation of (2-(bromomethyl)-4-chlorophenyl)(2-(bromomethyl)phenyl)methanone

Following the procedure as described in Preparation 20, making non-critical variations using (4-chloro-2-methylphenyl)(o-tolyl)methanone to replace di-o-tolylmethanone to react with N-bromosuccinimide, (2-(bromomethyl)-4-chlorophenyl)(2-(bromomethyl)phenyl)methanone was obtained as a colorless solid in 20% yield: mp 152-153° C.; ¹H NMR (300 MHz, CDCl₃) δ 7.52-7.50 (m, 2H), 7.45-7.43 (m, 2H), 7.35-7.33 (m, 2H), 7.30-7.28 (m, 1H), 4.30 (s, 2H), 4.77 (s, 2H); ¹³C NMR (75 MHz, CDCl₃) δ 197.4, 139.8, 138.8, 137.0, 136.8, 134.1, 132.7, 132.4, 131.8, 131.7, 131.6, 130.9, 128.3, 30.6, 29.6.

### Preparation 21

### Preparation of 2-(2-((6-cyanomethylphenyl)thio)phenyl)acetonitrile

To a stirred solution of 1-(hydroxymethyl)-2-((6-(hydroxymethyl)phenyl)thio)benzene (3.55 g, 14.40 mmol) in chloroform (50 mL) was added thionylchloride (10.50 g, 144.00 mmol) all at once at ambient temperature. The mixture was stirred at ambient temperature for five hours and concentrated in vacuo. The residue was dissolved in ethyl acetate (150 mL) and washed with saturated sodium bicarbonate solution. The organic layer was dried over anhydrous sodium sulfate and filtered. The filtrate was concentrated in vacuo. The residue was dissolved in ethanol (30 mL). A solution of sodium cyanide (2.10 g, 43.20 mmol) in water (5 mL) was added to the ethanol solution with stirring at ambient temperature. The mixture was refluxed for 16 hours and concentrated in vacuo. The residue was dissolved in ethyl acetate (150 mL) and washed with water. The organic layer was dried over anhydrous sodium sulfate and filtered. The filtrate was concentrated in vacuo. The residue was purified by column chromatography eluted with ethyl acetate/hexane (1/5) to afford 2-(2-((6-cyanomethylphenyl)thio)phenyl)acetonitrile as a colorless oil in 54% yield (2.1 g): ¹H NMR (300 MHz, CDCl₃) δ 7.51-7.46 (m, 2H), 7.32-7.12 (m, 6H), 4.77 (s, 4H).

### Example 1

### Synthesis of 2-(1-{2-[2-(1-carbamimidoylsulfanylethyl)phenoxy]phenyl}ethyl)-isothiourea dihydrobromide

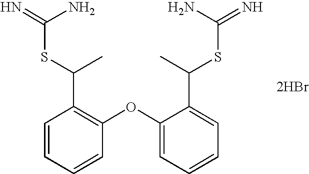

To a stirred suspension of thiourea (0.17 g, 0.22 mmol) in water was added 48% aqueous hydrobromic acid. The mixture was stirred for 10 min followed by the addition of 1-(1-hydroxyethyl)-2-(6-(1-hydroxyethyl)phenoxy)benzene (0.26 g, 0.10 mmol) in one portion. The mixture was stirred at 80° C. for 3 h and cooled to ambient temperature. Ether was added (2.0 mL) to the mixture. The solid was collected by filtration and dried in air to afford 2-(1-{2-[2-(1-carbamimidoylsulfanylethyl)phenoxy]phenyl}ethyl)isothiourea dihydrobromide in 54% yield (0.19 g): ¹H NMR (300 MHz, DMSO-d₆) δ 9.22 (s, 4H), 9.03 (s, 4H), 7.64 (d, J=8.8 Hz, 2H), 7.35 (t, J=7.0 Hz, 2H), 7.22 (t, J=7.5 Hz, 2H), 6.75 (d, J=8.2 Hz, 2H), 6.22 (s, 2H), 5.44 (q, J=6.8 Hz, 2H), 1.76 (d, J=6.8 Hz, 6H); MS (ES+) m/z 358.6 (M+1).

### Example 1.1

### Synthesis of 2-[2-(2-carbamimidoylsulfanylmethylphenylsulfanyl)benzyl]isothiourea dihydrobromide

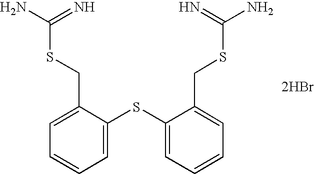

Following the procedure as described in Example 1, making non-critical variations using 1-(hydroxymethyl)-2-((6-(hydroxymethyl)phenyl)thio)benzene to replace 1-(1-hydroxyethyl)-2-(6-(1-hydroxyethyl)phenoxy)benzene, 2-[2-(2-carbamimidoylsulfanylmethylphenylsulfanyl)benzyl]isothiourea dihydrobromide was obtained as a colorless solid in 74% yield: ¹H NMR (300 MHz, DMSO-d₆) δ 9.28 (s, 4H), 9.10 (s, 4H), 7.63-7.59 (m, 2H), 7.41-7.32 (m, 4H), 7.15-7.10 (m, 2H), 4.65 (s, 4H); MS (ES+) m/z 363.6 (M+1).

### Example 1.2

### Synthesis of 2-[2-(2-carbamimidoylsulfanylmethyl-4-fluorophenylsulfanyl)benzyl]-isothiourea dihydrobromide

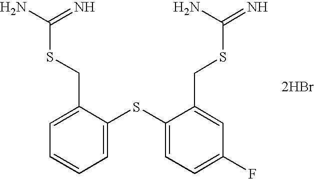

Following the procedure as described in Example 1, making non-critical variations using (5-fluoro-2-(2-(hydroxymethyl)phenylthio)phenyl)methanol to replace 1-(1-hydroxyethyl)-2-(6-(1-hydroxyethyl)phenoxy)benzene, 2-[2-(2-carbamimidoylsulfanylmethyl-4-fluorophenylsulfanyl)benzyl]-isothiourea dihydrobromide was obtained as a colorless solid in 69% yield: ¹H NMR (300 MHz, DMSO-d₆) δ 9.23 (s, 4H), 9.06 (s, 4H), 7.56-7.51 (m, 2H), 7.34-7.29 (m, 2H), 7.27-7.23 (m, 2H), 7.01-6.98 (m, 1H), 4.61 (s, 2H), 4.58 (s, 2H); MS (ES+) m/z 381.1 (M+1).

### Example 1.3

### Synthesis of 2-[2-(2-carbamimidoylsulfanylmethyl-5-methylphenylsulfanyl)-benzyl]isothiourea dihydrobromide

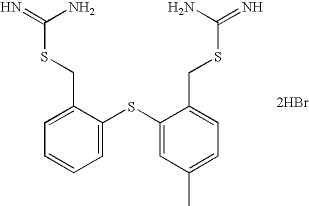

Following the procedure as described in Example 1, making non-critical variations using (2-(2-(hydroxymethyl)-5-methylphenylthio)phenyl)methanol to replace 1-(1-hydroxyethyl)-2-(6-(1-hydroxyethyl)phenoxy)benzene, 2-[2-(2-carbamimidoylsulfanylmethyl-5-methylphenylsulfanyl)-benzyl]isothiourea dihydrobromide was obtained as a colorless solid in 35% yield: ¹H NMR (300 MHz, DMSO-d₆) δ 9.24 (br s, 4H), 9.09 (br s, 4H), 7.57-7.51 (m, 1H), 7.47 (d, J=7.8 Hz, 1H), 7.35-7.25 (m, 2H), 7.18 (dd, J=1.0, 7.8 Hz, 1H), 7.05-7.01 (m, 1H), 6.97 (d, J=1.0 Hz, 1H), 4.61 (s, 2H), 4.55 (s, 2H), 2.18 (s, 3H); MS (ES+) m/z 377.1 (M+1).

### Example 1.4

### Synthesis of 2-[2-(2-carbamimidoylsulfanylmethyl-4-methoxyphenylsulfanyl)-benzyl]isothiourea dihydrobromide

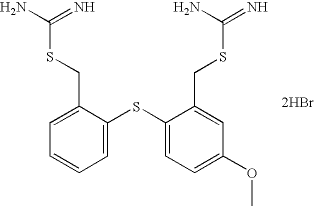

Following the procedure as described in Example 1, making non-critical variations using (2-(2-(hydroxymethyl)-4-methoxyphenylthio)phenyl)methanol to replace 1-(1-hydroxyethyl)-2-(6-(1-hydroxyethyl)phenoxy)benzene, 2-[2-(2-carbamimidoylsulfanylmethyl-4-methoxyphenylsulfanyl)-benzyl]isothiourea dihydrobromide was obtained as a colorless solid in 57% yield: ¹H NMR (300 MHz, DMSO-d₆) δ 9.23 (s, 2H), 9.19 (s, 2H), 9.06 (s, 2H), 9.02 (s, 2H), 7.45-7.53 (m, 1H), 7.32 (d, J=8.6 Hz, 1H), 7.26 (d, J=2.8 Hz, 1H), 7.18-7.24 (m, 2H), 6.99 (dd, J=2.8, 8.7 Hz, 1H), 6.80-6.72 (m, 1H), 4.61 (s, 2H), 4.51 (s, 2H), 3.77 (s, 3H); MS (ES+) m/z 393.1 (M+1).

### Example 1.5

### Synthesis of 2-[2-(2-carbamimidoylsulfanylmethyl-5-methylphenylsulfanyl)-5-fluorobenzyl]isothiourea dihydrobromide

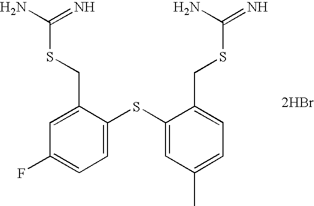

Following the procedure as described in Example 1, making non-critical variations using (5-fluoro-2-(2-(hydroxymethyl)-5-methylphenylthio)phenyl)methanol to replace 1-(1-hydroxyethyl)-2-(6-(1-hydroxyethyl)phenoxy)benzene, 2-[2-(2-carbamimidoylsulfanylmethyl-5-methylphenylsulfanyl)-5-fluorobenzyl]isothiourea dihydrobromide was obtained as a colorless solid in 32% yield: ¹H NMR (300 MHz, DMSO-d₆) δ 9.23 (s, 2H), 9.17 (s, 2H), 9.06 (s, 2H), 9.02 (s, 2H), 7.50 (dd, J=2.6, 9.5 Hz, 1H), 7.44 (d, J=7.8 Hz, 1H), 7.23 (m, 1H), 7.19 (d, J=5.7 Hz, 1H), 7.15 (m, 1H), 6.88 (d, J=0.7 Hz, 1H), 4.58 (s, 2H), 4.55 (s, 2H), 2.17 (s, 3H); MS (ES+) m/z 395.1 (M+1).

### Example 1.6

### Synthesis of 2-[2-(2-carbamimidoylsulfanylmethyl-4-methoxyphenylsulfanyl)-5-fluorobenzyl]isothiourea dihydrobromide

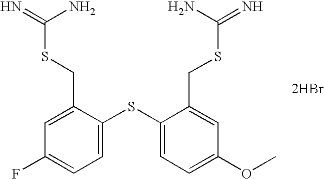

Following the procedure as described in Example 1, making non-critical variations using (5-fluoro-2-(2-(hydroxymethyl)-5-methoxyphenylthio)phenyl)methanol to replace 1-(1-hydroxyethyl)-2-(6-(1-hydroxyethyl)phenoxy)benzene, 2-[2-(2-carbamimidoylsulfanylmethyl-4-methoxyphenylsulfanyl)-5-fluorobenzyl]isothiourea dihydrobromide was obtained as a colorless solid in 91% yield: ¹H NMR (300 MHz, DMSO-d₆) δ 9.27 (s, 2H), 9.19 (s, 2H), 9.09 (s, 2H), 9.03 (s, 2H), 7.44 (dd, J=9.5, 2.8 Hz, 1H), 7.27 (d, J=8.7 Hz, 1H), 7.24 (d, J=2.8 Hz, 1H), 7.19-7.10 (m, 1H), 6.97 (dd, J=8.7, 2.8 Hz, 1H), 6.89 (dd, J=8.7, 5.6 Hz, 1H), 4.61 (s, 2H), 4.53 (s, 2H), 3.76 (s, 3H); MS (ES+)m/z 411.1 (M+1).

### Example 1.7

### Synthesis of 2-[2-(2-carbamimidoylsulfanylmethyl-5-chloro-phenylsulfanyl)-5-fluoro-benzyl]-isothiourea dihydrobromide

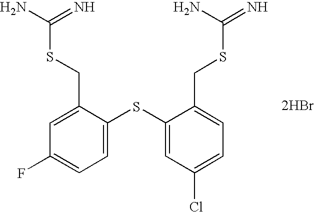

Following the procedure as described in Example 1, making non-critical variations using (4-chloro-2-(4-fluoro-2-(hydroxymethyl)phenylthio)phenyl)methanol to replace 1-(1-hydroxyethyl)-2-(6-(1-hydroxyethyl)phenoxy)benzene, 2-[2-(2-Carbamimidoylsulfanylmethyl-5-chloro-phenylsulfanyl)-5-fluoro-benzyl]-isothiourea dihydrobromide was obtained as a colorless solid in 82% yield: ¹H NMR (300 MHz, DMSO-d₆) δ 9.24 (s, 4H), 9.08 (s, 4H), 7.45-7.73 (m, 2H), 7.25-7.15 (m, 3H), 6.83 (d, J=2.2 Hz, 1H), 4.62 (s, 2H), 4.59 (s, 2H); MS (ES+) m/z 415.1 (M+1).

### Example 1.8

### Synthesis of 2-[2-(2-carbamimidoylsulfanylmethyl-6-methylphenylsulfanyl)-benzyl]isothiourea dihydrobromide

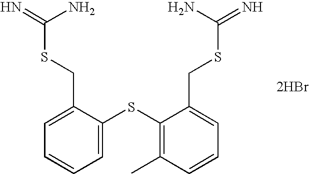

Following the procedure as described in Example 1, making non-critical variations using (2-(2-(hydroxymethyl)-6-methylphenylthio)phenyl)methanol to replace 1-(1-hydroxyethyl)-2-(6-(1-hydroxyethyl)phenoxy)benzene, 2-[2-(2-carbamimidoylsulfanylmethyl-6-methylphenylsulfanyl)-benzyl]isothiourea dihydrobromide was obtained as a colorless solid in 93% yield: ¹H NMR (300 MHz, DMSO-d₆) δ 9.27 (s, 2H), 9.17 (s, 2H), 9.10 (s, 2H), 9.00 (s, 2H), 7.56-7.34 (m, 4H), 7.26-7.11 (m, 2H), 6.50-6.30 (m, 1H), 4.67 (s, 2H), 4.61 (s, 2H), 2.18 (s, 3H); MS (ES+) m/z 377.1 (M+1).

### Example 1.9

### Synthesis of 2-[2-(2-carbamimidoylsulfanylmethyl-4,5-difluorophenylsulfanyl)-benzyl]isothiourea dihydrobromide

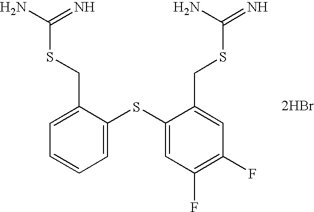

Following the procedure as described in Example 1, making non-critical variations using (4,5-difluoro-2-(2-(hydroxymethyl)phenylthio)phenyl)methanol to replace 1-(1-hydroxyethyl)-2-(6-(1-hydroxyethyl)phenoxy)benzene, 2-[2-(2-carbamimidoylsulfanylmethyl-4,5-difluorophenylsulfanyl)benzyl]isothiourea dihydrobromide was obtained as a colorless solid in 94% yield: ¹H NMR (300 MHz, DMSO-d₆) δ 9.27 (s, 2H), 9.23 (s, 2H), 9.09 (s, 2H), 9.06 (s, 2H), 7.77 (dd, J=11.2, 8.2 Hz, 1H), 7.59 (dd, J=7.4, 1.6 Hz, 1H), 7.37 (ddd, J=8.9, 7.3, 1.5 Hz, 2H), 7.19 (dd, J=7.5, 1.6 Hz, 1H), 7.09 (dd, J=10.6, 7.9 Hz, 1H), 4.61 (s, 2H), 4.60 (s, 2H); MS (ES+) m/z 399.1 (M+1).

### Example 1.10

### Synthesis of 2-[2-(2-carbamimidoylsulfanylmethyl-4-methylphenylsulfanyl)-benzyl]isothiourea dihydrobromide

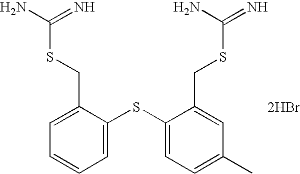

Following the procedure as described in Example 1, making non-critical variations using (2-(2-(hydroxymethyl)-4-methylphenylthio)phenyl)methanol to replace 1-(1-hydroxyethyl)-2-(6-(1-hydroxyethyl)phenoxy)benzene, 2-[2-(2-carbamimidoylsulfanylmethyl-4-methylphenylsulfanyl)-benzyl]isothiourea dihydrobromide was obtained as a colorless solid in 93% yield: ¹H NMR (300 MHz, DMSO-d₆) δ 9.20 (s, 4H), 9.04 (s, 4H), 7.52 (dd, J=5.9, 3.2 Hz, 1H), 7.41 (d, J=1.6 Hz, 1H), 7.28 (m, 2H), 7.18 (dd, J=8.2, 1.5 Hz, 1H), 7.10 (d, J=8.0 Hz, 1H), 6.93 (m, 1H), 4.60 (s, 2H), 4.53 (s, 2H), 2.28 (s, 3H); MS (ES+) m/z 377.1 (M+1).

### Example 1.11

### Synthesis of 2-[2-(2-carbamimidoylsulfanylmethyl-5-chlorophenylsulfanyl)-benzyl]isothiourea dihydrobromide

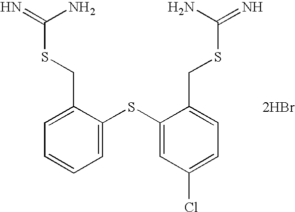

Following the procedure as described in Example 1, making non-critical variations using (4-chloro-2-(2-(hydroxymethyl)phenylthio)phenyl)methanol to replace 1-(1-hydroxyethyl)-2-(6-(1-hydroxyethyl)phenoxy)benzene, 2-[2-(2-carbamimidoylsulfanylmethyl-5-chlorophenylsulfanyl)benzyl]isothiourea dihydrobromide was obtained as a colorless solid in 53% yield: ¹H NMR (300 MHz, DMSO-d₆) δ 9.25 (s, 2H), 9.21 (s, 2H), 9.07 (s, 2H), 9.04 (s, 2H), 7.63 (dd, J=7.4, 1.6 Hz, 1H), 7.57 (d, J=8.3 Hz, 1H), 7.46 (dd, J=7.4, 1.6 Hz, 1H), 7.40 (ddd, J=7.3, 4.3, 1.7 Hz, 2H), 7.28 (dd, J=7.5, 1.6 Hz, 1H), 6.87 (d, J=2.2 Hz, 1H), 4.61 (s, 2H), 4.60 (s, 2H); MS (ES+) m/z 397.1 (M+1).

### Example 1.12

### Synthesis of 2-[2-(2-carbamimidoylsulfanylmethyl-5-fluorophenylsulfanyl)benzyl]-isothiourea dihydrobromide

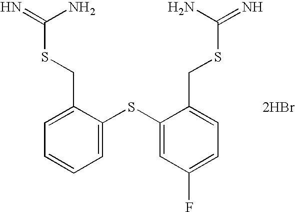

Following the procedure as described in Example 1, making non-critical variations using (4-fluoro-2-(2-(hydroxymethyl)phenylthio)phenyl)methanol to replace 1-(1-hydroxyethyl)-2-(6-(1-hydroxyethyl)phenoxy)benzene, 2-[2-(2-carbamimidoylsulfanylmethyl-5-fluorophenylsulfanyl)benzyl]isothiourea dihydrobromide was obtained as a colorless solid in 53% yield: ¹H NMR (300 MHz, DMSO-d₆) δ 9.24 (s, 2H), 9.21 (s, 2H), 9.07 (s, 2H), 9.04 (s, 2H), 7.62 (m, 2H), 7.44-7.39 (m, 2H), 7.30 (dd, J=1.5, 7.6 Hz, 1H), 7.14-7.09 (m, 1H), 6.66 (dd, J=9.2, 2.7 Hz, 1H), 4.61 (s, 2H), 4.60 (s, 2H); MS (ES+) m/z 381.1 (M+1).

### Example 1.13

### Synthesis of 2-[2-(1-carbamimidoylsulfanylmethylnaphthalen-2-ylsulfanyl)benzyl]-isothiourea dihydrobromide

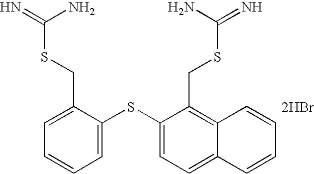

Following the procedure as described in Example 1, making non-critical variations using (2-(1-(hydroxymethyl)naphthalen-2-ylthio)phenyl)methanol to replace 1-(1-hydroxyethyl)-2-(6-(1-hydroxyethyl)phenoxy)benzene, 2-[2-(1-carbamimidoylsulfanylmethylnaphthalen-2-ylsulfanyl)benzyl]isothiourea dihydrobromide was obtained as a colorless solid in 24% yield: ¹H NMR (300 MHz, DMSO-d₆) δ 9.34 (s, 2H), 9.22 (s, 2H), 9.13 (s, 2H), 9.04 (s, 2H), 8.16 (dd, J=11.7, 5.3 Hz, 2H), 8.03 (dd, J=6.8, 2.5 Hz, 1H), 7.83 (d, J=8.5 Hz, 1H), 7.62-7.56 (m, 2H), 7.50 (dd, J=1.2, 7.7 Hz, 1H), 7.09-7.05 (m, 1H), 7.01-6.95 (m, 1H), 6.21 (dd, J=1.2, 7.7 Hz, 1H), 4.86 (s, 2H), 4.81 (s, 2H); MS (ES+) m/z 413.1 (M+1).

### Example 1.14

### Synthesis of 2-[2-(1-carbamimidoylsulfanylmethylnaphthalen-2-ylsulfanyl)-5-fluorobenzyl]isothiourea dihydrobromide

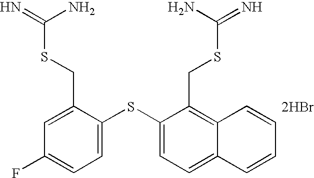

Following the procedure as described in Example 1, making non-critical variations using (5-fluoro-2-(1-(hydroxymethyl)naphthalen-2-ylthio)phenyl)methanol to replace 1-(1-hydroxyethyl)-2-(6-(1-hydroxyethyl)phenoxy)benzene, 2-[2-(1-carbamimidoylsulfanylmethylnaphthalen-2-ylsulfanyl)-5-fluorobenzyl]isothiourea dihydrobromide was obtained as a colorless solid in 92% yield: ¹H NMR (300 MHz, DMSO-d₆) δ 9.38 (s, 2H), 9.22 (s, 2H), 9.17 (s, 2H), 9.05 (s, 2H), 8.15 (dd, J=12.9, 6.6 Hz, 2H), 7.90-8.10 (m, 1H), 7.82 (d, J=8.5 Hz, 1H), 7.70-7.50 (m, 2H), 7.46 (dd, J=9.4, 2.8 Hz, 1H), 6.98-6.88 (m, 1H), 6.24 (dd, J=8.5, 5.4 Hz, 1H), 4.86 (s, 2H), 4.81 (s, 2H); MS (ES+) m/z 431.1 (M+1).

### Example 1.15

### Synthesis of 2-[2-(2-carbamimidoylsulfanylmethyl-5-fluorophenylsulfanyl)-5-fluorobenzyl]isothiourea dihydrobromide

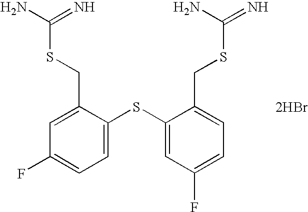

Following the procedure as described in Example 1, making non-critical variations using ((4-fluoro-2-(4-fluoro-2-(hydroxymethyl)phenylthio)phenyl)methanol to replace 1-(1-hydroxyethyl)-2-(6-(1-hydroxyethyl)phenoxy)benzene, 2-[2-(2-carbamimidoylsulfanylmethyl-5-fluorophenylsulfanyl)-5-fluorobenzyl]isothiourea dihydrobromide was obtained as a colorless solid in 82% yield: ¹H NMR (300 MHz, DMSO-d₆) δ 9.23 (s, 4H), 9.07 (s, 4H), 7.64-7.52 (m, 2H), 7.44 (dd, J=8.7, 5.7 Hz, 1H), 7.32-7.24 (m, 1H), 7.19-7.08 (m, 1H), 6.61 (dd, J=9.2, 2.6 Hz, 1H), 4.62 (s, 2H), 4.58 (s, 2H); MS (ES+) m/z 399.1 (M+1).

### Example 1.16

### Synthesis of 2-[2-(2-carbamimidoylsulfanylmethyl-4,5-difluorophenylsulfanyl)-5-fluorobenzyl]isothiourea dihydrobromide

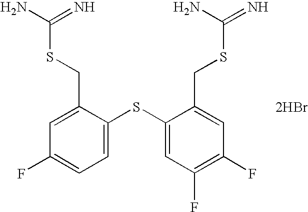

Following the procedure as described in Example 1, making non-critical variations using (4,5-difluoro-2-(4-fluoro-2-(hydroxymethyl)phenylthio)phenyl)methanol to replace 1-(1-hydroxyethyl)-2-(6-(1-hydroxyethyl)phenoxy)benzene, 2-[2-(2-carbamimidoylsulfanylmethyl-4,5-difluorophenylsulfanyl)-5-fluorobenzyl]isothiourea dihydrobromide was obtained as a colorless solid in 94% yield: ¹H NMR (300 MHz, DMSO-d₆) δ 9.24 (s, 4H), 9.08 (s, 4H), 7.74 (dd, J=11.2, 8.2 Hz, 1H), 7.53 (dd, J=9.5, 2.7 Hz, 1H), 7.35 (dd, J=8.7, 5.7 Hz, 1H), 7.30-7.21 (m, 1H), 7.04 (dd, J=10.6, 7.8 Hz, 1H), 4.59 (d, J=1.5 Hz, 4H); MS (ES+) m/z 417.1 (M+1).

### Example 1.17

### Synthesis of 2-[2-(2-carbamimidoylsulfanylmethyl-4-fluorophenylsulfanyl)-3-nitrobenzyl]isothiourea dihydrobromide

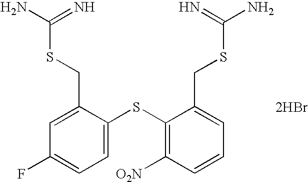

Following the procedure as described in Example 1, making non-critical variations using (5-fluoro-2-(2-(hydroxymethyl)-6-nitrophenylthio)phenyl)methanol to replace 1-(1-hydroxyethyl)-2-(6-(1-hydroxyethyl)phenoxy)benzene, 2-[2-(2-carbamimidoylsulfanylmethyl-4,5-difluorophenylsulfanyl)-5-fluorobenzyl]isothiourea dihydrobromide was obtained as a colorless solid in 86% yield: ¹H NMR (300 MHz, DMSO-d₆) δ 9.49-8.85 (m, 8H), 8.01-7.86 (m, 2H), 7.78-7.70 (m, 1H), 7.45 (dd, J=9.4, 2.8 Hz, 1H), 7.18-7.08 (m, 1H), 6.81 (dd, J=8.8, 5.4 Hz, 1H), 4.63 (s, 2H), 4.61 (s, 2H); MS (ES+) m/z 426.1 (M+1).

### Example 1.18

### Synthesis of 2-[2-(2-carbamimidoylsulfanylmethyl-4-fluorophenylsulfanyl)-5-(trifluoromethyl)benzyl]isothiourea dihydrobromide

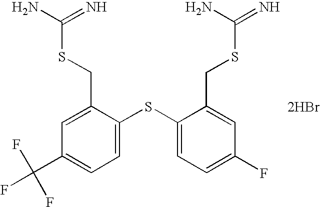

Following the procedure as described in Example 1, making non-critical variations using (5-fluoro-2-(2-(hydroxymethyl)-4-(trifluoromethyl)phenylthio)-phenyl)methanol to replace 1-(1-hydroxyethyl)-2-(6-(1-hydroxyethyl)phenoxy)benzene, 2-[2-(2-carbamimidoylsulfanylmethyl-4-fluorophenylsulfanyl)-5-(trifluoromethyl)benzyl]isothiourea dihydrobromide was obtained as a colorless solid in 88% yield: ¹H NMR (300 MHz, DMSO-d₆) δ 9.42-8.92 (m, 8H), 7.92 (d, J=1.4 Hz, 1H), 7.65-7.50 (m, 3H), 7.39-7.28 (m, 1H), 6.88 (d, J=8.4 Hz, 1H), 4.71 (s, 2H), 4.56 (s, 2H); ¹³C NMR (75 MHz, DMSO-d₆) δ 169.0, 168.9, 164.7, 161.4, 143.0, 141.6, 139.3, 134.1, 129.7, 127.1, 122.4, 119.0, 118.7, 118.1, 117.9, 33.7, 33.3; ¹⁹F NMR (300 MHz, DMSO-d₆) δ−60.9, −109.7; MS (ES+) m/z 449.1 (M+1).

### Example 1.19

### Synthesis of 2-[2-(2-carbamimidoylsulfanylmethylphenylsulfanyl)-5-aminobenzyl]-isothiourea trihydrobromide

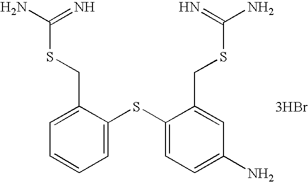

Following the procedure as described in Example 1, making non-critical variations using (5-amino-2-(2-(hydroxymethyl)phenylthio)phenyl)methanol to replace 1-(1-hydroxyethyl)-2-(6-(1-hydroxyethyl)phenoxy)benzene, 2-[2-(2-carbamimidoylsulfanylmethylphenylsulfanyl)-5-aminobenzyl]-isothiourea trihydrobromide was obtained as a colorless solid in 97% yield: ¹H NMR (300 MHz, DMSO-d₆) δ 9.45-8.81 (m, 8H), 8.92 (s, 1H), 7.60-7.41 (m, 2H), 7.32-7.20 (m, 3H), 7.17-7.07 (m, 2H), 6.98-6.81 (m, 2H), 4.70-4.53 (m, 2H), 4.50 (s, 2H); MS (ES+) m/z 378.1 (M+1).

### Example 1.20

### Synthesis of 2-[2-(2-carbamimidoylsulfanylmethyl-4-chlorophenylsulfanyl)-5-fluorobenzyl]isothiourea dihydrobromide

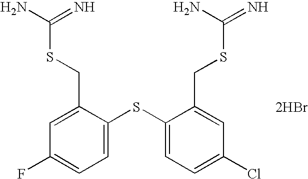

Following the procedure as described in Example 1, making non-critical variations using (5-chloro-2-(4-fluoro-2-(hydroxymethyl)phenylthio)phenyl)methanol to replace 1-(1-hydroxyethyl)-2-(6-(1-hydroxyethyl)phenoxy)benzene, 2-[2-(2-carbamimidoylsulfanylmethyl-4-chlorophenylsulfanyl)-5-fluorobenzyl]isothiourea dihydrobromide was obtained as a colorless solid in 88% yield: ¹H NMR (300 MHz, DMSO-d₆) δ 9.24 (d, J=10.0 Hz, 4H), 9.07 (d, J=10.0 Hz, 4H), 7.66 (d, J=2.7 Hz, 1H), 7.53 (dd, J=9.5, 2.7 Hz, 1H), 7.39-7.21 (m, 3H), 6.94 (d, J=8.5 Hz, 1H), 4.60 (s, 2H), 4.57 (s, 2H); MS (ES+) m/z 415.1 (M+1).

### Example 1.21

### Synthesis of 2-[2-(2-carbamimidoylsulfanylmethylphenylsulfanyl)-5-ethylaminobenzyl]isothiourea trihydrobromide

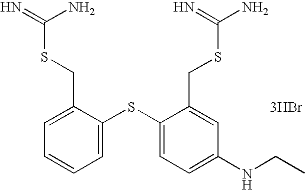

Following the procedure as described in Example 1, making non-critical variations using (2-{[4-(ethylamino)-2-(hydroxymethyl)phenyl]thio}phenyl)methanol to replace 1-(1-hydroxyethyl)-2-(6-(1-hydroxyethyl)phenoxy)benzene, 2-[2-(2-carbamimidoylsulfanylmethylphenylsulfanyl)-5-ethylaminobenzyl]isothiourea trihydrobromide was obtained as a colorless solid in 90% yield: ¹H NMR (300 MHz, DMSO-d₆) δ 9.35-8.83 (m, 8H), 7.50-7.09 (m, 6H), 6.90 (s, 1H), 6.77-6.64 (m, 2H), 4.60 (s, 2H), 4.42 (s, 2H), 3.07 (q, J=7.1 Hz, 2H), 1.14 (t, J=7.1 Hz, 3H); MS (ES+) m/z 406.1 (M+1).

### Example 1.22

### Synthesis of 2-[2-(2-carbamimidoylsulfanylmethyl-4-chlorophenylsulfanyl)-5-chlorobenzyl]isothiourea dihydrobromide

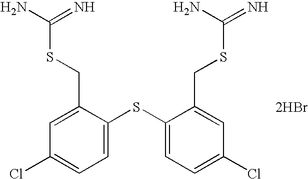

Following the procedure as described in Example 1, making non-critical variations using 5-chloro-1-(hydroxymethyl)-2-((4-chloro-6-(hydroxymethyl)phenyl)thio)benzene to replace 1-(1-hydroxyethyl)-2-(6-(1-hydroxyethyl)phenoxy)benzene, 2-[2-(2-carbamimidoylsulfanylmethyl-4-chlorophenylsulfanyl)-5-chlorobenzyl]isothiourea dihydrobromide was obtained as a colorless solid in 93% yield: ¹H NMR (300 MHz, DMSO-d₆) δ 9.24 (s, 4H), 9.08 (s, 4H), 7.69 (d, J=2.4 Hz, 2H), 7.41 (dd, J=8.5, 2.4 Hz, 2H), 7.11 (d, J=8.5 Hz, 2H), 4.58 (s, 4H); MS (ES+) m/z 431.1 (M+1), 433.1 (M+1).

### Example 1.23

### Synthesis of 2-[2-(2-carbamimidoylsulfanylethyl-4-fluorophenylsulfanyl)-5-fluorobenzyl]isothiourea dihydrobromide

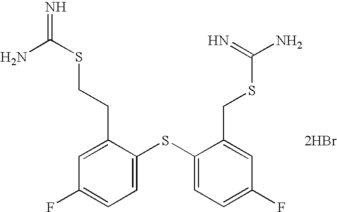

Following the procedure as described in Example 1, making non-critical variations using 2-(5-fluoro-2-{[4-fluoro-2-(hydroxymethyl)phenyl]thio}phenyl)ethanol to replace 1-(1-hydroxyethyl)-2-(6-(1-hydroxyethyl)phenoxy)benzene, 2-[2-(2-carbamimidoylsulfanylethyl-4-fluorophenylsulfanyl)-5-fluorobenzyl]isothiourea dihydrobromide was obtained as a colorless solid in 81% yield: ¹H NMR (300 MHz, DMSO-d₆) δ 9.45-8.75 (m, 8H), 7.49 (dd, J=9.5, 2.7 Hz, 1H), 7.36 (d, J=9.5 Hz, 1H), 7.24-7.03 (m, 4H), 4.57 (s, 2H), 3.45 (t, J=7.2 Hz, 2H), 3.05 (t, J=7.2 Hz, 2H); MS (ES+) m/z 413.1 (M+1).

### Example 1.24

### Synthesis of 2-[2-(2-carbamimidoylsulfanylethyl-4-chlorophenylsulfanyl)-5-fluorobenzyl]isothiourea dihydrobromide

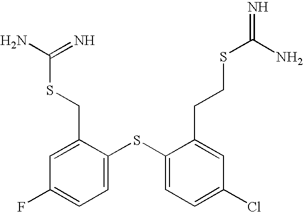

Following the procedure as described in Example 1, making non-critical variations using 2-(5-chloro-2-(4-fluoro-2-(hydroxymethyl)phenylthio)phenyl)ethanol to replace 1-(1-hydroxyethyl)-2-(6-(1-hydroxyethyl)phenoxy)benzene, 2-[2-(2-carbamimidoylsulfanylethyl-4-chlorophenylsulfanyl)-5-fluorobenzyl]isothiourea dihydrobromide was obtained as a colorless solid in 74% yield: ¹H NMR (300 MHz, DMSO-d₆) δ 9.36-8.85 (m, 8H), 7.64-7.42 (m, 2H), 7.32-7.20 (m, 3H), 6.90 (d, J=8.5 Hz, 1H), 4.56 (s, 2H), 3.48 (t, J=7.3 Hz, 2H), 3.05 (t, J=7.3 Hz, 2H); ¹³C NMR (75 MHz, DMSO-d₆) δ 169.7, 169.0, 140.9, 139.4, 136.7, 134.0, 132.7, 132.6, 130.4, 129.6, 129.6, 128.6, 33.6, 32.1, 30.4 MS (ES+) m/z 429.1 (M+1), 431.1 (M+1).

### Example 1.25

### Synthesis of 2-[2-(2-carbamimidoylsulfanylethylphenylsulfanyl)benzyl]isothiourea dihydrobromide

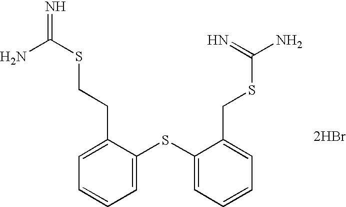

Following the procedure as described in Example 1, making non-critical variations using 2-(2-(2-(hydroxymethyl)phenylthio)phenyl)ethanol to replace 1-(1-hydroxyethyl)-2-(6-(1-hydroxyethyl)phenoxy)benzene, 2-[2-(2-carbamimidoylsulfanylethylphenylsulfanyl)benzyl]isothiourea dihydrobromide was obtained as a colorless solid in 97% yield: ¹H NMR (300 MHz, DMSO-d₆) δ 9.50-7.80 (m, 8H), 7.63-7.49 (m, 1H), 7.42 (dd, J=7.7, 1.2 Hz, 1H), 7.37-7.20 (m, 4H), 7.08 (dd, J=7.7, 1.2 Hz, 1H), 7.03-6.96 (m, 1H), 4.60 (s, 2H), 3.45 (t, J=7.3 Hz, 2H), 3.04 (t, J=7.2 Hz, 2H); ¹³C NMR (75 MHz, DMSO-d₆) δ 169.9, 169.4, 140.1, 135.6, 134.9, 133.6, 133.0, 132.2, 131.5, 131.0, 130.2, 128.8, 128.5, 33.9, 32.3, 30.9; MS (ES+) m/z 377.1 (M+1).

### Example 1.26

### Synthesis of 2-[2-(2-carbamimidoylsulfanylmethylphenylsulfanyl)-5-chlorobenzyl]-isothiourea dihydrobromide

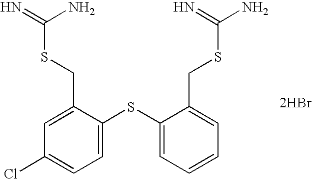

Following the procedure as described in Example 1, making non-critical variations using (5-chloro-2-(2-(hydroxymethyl)phenylthio)phenyl)methanol to replace 1-(1-hydroxyethyl)-2-(6-(1-hydroxyethyl)phenoxy)benzene, 2-[2-(2-carbamimidoylsulfanylmethylphenylsulfanyl)-5-chlorobenzyl]isothiourea dihydrobromide was obtained as a colorless solid in 94% yield: ¹H NMR (300 MHz, DMSO-d₆) δ 9.48-8.93 (m, 8H), 7.72 (d, J=2.1 Hz, 1H), 7.63-7.56 (m, 1H), 7.43-7.29 (m, 3H), 7.21-7.12 (m, 1H), 7.02 (d, J=8.5 Hz, 1H), 4.66-4.59 (m, 4H); ¹³C NMR (75 MHz, DMSO-d₆) δ 169.3, 169.0, 137.5, 136.1, 134.7, 134.3, 134.2, 133.9, 132.9, 131.7, 130.9, 130.5, 130.1, 129.4, 33.9, 33.5; MS (ES+) m/z 399.1 (M+1).

### Example 1.27

### Synthesis of 2-[2-(2-carbamimidoylsulfanylmethylphenylsulfanyl)-5-(trifluoromethyl)benzyl]isothiourea dihydrobromide

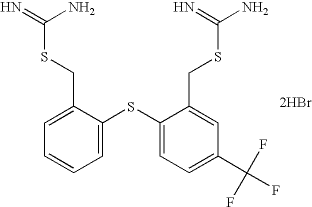

Following the procedure as described in Example 1, making non-critical variations using (2-(2-(hydroxymethyl)-4-(trifluoromethyl)phenylthio)phenyl)methanol to replace 1-(1-hydroxyethyl)-2-(6-(1-hydroxyethyl)phenoxy)benzene, 2-[2-(2-carbamimidoylsulfanylmethylphenylsulfanyl)-5-(trifluoromethyl)benzyl]isothiourea dihydrobromide was obtained as a colorless solid in 94% yield: ¹H NMR (300 MHz, DMSO-d₆) δ 9.35-9.06 (m, 8H), 7.95 (s, 1H), 7.70-7.40 (m, 5H), 6.93 (d, J=7.7 Hz, 1H), 4.74 (s, 2H), 4.60 (s, 2H); ¹³C NMR (75 MHz, DMSO-d₆) δ 169.2, 169.0, 142.9, 138.1, 136.3, 134.6, 132.0, 132.0, 131.8, 130.8, 130.3, 127.7, 126.6, 33.9, 33.3; MS (ES+) m/z 431.1 (M+1).

### Example 1.28

### Synthesis of 2,2′-(methylazanediyl)bis(2,1-phenylene)bis-(methylene)dicarbamimidothioate trihydrobromide

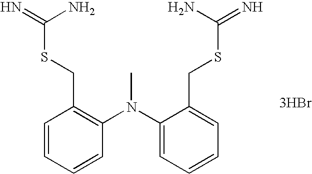

Following the procedure as described in Example 1, making non-critical variations using 1-(hydroxymethyl)-2-((6-hydroxymethylphenyl)(methyl)amino)benzene to replace 1-(1-hydroxyethyl)-2-(6-(1-hydroxyethyl)phenoxy)benzene, 2,2′-(methylazanediyl)bis(2,1-phenylene)bis(methylene)dicarbamimidothioate trihydrobromide was obtained as a colorless solid in 75% yield: ¹H NMR (300 MHz, DMSO-d₆) δ 9.40-8.67 (m, 8H), 7.52-6.76 (m, 8H), 5.74 (s, 1H), 4.17 (s, 4H), 3.16 (s, 3H); MS (ES+) m/z 360.2 (M+1).

### Example 1.29

### Synthesis of 2-[2-(2-methylcarbamimidoylsulfanylmethylphenylsulfanyl)benzyl]-methylisothiourea dihydrobromide

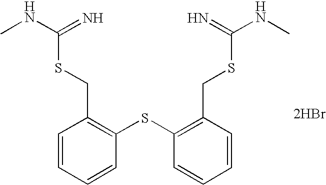

Following the procedure as described in Example 1, making non-critical variations using 1-(hydroxymethyl)-2-((6-(hydroxymethyl)phenyl)thio)benzene to replace 1-(1-hydroxyethyl)-2-(6-(1-hydroxyethyl)phenoxy)benzene and to use methylthiourea to replace thiourea, 2-[2-(2-methylcarbamimidoylsulfanylmethylphenylsulfanyl)benzyl]-methylisothiourea dihydrobromide was obtained as a colorless solid in 97% yield: ¹H NMR (300 MHz, DMSO-d₆) δ 9.74 (q, J=5.1 Hz, 2H), 9.48 (s, 2H), 9.15 (s, 2H), 7.58-7.55 (m, 2H), 7.36-7.27 (m, 4H), 7.09-7.06 (m, 2H), 4.67 (s, 4H), 2.87 (d, J=5.1 Hz, 6H); ¹³C NMR (75 MHz, DMSO-d₆) δ 165.9, 136.0, 135.1, 133.2, 131.5, 130.3, 128.9, 34.5, 31.2; MS (ES+) m/z 391.1 (M+1).

### Example 1.30

### Synthesis of 2-[2-(2-carbamimidoylsulfanylmethyl-4-fluorophenylsulfanyl)-5-(methylsulfonyl)benzyl]isothiourea dihydrobromide

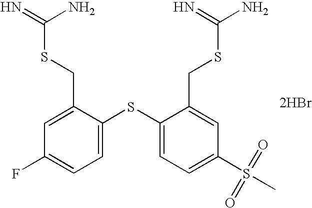

Following the procedure as described in Example 1, making non-critical variations using (5-fluoro-2-(2-(hydroxymethyl)-4-(methylsulfonyl)phenylthio)-phenyl)methanol to replace 1-(1-hydroxyethyl)-2-(6-(1-hydroxyethyl)phenoxy)benzene, 2-[2-(2-carbamimidoylsulfanylmethyl-4-fluorophenylsulfanyl)-5-(methylsulfonyl)benzyl]isothiourea dihydrobromide was obtained as a colorless solid in 75% yield: ¹H NMR (300 MHz, DMSO-d₆) δ 9.36 (s, 2H), 9.27 (s, 2H), 9.18 (s, 2H), 9.08 (s, 2H), 8.08 (d, J=2.0 Hz, 1H), 7.72 (dd, J=8.5, 2.0 Hz, 1H), 7.66 (dd, J=9.5, 2.9 Hz, 1H), 7.58 (dd, J=8.5, 5.7 Hz, 1H), 7.42-7.30 (m, 1H), 6.86 (d, J=8.5 Hz, 1H), 4.75 (s, 2H), 4.60 (s, 2H), 3.17 (s, 3H); ¹³C NMR (75 MHz, DMSO-d₆) δ 169.0, 163.2 (d, J_{C-F}=249.6 Hz), 144.6, 142.0 (d, J_{C-F}=8.6 Hz), 139.7 (d, J_{C-F}=9.2 Hz), 139.0, 133.8, 129.3, 128.2, 126.7, 126.7 (d, J_{C-F}=3.4 Hz), 119.0 (d, J_{C-F}=23.3 Hz), 118.1 (d, J_{C-F}=21.4 Hz), 44.1, 33.8, 33.4; MS (ES+) m/z 459.1 (M+1).

### Example 1.31

### Synthesis of 2-[2-(2-carbamimidoylsulfanylmethylphenylsulfanyl)benzyl]-isothiourea dihydrobromide

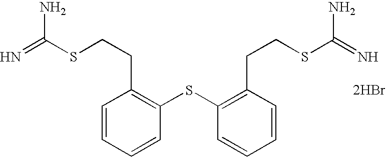

Following the procedure as described in Example 1, making non-critical variations using 1-(2-hydroxyethyl)-2-((6-(2-hydroxyethyl)phenyl)thio)benzene to replace 1-(1-hydroxyethyl)-2-(6-(1-hydroxyethyl)phenoxy)benzene, 2-[2-(2-carbamimidoylsulfanylmethylphenylsulfanyl)benzyl]-isothiourea dihydrobromide was obtained as a colorless solid in 79% yield: ¹H NMR (300 MHz, DMSO-d₆) δ 9.13 (br s, 4H), 9.00 (br s, 4H), 7.40 (d, J=7.5 Hz, 2H), 7.32-7.22 (m, 2H), 7.23-7.11 (m, 2H), 6.99 (dd, J=7.5, 1.0 Hz, 2H), 3.45 (t, J=7.2 Hz, 4H), 3.03 (t, J=7.2 Hz, 4H); ¹³C NMR (75 MHz, DMSO-d₆) δ 169.9, 139.5, 134.2, 132.2, 130.9, 128.7, 128.4, 32.3, 30.9; MS (ES+) m/z 391.1 (M+1).

### Example 2

### Synthesis of 2-[2-(2-carbamimidoylsulfanylmethyl-4-fluoro-phenylsulfanyl)-5-fluoro-benzyl]-isothiourea dihydrobromide

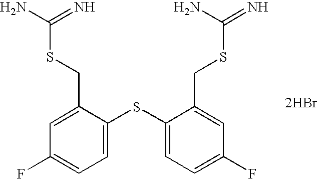

Thiourea (0.42 g, 5.54 mmol) was added to the stirred solution of bis(2-(bromomethyl)-4-fluorophenyl)sulfane (1.13 g, 2.77 mmol) in ethanol (35.0 mL). The mixture was stirred at 80° C. for 16 h and evaporated to dryness. The white residue was crystallized from ethanol/acetonitrile (1/5) to afford 2-[2-(2-carbamimidoylsulfanylmethyl-4-fluoro-phenylsulfanyl)-5-fluoro-benzyl]-isothiourea dihydrobromide in 82% yield (0.91 g): ¹H NMR (300 MHz, DMSO-d₆) δ 9.25 (br s, 4H), 9.08 (br s, 4H), 7.50 (dd, J=9.5, 2.7 Hz, 2H), 7.26-7.19 (m, 2H), 7.16 (dd, J=8.8, 5.8 Hz, 2H), 4.60 (s, 4H); ¹³C NMR (75 MHz, DMSO-d₆) δ 169.1, 161.9 (d, J_{C-F}=246.9 Hz), 138.5 (d, J_{C-F}=8.3 Hz), 135.7 (d, J_{C-F}=8.6 Hz), 130.9 (d, J_{C-F}=3.1 Hz), 118.4 (d, J_{C-F}=23.3 Hz), 117.5 (d, J_{C-F}=21.8 Hz), 33.7; MS (ES+) m/z 399.1 (M+1).

### Example 2.1

### Synthesis of 2-{[2-({[amino(imino)methyl]thio}methyl)-4-fluorophenyl]thio}-5-nitrobenzyl imidothiocarbamate dihydrobromide

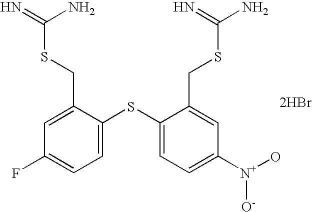

Following the procedure as described in Example 2, making non-critical variations using (2-(bromomethyl)-4-fluorophenyl)(2-(bromomethyl)-4-nitrophenyl)sulfane to replace bis(2-(bromomethyl)-4-fluorophenyl)sulfane, 2-{[2-({[Amino(imino)methyl]thio}methyl)-4-fluorophenyl]thio}-5-nitrobenzyl imidothiocarbamate dihydrobromide was obtained as a colorless solid in 90% yield (0.56 g): ¹H NMR (300 MHz, DMSO-d₆) δ 9.52-8.99 (m, 8H), 8.43 (d, J=2.5 Hz, 1H), 8.02 (dd, J=8.8, 2.5 Hz, 1H), 7.71-7.61 (m, 2H), 7.43-7.29 (m, 1H), 6.81 (d, J=8.8 Hz, 1H), 4.80 (s, 2H), 4.58 (s, 2H); ¹³C NMR (75 MHz, DMSO-d₆) δ 168.9, 163.5 (d, J_{C-F}=250.7 Hz), 147.2, 145.7, 142.3 (d, J_{C-F}=8.7 Hz), 140.2 (d, J_{C-F}=9.6 Hz), 133.7, 128.8, 126.0 (d, J_{C-F}=3.1 Hz), 125.5, 124.5, 119.2 (d, J_{C-F}=22.9 Hz), 118.2 (d, J_{C-F}=21.8 Hz, 1C), 33.8, 33.1; MS (ES+) m/z 426.1 (M+1).

### Example 2.2

### Synthesis of 2-({2-({[amino(imino)methyl]thio}methyl)-4-[(dimethylamino)sulfonyl]phenyl}thio)-5-fluorobenzyl imidothiocarbamate dihydrobromide

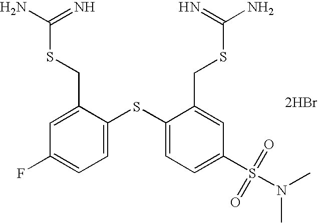

Following the procedure as described in Example 2, making non-critical variations using 3-bromomethyl-4-(2-bromomethyl-4-fluorophenylsulfanyl)-N,N-dimethylbenzenesulfonamide to replace bis(2-(bromomethyl)-4-fluorophenyl)sulfane, 2-({2-({[amino(imino)methyl]thio}methyl)-4-[(dimethylamino)sulfonyl]phenyl}thio)-5-fluorobenzyl imidothiocarbamate dihydrobromide was obtained as a colorless solid in 97% yield: ¹H NMR (300 MHz, DMSO-d₆) δ 9.32 (br s, 2H), 9.22 (br s, 2H), 9.14 (br s, 2H), 9.05 (br s, 2H), 7.90 (d, J=1.9 Hz, 1H), 7.65-7.55 (m, 3H), 7.41-7.31 (m, 1H), 6.88 (d, J=8.5 Hz, 1H), 4.73 (s, 2H), 4.55 (s, 2H), 2.56 (s, 6H); MS (ES+) m/z 488.1 (M+1).

### Example 2.3

### Synthesis of 2-[2-(2-carbamimidoylsulfanylmethylphenylsulfonyl)benzyl]-isothiourea dihydrobromide

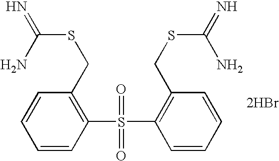

Following the procedure as described in Example 2, making non-critical variations using 2,2′-sulfonylbis((bromomethyl)benzene) to replace bis(2-(bromomethyl)-4-fluorophenyl)sulfane, 2-[2-(2-carbamimidoylsulfanylmethylphenylsulfonyl)benzyl]-isothiourea dihydrobromide was obtained as a colorless solid in 49% yield: ¹H NMR (300 MHz, DMSO-d₆) δ 9.15 (br s, 4H), 9.02 (br s, 4H), 8.07 (dd, J=8.0, 1.0 Hz, 2H), 7.85-7.70 (m, 2H), 7.70-7.60 (m, 4H), 4.62 (s, 4H); MS (ES+) m/z 395.1 (M+1).

### Example 2.4

### Synthesis of 2-[2-(2-carbamimidoylsulfanylmethyl-4-fluorophenylsulfonyl)-5-fluorobenzyl]isothiourea dihydrobromide

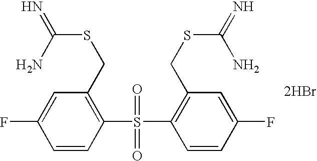

Following the procedure as described in Example 2, making non-critical variations using 4,4′-sulfonylbis(3-(bromomethyl)-1-fluorobenzene) to replace bis(2-(bromomethyl)-4-fluorophenyl)sulfane, 2-[2-(2-carbamimidoylsulfanylmethyl-4-fluorophenylsulfonyl)-5-fluorobenzyl]isothiourea dihydrobromide was obtained as a colorless solid in 92% yield: ¹H NMR (300 MHz, DMSO-d₆) δ 9.13 (br s, 4H), 9.02 (br s, 4H), 8.19 (dd, J=8.8, 5.5 Hz, 2H), 7.64 (dd, J=9.5, 2.6 Hz, 2H), 7.55-7.43 (m, 2H), 4.56 (s, 4H). MS (ES+) m/z 431.1 (M+1).

### Example 2.5

### Synthesis of (4-{[2-({[amino(imino)methyl]thio}methyl)-4-fluorophenyl]thio}-3-thienyl)methyl imidothiocarbamate dihydrochloride

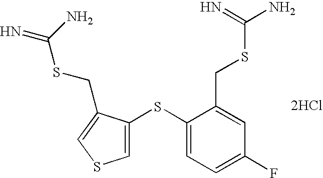

Following the procedure as described in Example 2, making non-critical variations using 3-(chloromethyl)-4-(2-(chloromethyl)-4-fluorophenylthio)thiophene to replace bis(2-(bromomethyl)-4-fluorophenyl)sulfane, (4-{[2-({[amino(imino)methyl]thio}methyl)-4-fluorophenyl]thio}-3-thienyl)methyl imidothiocarbamate dihydrochloride dihydrochloride was obtained as a colorless solid in 98% yield: ¹H NMR (300 MHz, DMSO-d₆) δ 9.53-9.14 (m, 8H), 7.83 (d, J=3.2 Hz, 1H), 7.78 (d, J=3.2 Hz, 1H), 7.47 (dd, J=9.5, 2.8 Hz, 1H), 7.24-7.10 (m, 1H), 6.99 (dd, J=8.5, 5.6 Hz, 1H), 4.67 (s, 2H), 4.37 (s, 2H); ¹³C NMR (75 MHz, DMSO-d₆) δ 169.6, 169.4, 161.3 (d, J_{C-F}=245.2 Hz), 136.5 (d, J_{C-F}=8.2 Hz), 135.8, 133.3, 132.7 (d, J_{C-F}=8.5 Hz), 132.0 (d, J_{C-F}=3.0 Hz), 128.4, 127.9, 118.2 (d, J_{C-F}=23.0 Hz), 117.2 (d, J_{C-F}=23.0 Hz), 33.4, 28.8; MS (ES+) m/z 367.1 (M+1).

### Example 2.6

### Synthesis of (2-{[2-({[amino(imino)methyl]thio}methyl)-4-fluorophenyl]thio}-3-thienyl)methyl imidothiocarbamate dihydrochloride

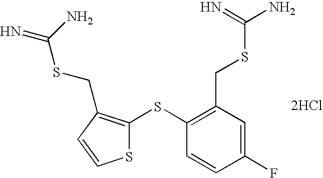

Following the procedure as described in Example 2, making non-critical variations using 3-(chloromethyl)-2-(2-(chloromethyl)-4-fluorophenylthio)thiophene to replace bis(2-(bromomethyl)-4-fluorophenyl)sulfane, (2-{[2-({[amino(imino)methyl]thio}methyl)-4-fluorophenyl]thio}-3-thienyl)methyl imidothiocarbamate dihydrochloride was obtained as a colorless solid in 94% yield: ¹H NMR (300 MHz, DMSO-d₆) δ 9.70-9.25 (m, 8H), 7.82 (d, J=5.4 Hz, 1H), 7.52 (dd, J=9.5, 2.7 Hz, 1H), 7.28 (d, J=5.4 Hz, 1H), 7.23-7.08 (m, 2H), 7.00 (dd, J=8.8, 5.4 Hz, 1H), 4.76 (s, 2H), 4.59 (s, 2H); ¹³C NMR (75 MHz, DMSO-d₆) δ 169.5, 169.2, 161.3 (d, J_{C-F}=245.8 Hz), 146.0, 141.2, 136.3 (d, J_{C-F}=8.2 Hz), 132.8 (d, J_{C-F}=3.1 Hz), 132.6, 132.2 (d, J_{C-F}=8.3 Hz), 130.2, 128.9, 118.2 (d, J_{C-F}=23.1 Hz), 117.3 (d, J_{C-F}=22.0 Hz), 33.2, 28.8; MS (ES+) m/z 387.1 (M+1).

### Example 2.7

### Synthesis of 2-{[2-({[amino(imino)methyl]thio}methyl)-4-fluorophenyl]thio}-4-(methylsulfonyl)benzyl imidothiocarbamate dihydrochloride

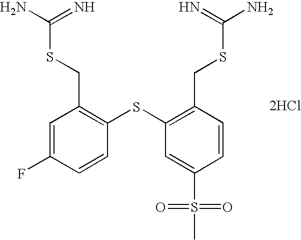

Following the procedure as described in Example 2, making non-critical variations using (2-(chloromethyl)-4-fluorophenyl)(2-(chloromethyl)-5-(methylsulfonyl)phenyl)sulfane to replace bis(2-(bromomethyl)-4-fluorophenyl)sulfane, 2-{[2-({[amino(imino)methyl]thio}methyl)-4-fluorophenyl]thio}-4-(methylsulfonyl)benzyl imidothiocarbamate dihydrochloride was obtained as a colorless solid in 92% yield: ¹H NMR (300 MHz, DMSO-d₆) δ 9.50 (br s, 2H), 9.40 (br s, 2H), 9.36 (br s, 2H), 9.28 (br s, 2H), 7.83-7.80 (m, 2H), 7.64 (dd, J=9.5, 2.8 Hz, 1H), 7.46 (dd, J=8.7, 5.7 Hz, 1H), 7.33 (dd, J=8.7, 2.8 Hz, 1H), 7.30-7.27 (m, 1H), 4.77 (s, 2H), 4.65 (s, 2H), 3.14 (s, 3H); ¹³C NMR (75 MHz, DMSO-d₆) δ 169.1, 162.7 (d, J_{C-F}=248.2 Hz), 141.9, 140.8, 140.7, 138.2 (d, J_{C-F}=8.7 Hz), 139.8, 138.5, 132.2, 128.1, 126.2, 118.9 (d, J_{C-F}=23.6 Hz), 117.8 (d, J_{C-F}=21.5 Hz), 43.8, 33.6, 33.2; MS (ES+) m/z 459.1 (M+1).

### Example 2.8

### Synthesis of 2-{[2-({[amino(imino)methyl]thio}methyl)-4-chlorophenyl]thio}-5-cyanobenzyl imidothiocarbamate dihydrobromide

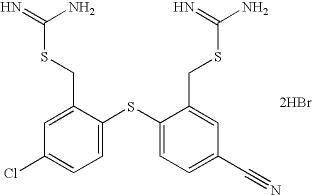

Following the procedure as described in Example 2, making non-critical variations using 3-(bromomethyl)-4-(2-(bromomethyl)-4-chlorophenylthio)benzonitrile to replace bis(2-(bromomethyl)-4-fluorophenyl)sulfane, 2-{[2-({[amino(imino)methyl]thio}methyl)-4-chlorophenyl]thio}-5-cyanobenzyl imidothiocarbamate dihydrobromide was obtained as a colorless solid in 94% yield: ¹H NMR (300 MHz, DMSO-d₆) δ 9.41-8.93 (m, 8H), 7.98 (d, J=1.8 Hz, 1H), 7.80 (d, J=2.3 Hz, 1H), 7.68 (dd, J=8.3, 1.8 Hz, 1H), 7.57-7.43 (m, 2H), 6.88 (d, J=8.3 Hz, 1H), 4.66 (s, 2H), 4.54 (s, 2H); ¹³C NMR (75 MHz, DMSO-d₆) δ 168.8, 143.9, 140.8, 138.4, 135.4, 134.6, 134.2, 133.2, 131.6, 130.7, 130.3, 130.1, 118.5, 109.5, 33.5, 33.1; MS (ES+) m/z 423.1 (M+1).

### Example 2.9

### Synthesis of 2-[2-({[amino(imino)methyl]thio}methyl)-5-chlorophenoxy]-4-nitrobenzyl imidothiocarbamate dihydrobromide

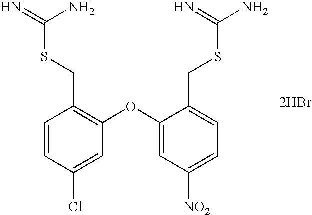

Following the procedure as described in Example 2, making non-critical variations using 1-(bromomethyl)-2-(2-(bromomethyl)-5-chlorophenoxy)-4-nitrobenzene to replace bis(2-(bromomethyl)-4-fluorophenyl)sulfane, 2-[2-({[amino(imino)methyl]thio}methyl)-5-chlorophenoxy]-4-nitrobenzyl imidothiocarbamate dihydrobromide was obtained as a colorless solid in 65% yield: mp>220° C.; ¹H NMR (300 MHz, CD₃OD) δ 8.11 (dd, J=8.4, 1.8 Hz, 1H), 7.85 (d, J=7.8 Hz, 1H), 7.66-7.60 (m, 2H), 7.36 (dd, J=8.2, 1.6 Hz, 1H), 7.11 (dd, J=1.7 Hz, 1H), 4.71 (s, 2H), 4.58 (s, 2H); ¹³C NMR (75 MHz, CD₃OD) δ 168.8, 168.7, 153.6, 152.7, 147.5, 134.2, 131.1, 130.7, 130.5, 124.0, 123.2, 117.8, 117.3, 110.4, 28.3. 28.2; MS (ES+) m/z 426.2 (M+1) and 428.1 (M+1).

### Example 2.10

### Synthesis of 2-[2-(2-carbamimidoylsulfanylmethyl-3-chlorophenoxy)-5-fluorobenzyl]isothiourea dihydrobromide

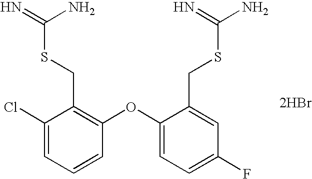

Following the procedure as described in Example 2, making non-critical variations using 1-(bromomethyl)-2-(2-(bromomethyl)-4-fluorophenoxy)-4-chlorobenzene to replace bis(2-(bromomethyl)-4-fluorophenyl)sulfane, 2-[2-(2-carbamimidoylsulfanylmethyl-3-chlorophenoxy)-5-fluorobenzyl]isothiourea dihydrobromide was obtained as a colorless solid in 90% yield: mp 204-206° C.; ¹H NMR (300 MHz, CD₃OD) δ 7.42-7.24 (m, 3H), 7.24-7.15 (m, 1H), 7.00 (dd, J=9.1, 4.6 Hz, 1H), 6.78 (dd, J=7.8, 1.5 Hz, 1H), 4.76 (s, 2H), 4.53 (s, 2H); ¹³C NMR (75 MHz, CD₃OD) δ 169.5, 168.9, 159.3, 156.0, 148.6 (d, J_{C-F}=2.9 Hz), 134.1, 129.3, 126.1 (d, J_{C-F}=8.1 Hz), 123.2, 120.9, 119.5 (d, J_{C-F}=8.7 Hz), 116.1 (d, J_{C-F}=24.5 Hz), 115.4 (d, J_{C-F}=23.6 Hz), 114.1, 28.6, 26.7; MS (ES+) m/z 399.2 (M+1) 401.2 (M+1).

### Example 2.11

### Synthesis of 2-[2-(2-carbamimidoylsulfanylmethyl-4-chlorophenoxy)-5-fluorobenzyl]isothiourea dihydrobromide

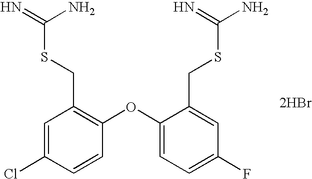

Following the procedure as described in Example 2, making non-critical variations using 1-(bromomethyl)-2-(2-(bromomethyl)-4-fluorophenoxy)-4-chlorobenzene to replace bis(2-(bromomethyl)-4-fluorophenyl)sulfane, 2-[2-(2-carbamimidoylsulfanylmethyl-4-chlorophenoxy)-5-fluorobenzyl]isothiourea dihydrobromide was obtained as a colorless solid in 90% yield: mp>230° C.; ¹H NMR (300 MHz, CD₃OD) δ 7.59 (d, J=2.5 Hz, 1H), 7.40-7.33 (m, 2H), 7.17 (td, J=8.8, 3.1 Hz, 1H), 6.98 (dd, J=9.0, 4.6 Hz, 1H), 6.82 (d, J=8.8 Hz, 1H), 4.57 (s, 2H), 4.53 (s, 2H); ¹³C NMR (75 MHz, CD₃OD) δ 169.0, 168.9, 159.1, 155.9, 152.5, 148.8 (d, J_{C-F}=2.7 Hz), 129.2, 128.6, 127.1, 126.0 (d, J_{C-F}=8.1 Hz), 125.1, 119.1 (d, J_{C-F}=8.6 Hz), 117.1, 116.0 (d, J_{C-F}=24.5 Hz), 115.3 (d, J_{C-F}=23.6 Hz), 28.5 (m, 2C); MS (ES+) m/z 399.2 (M+1) and 401.2 (M+1).

### Example 2.12

### Synthesis of 2-[2-(2-carbamimidoylsulfanylmethyl-5-fluorophenoxy)-5-fluorobenzyl]isothiourea dihydrobromide

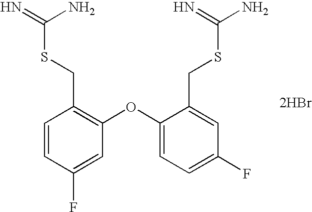

Following the procedure as described in Example 2, making non-critical variations using 1-(bromomethyl)-2-(2-(bromomethyl)-4-fluorophenoxy)-4-fluorobenzene to replace bis(2-(bromomethyl)-4-fluorophenyl)sulfane, 2-[2-(2-carbamimidoylsulfanylmethyl-5-fluorophenoxy)-5-fluorobenzyl]isothiourea dihydrobromide was obtained as a colorless solid in 87% yield: mp>240° C.; ¹H NMR (300 MHz, CD₃OD) δ 7.57 (dd, J=8.6, 6.3 Hz, 1H), 7.39 (dd, J=8.7, 3.0 Hz, 1H), 7.26-7.16 (m, 1H), 7.05 (dd, J=9.0, 4.6 Hz, 1H), 7.00-6.90 (m, 1H), 6.57 (dd, J=9.9, 2.5 Hz, 1H), 4.58 (s, 2H), 4.51 (s, 2H); ¹³C NMR (75 MHz, DMSO-d₆) δ 169.6, 169.3, 164.6, 161.3, 160.3, 157.1, 156.5 (d, J_{C-F}=10.1 Hz), 150.0 (d, J_{C-F}=2.6 Hz), 133.1 (d, J_{C-F}=9.8 Hz), 129.1 (d, J_{C-F}=8.3 Hz), 121.6, 118.20 (d, J_{C-F}=24.7 Hz), 117.4 (d, J_{C-F}=23.25 Hz), 110.95 (d, J_{C-F}=21.3 Hz), 105.0 (d, J_{C-F}=25.8 Hz), 29.89; MS (ES+) m/z 383.4 (M+1).

### Example 2.13

### Synthesis of 2-[2-(2-carbamimidoylsulfanylmethyl-4-fluorophenoxy)-5-fluorobenzyl]isothiourea dihydrobromide

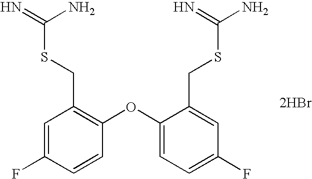

Following the procedure as described in Example 2, making non-critical variations using 4,4′-oxybis(3-(bromomethyl)-1-fluorobenzene) to replace bis(2-(bromomethyl)-4-fluorophenyl)sulfane, 2-[2-(2-carbamimidoylsulfanylmethyl-4-fluorophenoxy)-5-fluorobenzyl]isothiourea dihydrobromide was obtained as a colorless solid in 84% yield: mp>230° C.; ¹H NMR (300 MHz, DMSO-d₆) δ 9.28-8.96 (m, 8H), 7.46 (m, 2H), 7.23-7.12 (m, 2H), 6.83 (dd, J=9.1, 4.7 Hz, 2H), 4.52 (s, 4H); ¹³C NMR (75 MHz, DMSO-d₆) δ 169.3, 156.6, 151.2 (d, J_{C-F}=2.3 Hz), 128.1 (d, J_{C-F}=8.3 Hz), 120.1 (d, J_{C-F}=8.3 Hz), 118.1 (d, J_{C-F}=24.0 Hz), 117.2 (d, J_{C-F}=23.2 Hz), 29.95; MS (ES+) m/z 383.4 (M+1).

### Example 2.14

### Synthesis of 2-[2-({[amino(imino)methyl]thio}methyl)-4-fluorophenoxy]-5-nitrobenzyl imidothiocarbamate dihydrobromide

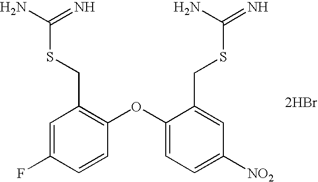

Following the procedure as described in Example 2, making non-critical variations using 2-(bromomethyl)-1-(2-(bromomethyl)-4-fluorophenoxy)-4-nitrobenzene to replace bis(2-(bromomethyl)-4-fluorophenyl)sulfane, 2-[2-({[amino(imino)methyl]thio}methyl)-4-fluorophenoxy]-5-nitrobenzyl imidothiocarbamate dihydrobromide was obtained as a colorless solid in 88% yield: mp>220° C.; ¹H NMR (300 MHz, CD₃OD) δ 8.49 (d, J=2.7 Hz, 1H), 8.24 (dd, J=9.0, 2.7 Hz, 1H), 7.44 (dd, J=9.0, 3.0 Hz, 1H), 7.31-7.14 (m, 2H), 6.91 (d, J=9.3 Hz, 1H), 4.74 (s, 2H), 4.52 (s, 2H); ¹³C NMR (75 MHz, CD₃OD) δ 171.7 (d, J_{C-F}=11.6 Hz), 162.9, 162.0, 159.6, 150.2 (d, J_{C-F}=2.9 Hz), 144.4, 130.1 (d, J_{C-F}=8.2 Hz), 127.7, 127.2, 126.8, 124.0 (d, J_{C-F}=8.9 Hz), 119.2 (d, J_{C-F}=24.5 Hz), 118.7 (d, J_{C-F}=23.6 Hz), 171.1, 31.4, 31.3; MS (ES+) m/z 410.2 (M+1).

### Example 2.15

### Synthesis of 2-[2-(2-carbamimidoylsulfanylmethylphenoxy)benzyl]isothiourea dihydrobromide

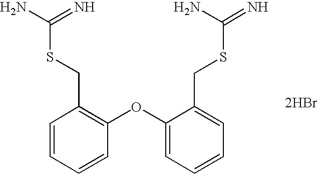

Following the procedure as described in Example 2, making non-critical variations using 2,2′-oxybis((bromomethyl)benzene to replace bis(2-(bromomethyl)-4-fluorophenyl)sulfane, 2-[2-(2-carbamimidoylsulfanylmethyl-phenoxy)benzyl]isothiourea dihydrobromide was obtained as a colorless solid in 39% yield: mp>220° C.; ¹H NMR (300 MHz, CD₃OD) δ 7.56 (dd, J=7.5, 1.5 Hz, 2H), 7.43-7.34 (m, 2H), 7.26-7.16 (m, 2H), 6.88 (d, J=8.4 Hz, 2H), 4.57 (s, 4H); ¹³C NMR (75 MHz, CD₃OD) δ 172.4, 156.2, 132.6, 131.7, 126.1, 125.6, 119.5, 31.8; MS (ES+) m/z 347.2 (M+1).

### Example 2.16

### Synthesis of 2-[2-({[amino(imino)methyl]thio}methyl)-4-chlorophenoxy]benzyl imidothiocarbamate dihydrobromide

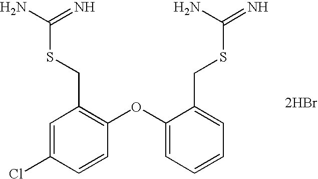

Following the procedure as described in Example 2, making non-critical variations using 2-(bromomethyl)-1-(2-(bromomethyl)phenoxy)-4-chlorobenzene to replace bis(2-(bromomethyl)-4-fluorophenyl)sulfane, 2-[2-({[amino(imino)methyl]thio}methyl)-4-chlorophenoxy]benzyl imidothiocarbamate dihydrobromide was obtained as a colorless solid in 88% yield: mp>220° C.; ¹H NMR (300 MHz, CD₃OD) δ 7.62-7.53 (m, 2H), 7.45-7.34 (m, 2H), 7.23 (dd, J=7.5, 7.5 Hz, 1H), 6.92 (d, J=8.4 Hz, 1H), 6.85 (d, J=9.0 Hz, 1H), 4.56 (s, 4H); ¹³C NMR (75 MHz, CD₃OD) δ 172.3, 172.0, 155.9, 155.2, 132.8, 132.1, 131.9, 131.5, 130.1, 128.3, 126.4, 126.0, 120.7, 119.9, 31.8, 31.4; MS (ES+) m/z 381.2 (M+1).

### Example 2.17

### Synthesis of 2-[2-({[amino(imino)methyl]thio}methyl)-3-chlorophenoxy]benzyl imidothiocarbamate dihydrobromide

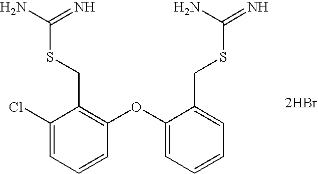

Following the procedure as described in Example 2, making non-critical variations using 2-(bromomethyl)-1-(2-(bromomethyl)phenoxy)-3-chlorobenzene to replace bis(2-(bromomethyl)-4-fluorophenyl)sulfane, 2-[2-({[amino(imino)methyl]thio}methyl)-3-chlorophenoxy]benzyl imidothiocarbamate dihydrobromide was obtained as a colorless solid in 93% yield: mp 150-152° C.; ¹H NMR (300 MHz, CD₃OD) δ 7.60 (dd, J=7.5, 1.5 Hz, 1H), 7.46-7.22 (m, 4H), 6.94 (d, J=8.1 Hz, 1H), 6.82 (dd, J=7.5, 1.5 Hz, 1H), 4.76 (s, 2H), 4.58 (s, 2H); ¹³C NMR (75 MHz, CD₃OD) δ 172.5, 172.2, 157.7, 155.7, 137.0, 132.9, 132.2, 131.9, 126.5, 126.3, 126.2, 124.2, 120.2, 117.6, 31.8, 29.6; MS (ES+) m/z 381.2 (M+1).

### Example 2.18

### Synthesis of 2-[2-({[amino(imino)methyl]thio}methyl)-5-chlorophenoxy]benzyl imidothiocarbamate dihydrobromide

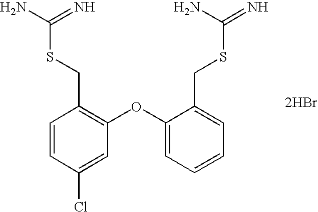

Following the procedure as described in Example 2, making non-critical variations using 1-(bromomethyl)-2-(2-(bromomethyl)phenoxy)-4-chlorobenzene to replace bis(2-(bromomethyl)-4-fluorophenyl)sulfane, 2-[2-({[amino(imino)methyl]thio}methyl)-5-chlorophenoxy]benzyl imidothiocarbamate dihydrobromide was obtained as a colorless solid in 74% yield: mp>220° C.; ¹H NMR (300 MHz, CD₃OD) δ 7.63-7.53 (m, 2H), 7.49-7.40 (m, 1H), 7.28 (dd, J=7.8, 7.8 Hz, 1H), 7.21 (dd, J=8.1, 1.5 Hz, 1H), 6.98 (d, J=8.1 Hz, 1H), 6.81 (d, J=1.5 Hz, 1H), 4.59 (s, 2H), 4.56 (s, 2H); ¹³C NMR (75 MHz, CD₃OD) δ 172.3, 172.2, 157.2, 155.4, 136.8, 133.7, 132.9, 132.0, 126.7, 126.5, 125.4, 124.9, 120.3, 119.0, 31.7, 31.4; MS (ES+) m/z 381.2 (M+1).

### Example 2.19

### Synthesis of 2-[2-({[amino(imino)methyl]thio}methyl)-5-chlorophenoxy]-5-nitrobenzyl imidothiocarbamate dihydrobromide

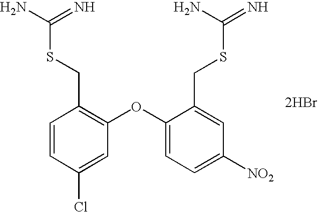

Following the procedure as described in Example 2, making non-critical variations using 1-(bromomethyl)-2-(2-(bromomethyl)-4-nitrophenoxy)-4-chlorobenzene to replace bis(2-(bromomethyl)-4-fluorophenyl)sulfane, 2-[2-({[amino(imino)methyl]thio}methyl)-5-chlorophenoxy]-5-nitrobenzyl imidothiocarbamate dihydrobromide was obtained as a colorless solid in 84% yield: mp>220° C.; ¹H NMR (300 MHz, CD₃OD) δ 8.51 (d, J=2.7 Hz, 1H), 8.27 (dd, J=9.0, 2.7 Hz, 1H), 7.65 (d, J=8.4 Hz, 1H), 7.37 (dd, J=8.4, 2.1 Hz, 1H), 7.19 (d, J=2.1 Hz, 1H), 7.01 (d, J=9.0 Hz, 1H), 4.74 (s, 2H), 4.56 (s, 2H); ¹³C NMR (75 MHz, CD₃OD) δ 171.7, 161.2, 155.1, 144.9, 137.1, 134.1, 127.8, 127.5, 127.4, 127.3, 126.6, 121.8, 118.1, 31.4, 31.2; MS (ES+) m/z 426.2 (M+1), 428.2 (M+1).

### Example 2.20

### Synthesis of 2-[2-({[amino(imino)methyl]thio}methyl)-4-chlorophenoxy]-5-nitrobenzyl imidothiocarbamate dihydrobromide

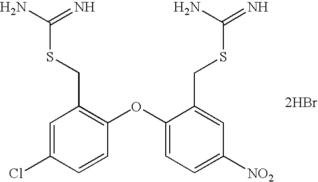

Following the procedure as described in Example 2, making non-critical variations using 2-(bromomethyl)-1-(2-(bromomethyl)-4-chlorophenoxy)-4-nitrobenzene to replace bis(2-(bromomethyl)-4-fluorophenyl)sulfane, 2-[2-({[amino(imino)methyl]thio}methyl)-4-chlorophenoxy]-5-nitrobenzyl imidothiocarbamate dihydrobromide was obtained as a colorless solid in 91% yield: mp>220° C.; ¹H NMR (300 MHz, CD₃OD) δ 8.51 (d, J=2.7 Hz, 1H), 8.25 (dd, J=9.0, 2.7 Hz, 1H), 7.69 (d, J=2.4 Hz, 1H), 7.50 (dd, J=8.7, 2.4 Hz, 1H), 7.14 (d, J=8.7 Hz, 1H), 6.98 (d, J=9.0 Hz, 1H), 4.74 (s, 2H), 4.55 (s, 2H); ¹³C NMR (75 MHz, CD₃OD) δ 171.7, 171.6, 161.5, 153.1, 144.7, 132.6, 132.2, 132.0, 129.9, 127.8, 127.3, 123.3, 117.9, 31.4, 31.3; MS (ES+) m/z 426.2 (M+1), 428.2 (M+1).

### Example 2.21

### Synthesis of 2-[2-({[amino(imino)methyl]thio}methyl)-4-fluorophenoxy]benzyl imidothiocarbamate dihydrobromide

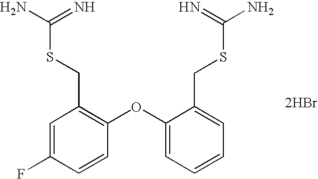

Following the procedure as described in Example 2, making non-critical variations using 2-(bromomethyl)-1-(2-(bromomethyl)phenoxy)-4-fluorobenzene to replace bis(2-(bromomethyl)-4-fluorophenyl)sulfane, 2-[2-({[amino(imino)methyl]thio}methyl)-4-fluorophenoxy]benzyl imidothiocarbamate dihydrobromide was obtained as a colorless solid in 87% yield: mp>220° C.; ¹H NMR (300 MHz, DMSO-d₆) δ 9.40-8.90 (br s, 8H), 7.57 (dd, J=7.5, 1.2 Hz, 1H), 7.49 (dd, J=9.0, 3.0 Hz, 1H), 7.42-7.15 (m, 3H), 6.90 (dd, J=9.0, 4.5 Hz, 1H), 6.80 (d, J=7.5 Hz, 1H), 4.56 (s, 2H), 4.52 (s, 2H); ¹³C NMR (75 MHz, DMSO-d₆) δ 169.6, 169.3, 159.8, 156.7, 155.2, 150.9 (d, J_{C-F}=12.7 Hz), 131.7, 130.7, 128.4 (J_{C-F}=8.1 Hz), 125.6, 124.4, 120.6 (J_{C-F}=8.6 Hz), 117.9, 117.2 (J_{C-F}=23.6 Hz), 30.3, 29.9; MS (ES+) m/z 365.3 (M+1).

### Example 2.22

### Synthesis of (6,6′-dimethylbiphenyl-2,2′-diyl)bis(methylene)dicarbamimidothioate dihydrobromide

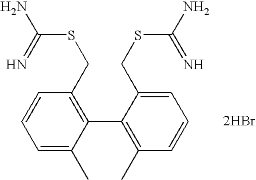

Following the procedure as described in Example 2, making non-critical variations using 2,2′-bis(bromomethyl)-6,6′-dimethylbiphenyl (prepared according to Mislow, et al., J. Am. Chem. Soc. 1964; 86(9):1710-1733) to replace bis(2-(bromomethyl)-4-fluorophenyl)sulfane, (6,6′-dimethylbiphenyl-2,2′-diyl)bis(methylene)dicarbamimidothioate dihydrobromide was obtained as a colorless solid in 93% yield: mp 146-150° C. (hexanes); ¹H NMR (300 MHz, DMSO-d₆) δ 9.07 (br s, 4H), 8.98 (br s, 4H), 7.49-7.44 (m, 2H), 7.42-7.34 (m, 4H), 4.16 (d, J=12.2 Hz, 2H), 3.88 (d, J=12.2 Hz, 2H), 1.90 (s, 6H); ¹³C NMR (75 MHz, DMSO-d₆) δ 169.1, 137.7, 136.8, 131.6, 130.5, 128.5, 128.2, 33.5, 20.0; MS (ES+) m/z 359.2 (M+1).

### Example 2.23

### Synthesis of biphenyl-2,2′-6,6′-tetrayltetrakis(methylene)tetracarbamimidothioate tetrahydrobromide

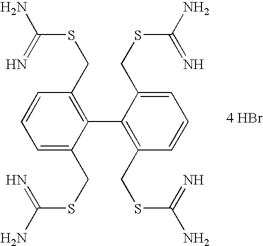

Following the procedure as described in Example 2, making non-critical variations using 2,2′,6,6′-tetrakis(bromomethyl)biphenyl (prepared according to Mislow, et al., J. Am. Chem. Soc. 1964; 86(9):1710-1733) to replace bis(2-(bromomethyl)-4-fluorophenyl)sulfane, biphenyl-2,2′-6,6′-tetrayltetrakis(methylene)tetracarbamimidothioate tetrahydrobromide was obtained as a fine, colorless powder in 53% yield: mp 233-235° C. (ethanol/acetonitrile); ¹H NMR (300 MHz, DMSO-d₆) δ 9.09 (br s, 16H), 7.69-7.55 (m, 6H), 4.06 (s, 8H); ¹³C NMR (75 MHz, DMSO-d₆) δ 168.9, 135.9, 133.5, 130.6, 129.9, 33.4; MS (ES+) m/z 507.2 (M+1).

### Example 2.24

### Synthesis of dimethyl 6,6′-bis(carbamimidoylthiomethyl)biphenyl-2,2′-dicarboxylate dihydrobromide

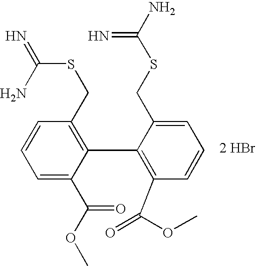

Following the procedure as described in Example 2, making non-critical variations using 6,6′-bis(bromomethyl)biphenyl-2,2′-dicarboxylate (prepared according to Mislow, et al., J. Am. Chem. Soc. 1964; 86(9):1710-1733) to replace bis(2-(bromomethyl)-4-fluorophenyl)sulfane, dimethyl 6,6′-bis(carbamimidoylthiomethyl)biphenyl-2,2′-dicarboxylate dihydrobromide was obtained as a colorless solid in 93% yield: mp 222-225° C. (ethanol/t-butyl methyl ether); ¹H NMR (300 MHz, DMSO-d₆) δ 8.93 (br s, 8H), 8.00 (dd, J=7.8, 0.9 Hz, 2H), 7.86 (dd, J=7.8, 0.9 Hz, 2H), 7.61 (dd, J=7.8, 7.8 Hz, 2H), 4.16 (d, J=12.2 Hz, 2H), 3.82 (d, J=12.2 Hz, 2H), 3.52 (s, 6H); ¹³C NMR (75 MHz, DMSO-d₆) δ 169.1, 165.8, 139.4, 134.5, 132.6, 130.2, 129.9, 128.6, 51.9, 33.2; MS (ES+) m/z 447.2 (M+1).

### Example 2.25

### Synthesis of 2-(6-(amidinothiomethyl)phenyl)carbonyl-1-(amidinothiomethyl)benzene dihydrobromide

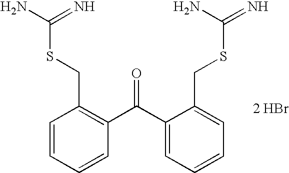

Following the procedure as described in Example 2, making non-critical variations using bis(2-(bromomethyl)phenyl)methanone to replace bis(2-(bromomethyl)-4-fluorophenyl)sulfane, 2-(6-(amidinothiomethyl)phenyl)carbonyl-1-(amidinothiomethyl)benzene dihydrobromide was obtained as a colorless solid in 40% yield: mp 239-240° C. (dec.); ¹H NMR (300 MHz, DMSO-d₆) δ 9.21 (br s, 4H), 9.04 (br s, 4H), 7.63 (m, 4H), 7.45 (m, 2H), 7.25 (d, J=6.0 Hz, 2H), 4.68 (s, 4H); ¹³C NMR (75 MHz, DMSO-d₆) δ 199.1, 169.5, 137.5, 135.9, 133.1, 132.3, 131.9, 128.7, 32; MS (ES+) m/z 359.2 (M+1).

### Example 2.26

### Synthesis of 2-(6-(amidinothiomethyl)phenyl)carbonyl-1-(amidinothiomethyl)-5-fluorobenzene dihydrobromide

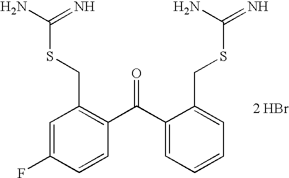

Following the procedure as described in Example 2, making non-critical variations using (2-(bromomethyl)-4-fluorophenyl)(2-(bromomethyl)phenyl)methanone to replace bis(2-(bromomethyl)-4-fluorophenyl)sulfane, 2-(6-(amidinothiomethyl)phenyl)carbonyl-1-(amidinothiomethyl)-5-fluorobenzene dihydrobromide was obtained as a colorless solid in 37% yield: mp>250° C.; ¹H NMR (300 MHz, DMSO-d₆) δ 9.35-9.85 (m, 8H), 7.72-7.59 (m, 3H), 7.53-7.42 (m, 1H), 7.44-7.28 (m, 3H), 4.71 (s, 2H), 4.66 (s, 2H); ¹³C NMR (75 MHz, DMSO-d₆) δ 197.9, 169.5, 169.3, 139.9 (d, J_{C-F}=8.5 Hz), 137.5, 135.8, 135.5 (d, J_{C-F}=9.5 Hz), 133.9, (d, J_{C-F}=3.0 Hz), 133.0, 132.1, 131.8, 128.7, 119.0 (d, J_{C-F}=23.1 Hz), 115.5 (d, J_{C-F}=21.4 Hz), 32.8, 32.5; MS (ES+) m/z 377.2 (M+1).

### Example 2.27

### Synthesis of 2-(6-(amidinothiomethyl)phenyl)carbonyl-1-(amidinothiomethyl)-4-fluorobenzene dihydrobromide

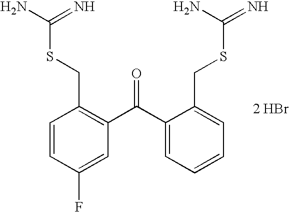

Following the procedure as described in Example 2, making non-critical variations using (2-(bromomethyl)-5-fluorophenyl)(2-(bromomethyl)phenyl)methanone to replace bis(2-(bromomethyl)-4-fluorophenyl)sulfane, 2-(6-(amidinothiomethyl)phenyl)carbonyl-1-(amidinothiomethyl)-4-fluorobenzene dihydrobromide was obtained as a colorless solid in 58% yield: mp>250° C.; ¹H NMR (300 MHz, DMSO-d₆) δ 9.31-8.91 (m, 8H), 7.75-7.69 (m, 1H), 7.68-7.65 (m, 2H), 7.55-7.42 (m, 2H), 7.37-7.28 (m, 1H), 7.08 (dd, J=8.9, 2.7 Hz, 1H), 4.65 (s, 2H), 4.58 (s, 2H); ¹³C NMR (300 MHz, DMSO-d₆) δ 197.7, 169.4, 169.3, 164.1, 161.2 (d, J_{C-F}=248.0 Hz), 139.7 (d, J_{C-F}=6.2 Hz), 136.5 (d, J_{C-F}=3.9 Hz), 134.2, 134.1, 133.5, 132.7, 131.7 (d, J_{C-F}=3.4 Hz), 128.8, 119.8 (d, J_{C-F}=21.1 Hz), 118.6 (d, J_{C-F}=23.1 Hz), 33.0, 32.1; MS (ES+) m/z 377.2 (M+1).

### Example 2.28

### Synthesis of 2-(6-(amidinothiomethyl)phenyl)carbonyl-1-(amidinothiomethyl)-4-chlorobenzene dihydrobromide dihydrobromide

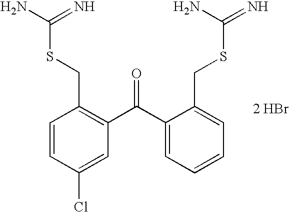

Following the procedure as described in Example 2, making non-critical variations using (2-(bromomethyl)-5-chlorophenyl)(2-(bromomethyl)phenyl)methanone to replace bis(2-(bromomethyl)-4-fluorophenyl)sulfane, 2-(6-(amidinothiomethyl)phenyl)carbonyl-1-(amidinothiomethyl)-4-chlorobenzene dihydrobromide was obtained as a colorless solid in 68% yield: mp>250° C.; ¹H NMR (300 MHz, DMSO-d₆) δ 9.36-9.04 (m, 8H), 7.80-7.67 (m, 4H), 7.55-7.46 (m, 1H), 7.38-7.35 (m, 1H), 7.31-7.28 (m, 1H), 4.71 (s, 2H), 4.65 (s, 2H); ¹³C NMR (75 MHz, DMSO-d₆) δ 97.6, 169.5, 169.2, 139.5, 136.5, 136.4, 134.6, 133.6, 133.5, 133.1, 132.7, 132.6, 131.9, 131.2, 128.8, 33.0, 32.2; MS (ES+) m/z 393.1 (M+1).

### Example 2.29

### Synthesis of 2-(6-(amidinothiomethyl)phenyl)carbonyl-1-(amidinothiomethyl)-5-chlorobenzene dihydrobromide

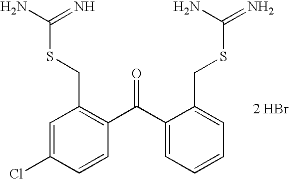

Following the procedure as described in Example 2, making non-critical variations using (2-(bromomethyl)-4-chlorophenyl)(2-(bromomethyl)phenyl)methanone to replace bis(2-(bromomethyl)-4-fluorophenyl)sulfane, 2-(6-(amidinothiomethyl)phenyl)carbonyl-1-(amidinothiomethyl)-5-chlorobenzene dihydrobromide was obtained as a colorless solid in 16% yield: mp>250° C.; ¹H NMR (300 MHz, DMSO-d₆) δ 9.24-8.92 (m, 8H), 7.80-7.78 (m, 1H), 7.66-7.62 (m, 2H), 7.60-7.51 (m, 1H), 7.49-7.41 (m, 1H), 7.30-7.29 (m, 1H), 7.29-7.28 (m, 1H), 4.62 (s, 4H); (75 MHz, DMSO-d₆) δ 198.1, 169.4, 169.2, 138.6, 137.4, 137.1, 136.1, 136.0, 134.2, 133.2, 132.3, 131.8, 131.4, 128.7, 128.6, 32.8, 32.4; MS (ES+) m/z 393.2 (M+1).

### Example 2.30

### Synthesis of 2-[2-({[amino(imino)methyl]thio}methyl)-5-chlorophenoxy]-5-fluorobenzyl imidothiocarbamate dihydrobromide

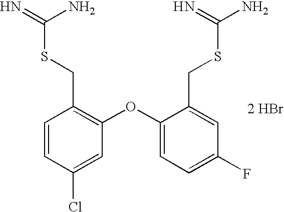

Following the procedure as described in Example 2, making non-critical variations using 1-(bromomethyl)-2-(2-(bromomethyl)-4-fluorophenoxy)-4-chlorobenzene to replace bis(2-(bromomethyl)-4-fluorophenyl)sulfane, 2-(6-(amidinothiomethyl)phenyl)carbonyl-1-(amidinothiomethyl)-5-chlorobenzene dihydrobromide was obtained as a colorless solid in 93% yield: mp>220° C.; ¹H NMR (300 MHz, CD₃OD) δ 7.39-7.25 (m, 3H), 7.20-7.10 (m, 1H), 6.98 (dd, J=8.9, 4.5 Hz, 1H), 6.75 (d, J=7.7 Hz, 1H), 4.73 (s, 2H), 4.51 (s, 2H); ¹³C NMR (75 MHz, CD₃OD) δ 169.5, 168.9, 158.6 (d, J_{C-F}=92.5 Hz), 155.0, 148.6 (d, J_{C-F}=2.8 Hz), 134.1, 129.3, 126.1 (d, J_{C-F}=8.1 Hz), 123.2, 120.9, 119.5 (d, J_{C-F}=8.7 Hz), 116.1 (d, J_{C-F}=24.5 Hz), 115.4 (d, J_{C-F}=23.6 Hz), 114.1, 28.5, 26.6; MS (ES+) m/z 399.2 (M+1), 401.2 (M+1).

### Example 3

### Synthesis of 2-(6-((aminoamidino)thiomethyl)phenyl)thio-1-((aminoamidino)thiomethyl)benzene dihydrobromide

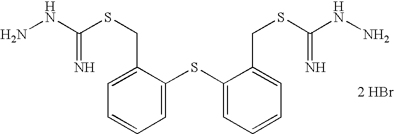

To a solution of bis(2-(bromomethyl)phenyl)sulfane (0.74 g, 2.00 mmol) in ethanol (24 mL) was added thiosemicarbazide (0.37 g, 4.08 mmol). The mixture was maintained at 80° C. for 1 h and cooled to ambient temperature. Ethanol was removed in vacuo. The precipitation was dried in vacuo at 80° C. for 16 hours to afford 2-(6-((aminoamidino)thiomethyl)phenyl)thio-1-((aminoamidino)thiomethyl)benzene dihydrobromide as a colorless solid in 80% yield (0.64 g): ¹H NMR (300 MHz, DMSO-d₆) δ 8.82 (m, 10H), 7.49-7.45 (m, 2H), 7.29-7.20 (m, 4H), 7.07-7.04 (m, 2H), 4.50 (s, 4H); ¹³C NMR (75 MHz, DMSO-d₆) δ 165.1, 136.6, 135.0, 133.1, 131.4, 130.0, 128.7, 33.7; MS (ES+) m/z 393.1 (M+1).

### Example 4

### Synthesis of 2,2′-thiobis(N-(diaminomethylene)benzamide)

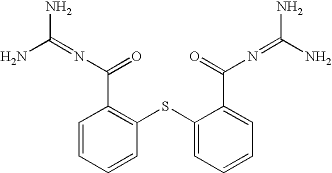

To an ice cold solution of 2,2′-thiodibenzoic acid (0.55 g, 2.00 mmol) and N-methylmorpholine (0.48 mL, 4.4 mmol) in N,N-dimethylformamide (10 mL) was added isobutylchloroformate (0.52 mL, 4.0 mmol). The reaction mixture was stirred at ambient temperature for 2 hours. To the above mixture was added guanidine (0.59 g, 10 mmol) in N,N-dimethylformamide (10 mL) of which was made from guanidinehydrochloride (0.95 g, 10 mmol) and sodium methoxide (0.54 g, 10.00 mmol) at ambient temperature. The solvent was evaporated and the residue was purified by column chromatography (methylene chloride/methanol=4/1) to afford 2,2′-thiobis(N-(diaminomethylene)benzamide) as a colorless solid in 8% yield (0.055 g): mp>220° C.; ¹H NMR (300 MHz, CD₃OD) δ 7.78-7.70 (m, 2H), 7.58-7.35 (m, 6H); ¹³C NMR (75 MHz, CD₃OD) δ 170.1, 156.8, 136.8, 136.2, 134.9, 134.0, 130.1, 129.2; MS (ES+) m/z 357.3 (M+1).

### Example 5

### Synthesis of 1-(2-(2-(guanidinomethyl)phenylthio)benzyl)guanidine (4-methylbenzenesulfonate)

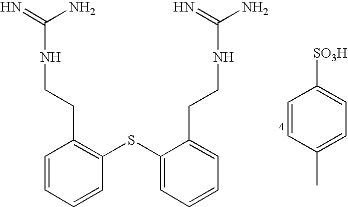

To a mixture of 1-(2-aminoethyl)-2-((6-(2-aminoethyl)phenyl)thio)benzene dihydrochloride (0.35 g, 1.0 mmol) and 1H-benzo[d][1,2,3]triazole-1-carboximidamide 4-methylbenzenesulfonate (prepared according to A. Katrizsky et al., Synth. Commun. 1995; 25(8):1173-1186) (0.67 g, 2.0 mmol) in N,N-dimethylformamide (5 mL) was added diisopropylethylamine (0.70 mL, 4.00 mmol) at ambient temperature. The reaction mixture was stirred at ambient temperature for 16 hours. The solvent was evaporated and the residue was purified by column chromatography (methylene chloride/methanol, 4/1) to afford 1-(2-(2-(guanidinomethyl)phenylthio)benzyl)guanidine (4-methylbenzenesulfonate) as a colorless solid in 2% yield (0.04 g): mp>200° C.; ¹H NMR (300 MHz, CD₃OD) δ 7.69 (d, J=8.1 Hz, 8H), 7.38-7.03 (m, 16H), 3.45 (t, J=6.6 Hz, 4H), 3.04 (t, J=6.6 Hz, 4H), 2.36 (s, 12H); ¹³C NMR (75 MHz, CD₃OD) δ 158.6, 143.3, 141.9, 139.9, 135.7, 133.2, 131.7, 129.9, 129.2, 129.1, 126.9, 42.7, 34.1, 21.3; MS (ES+) m/z 357.3 (M+1).

### Example 6

### Synthesis of 2-(6-(2-amidinoethyl)phenyl)thio-1-(2-amidinoethyl)benzene

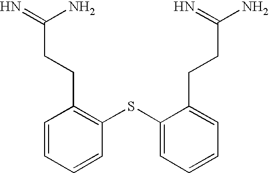

To a stirred suspension of ammonium chloride (0.22 g, 4.10 mmol) in toluene (5.0 mL) was added trimethyl aluminum in toluene (2.05 mL of 2.0 M solution, 4.10 mmol) at 0° C. The mixture was stirred at ambient temperature for 2 hours, followed by the addition of 2-(6-(2-cyanoethyl)phenyl)thio-1-(2-cyanoethyl)benzene (0.12 g, 0.41 mmol). The mixture was stirred at 110° C. for 16 hours and poured to a chloroform and silica gel mixture. The mixture was filtered and the solid pad was washed with methanol. The filtrate was dried in vacuo and the residue was dissolved in a mixed solvent of iso-propanol and acetone (20 mL/5 mL). The mixture was filtered and the filtrate was concentrated and dried in vacuo to afford 2-(6-(2-amidinoethyl)phenyl)thio-1-(2-amidinoethyl)benzene as a colorless solid in 82% yield (0.11 g): ¹H NMR (300 MHz, CD₃OD) δ 7.41 (dd, J=7.6, 1.3 Hz, 2H), 7.30 (ddd, J=7.6, 7.6, 1.3 Hz, 2H), 7.21 (ddd, J=7.6, 7.6, 1.3 Hz, 2H), 7.07 (dd, J=7.6, 1.3 Hz, 2H), 3.22 (t, J=7.0 Hz, 4H), 2.86 (t, J=7.0 Hz, 4H); ¹³C NMR (75 MHz, CD₃OD) δ 172.0, 140.7, 135.4, 133.2, 131.1, 129.3, 129.2, 34.1, 31.8; MS (ES+) m/z 327.3 (M+1).

### Example 6.1

### Synthesis of 2-(6-(amidinomethyl)phenyl)thio-1-(amidinomethyl)benzene

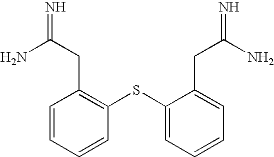

Following the procedure as described in Example 6, making non-critical variations using 2-(6-(cyanomethyl)phenyl)thio-1-(cyanomethyl)benzene to replace 2-(6-(2-cyanoethyl)phenyl)thio-1-(2-cyanoethyl)benzene, 2-(6-(amidinomethyl)phenyl)thio-1-(amidinomethyl)benzene was obtained as a colorless solid in 90% yield: ¹H NMR (300 MHz, CD₃OD) δ 7.53-7.32 (m, 6H), 7.18 (dd, J=7.6, 1.5 Hz, 2H), 4.08 (s, 4H); ¹³C NMR (75 MHz, CD₃OD) δ 170.9, 135.8, 134.3, 133.6, 132.5, 130.9, 130.0, 37.8; MS (ES+) m/z 299.3 (M+1).

### Example 6.2

### Synthesis of 2-(6-(3-amidinopropyl)phenyl)thio-1-(3-amidinopropyl)benzene

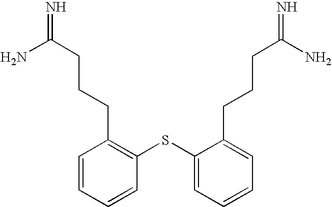

Following the procedure as described in Example 6, making non-critical variations using 2-(6-(3-cyanopropyl)phenyl)thio-1-(3-cyanopropyl)benzene to replace 2-(6-(2-cyanoethyl)phenyl)thio-1-(2-cyanoethyl)benzene, 2-(6-(3-amidinopropyl)phenyl)thio-1-(3-amidinopropyl)benzene was obtained as a colorless solid in 67% yield: ¹H NMR (300 MHz, CD₃OD) δ 7.34 (dd, J=7.6, 1.5 Hz, 2H), 7.27 (ddd, J=7.6, 7.6, 1.5 Hz, 2H), 7.16 (ddd, J=7.6, 7.6, 1.5 Hz, 2H), 7.05 (d, J=7.6 Hz, 2H), 2.89 (t, J=7.6 Hz, 4H), 2.54 (t, J=7.6 Hz, 4H), 2.10-1.96 (m, 4H); ¹³C NMR (75 MHz, CD₃OD) δ 172.8, 142.6, 135.4, 133.1, 131.2, 129.0, 128.7, 34.1, 33.3, 28.9; MS (ES+) m/z 344.4 (M+1).

### Example 7

### Synthesis of 2-(6-(2-(cyanoamidino)ethyl)phenyl)thio-1-(2-(cyanoamidino)ethyl)benzene

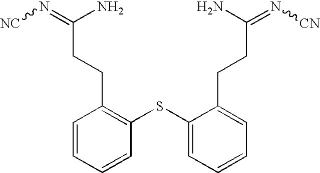

A solution of 2-(6-(2-cyanoethyl)phenyl)thio-1-(2-cyanoethyl)benzene (0.35 g, 1.20 mmol) in ethanol (10 mL) was saturated with dry hydrogen chloride gas at 0° C. The mixture was stirred at ambient temperature for 16 hours and concentrated in vacuo. The residue was dissolved in methanol (2 mL) and the solution was poured into a cold potassium carbonate solution. The mixture was extracted with ethyl acetate (3×30 mL) and the combined organic layers was dried over anhydrous sodium sulfate and filtered. The filtrate was concentrated in vacuo. The residue was dissolved in methanol (10 mL), followed by the addition of cyanamide (0.25 g, 6.00 mmol). The mixture was stirred at ambient temperature for 16 hours and concentrated in vacuo. The residue was purified by column chromatography (ethyl acetate/hexane, 2/1) to afford 2-(6-(2-(cyanoamidino)ethyl)phenyl)thio-1-(2-(cyanoamidino)ethyl)benzene as an colorless solid in 57% yield (0.26 g): mp 188-190° C.; ¹H NMR (300 MHz, CD₃OD) δ 7.35 (d, J=7.3 Hz, 2H), 7.25 (ddd, J=7.3, 7.3, 1.5 Hz, 2H), 7.17 (ddd, J=7.6, 7.6, 1.5 Hz, 2H), 7.05 (dd, J=7.6, 1.2 Hz, 2H), 3.24-3.09 (br, 4H), 2.94-2.61 (br, 4H); ¹³C NMR (75 MHz, CD₃OD) δ 141.6, 135.5, 133.1, 131.2, 129.0, 128.9, 36.5, 32.1; MS (ES+) m/z 377.3 (M+1).

### Example 7.1

### Synthesis of 2-(6-((cyanoamidino)methyl)phenyl)thio-1-((cyanoamidino)methyl)benzene

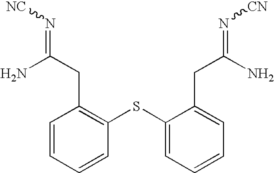

Following the procedure as described in Example 7, making non-critical variations using 2-(6-(cyanomethyl)phenyl)thio-1-(cyanomethyl)benzene to replace 2-(6-(2-cyanoethyl)phenyl)thio-1-(2-cyanoethyl)benzene, 2-(6-((cyanoamidino)methyl)phenyl)thio-1-((cyanoamidino)methyl)benzene was obtained as a colorless solid in 9% yield: ¹H NMR (300 MHz, CD₃OD) δ 7.40-7.30 (m, 4H), 7.26 (ddd, J=7.6, 7.6, 2.0 Hz, 2H), 7.16 (d, J=7.6 Hz, 2H), 3.96 (br, 4H); MS (ES+) m/z 349.3 (M+1).

### Example 7.2

### Synthesis of 2-(6-(3-(cyanoamidino)propyl)phenyl)thio-1-(3-(cyanoamidino)propyl)benzene

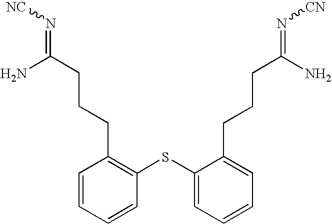

Following the procedure as described in Example 7, making non-critical variations using 2-(6-(3-cyanopropyl)phenyl)thio-1-(3-cyanopropyl)benzene to replace 2-(6-(2-cyanoethyl)phenyl)thio-1-(2-cyanoethyl)benzene, 2-(6-(3-(cyanoamidino)propyl)phenyl)thio-1-(3-(cyanoamidino)propyl)benzene was obtained as a colorless solid in 22% yield: mp 155-157° C.; ¹H NMR (300 MHz, CD₃OD) δ 7.31 (d, J=7.3 Hz, 2H), 7.23 (dd, J=7.3, 7.3 Hz, 2H), 7.10 (d, J=7.6, 2H), 7.04 (d, J=7.6 Hz, 2H), 2.85 (t, J=7.9 Hz, 4H), 2.65-2.34 (br, 4H), 2.00 (m, 4H); ¹³C NMR (75 MHz, CD₃OD) δ 143.1, 135.6, 133.1, 131.1, 128.8, 128.4, 35.5, 34.4, 28.9; MS (ES+) m/z 405.3 (M+1).

### Example 8

### Synthesis of 1,1′-(2,2′-thiobis(2,1-phenylene))dithiourea

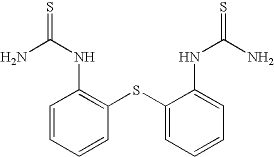

A. A mixture of 2,2′-thiodianiline (0.43 g, 2.00 mmol) and benzoyl isothiocyanate (0.65 g, 4.00 mmol) in acetone (15.0 mL) was stirred at ambient temperature for 1 h. The white precipitation was collected and dried in air to afford N,N′-(2,2′-thiobis(2,1-phenylene)bis(azanediyl))bis(thioxomethylene)dibenzamide in 94% yield (1.04 g): ¹H NMR (300 MHz, DMSO-d₆) δ 11.40 (s, 2H), 11.39-11.03 (m, 2H), 7.92-7.85 (m, 4H), 7.63-7.54 (m, 2H), 7.50-7.36 (m, 6H), 7.34-7.21 (m, 4H), 7.16-7.08 (m, 2H), 4.95 (d, J=5.5 Hz, 4H); MS (ES+) m/z 543.1 (M+1).

B. To a stirred suspension of N,N′-(2,2′-thiobis(2,1-phenylene)bis(azanediyl))bis(thioxo-methylene)dibenzamide (0.88 g, 1.61 mmol) was added a solution of sodium hydroxide (0.3 g) in water (15.0 mL). The mixture was stirred at 60° C. for 1 h and concentrated to dryness. The residue was purified by column chromatography to afford 1,1′-(2,2′-thiobis(2,1-phenylene))dithiourea as a colorless solid in 75% yield (0.41 g): ¹H NMR (300 MHz, DMSO-d₆) δ 9.20 (s, 2H), 7.57-7.47 (m, 2H), 7.34-7.18 (m, 4H), 7.17-7.01 (m, 6H); MS (ES+) m/z 335.1 (M+1).

### Example 9

In a similar manner as described above utilizing the appropriately substituted starting materials, the following compounds of the invention were prepared:
2-(2′-carbamimidoylsulfanylmethyl-biphenyl-2-ylmethyl)-isothiourea;
2-[2-({[amino(imino)methyl]thio}methyl)-5-chlorophenoxy]-5-fluorobenzyl imidothiocarbamate; and
4,4-diisothiourea benzophenone.

### Biological Assays

Various techniques are known in the art for testing the activity of compounds of the invention. In order that the invention described herein may be more fully understood, the following biological assays are set forth. It should be understood that these examples are for illustrative purposes only and are not to be construed as limiting this invention in any manner.

### Biological Example 1

### DMT1 Activity Assay (In Vitro Assay)

This example discloses various in vitro assay for testing and profiling test agents against DMT1 stably expressed in cells of either an endogenous or recombinant origin. These assays can use stable cell lines overexpressing DMT1 or intestinal cells and intestinal tissue expressing endogenous DMT1. DMT1 function could also be assessed in other cell types that express DMT1. Of greatest relevance would be the erythrocytes (e.g. K562 cells) or hepatocytes (e.g. HepG3).

DMT1 function can be assessed in a number of ways, including monitoring fluorescence changes of an iron fluorophore (e.g. calcein), monitoring uptake of radiolabelled iron (55Fe or ⁵⁹Fe) (Picard et al., J. Biol. Chem., 2000, 275(46):35738-45 and Wetli et al., Chem. Biol. 2006 September; 13(9):965-72), or by assessing the current or transport of iron and other metals into the cells or tissues using standard electrophysiological techniques (Gunshin et al., Nature, 1997, 388(6641):482-8.).

Variations of these assays involve alterations of incubation times, the iron status of the cells and tissues (which may be modulated by chemical chelators or by harvesting from iron deficient animals), the metal cation detected and the pH of the reaction can generally be made by conventional techniques known to those skilled in the art.

### Biological Example 2

### In Vivo Assay for Treatment of Iron Disorders

This test measures the efficacy of compounds of the invention in blocking ferrous iron uptake in the duodenum in rats. The animals were rendered iron deficient by feeding an iron deficient diet for 3 weeks, which causes a marked decrease in serum iron and transferrin saturation. As a result of the iron deficiency, DMT1 expression in the duodenum is upregulated. The test animals were then given an oral bolus (or an “iron challenge”) of ferrous iron at 1 mg/kg resulting in a 20-fold increase in serum iron 1 hour post challenge. It was observed that when test animals were dosed with compound 1 hour prior to the iron challenge, there was a substantial reduction in the increase in serum iron level 1 hour post iron challenge. Compounds of the present invention were shown to be efficacious within a range of 30 mg/Kg and 0.1 mg/Kg.

Representative compounds of the invention, when tested in the above assay, demonstrated an IC₅₀ (nM) activity level as set forth below in Table 1 wherein “A” refers to an IC₅₀ activity level of from 1 nM to 10 nM, “B” refers to an IC₅₀ activity level from 10 nM to 100 nM, “C” refers to an IC₅₀ activity level from 100 nM to 1.0 μM, and “D” refers to an IC₅₀ activity level equal to or greater than 1.0 μM. The Example numbers provided in Table 1 correspond to the Examples herein:

TABLE 1

| Example | | IC₅₀ Activity |
|---|---|---|
| No. | Compound Name | Level |
| 1 | 2-(1-{2-[2-(1-carbamimidoylsulfanylethyl)phenoxy]- | D |
| | phenyl}ethyl)-isothiourea | |
| 1.1 | 2-[2-(2-carbamimidoylsulfanylmethylphenylsulfanyl)benzyl]- | C |
| | isothiourea dihydrobromide | |
| 1.2 | 2-[2-(2-carbamimidoylsulfanylmethyl-4- | C |
| | fluorophenylsulfanyl)benzyl]-isothiourea | |
| | dihydrobromide | |
| 1.3 | 2-[2-(2-carbamimidoylsulfanylmethyl-5- | D |
| | methylphenylsulfanyl)-benzyl]isothiourea | |
| | dihydrobromide | |
| 1.4 | 2-[2-(2-carbamimidoylsulfanylmethyl-4- | D |
| | methoxyphenylsulfanyl)-benzyl]isothiourea | |
| | dihydrobromide | |
| 1.5 | 2-[2-(2-carbamimidoylsulfanylmethyl-5- | D |
| | methylphenylsulfanyl)-5-fluorobenzyl]isothiourea | |
| | dihydrobromide | |
| 1.6 | 2-[2-(2-carbamimidoylsulfanylmethyl-4- | C |
| | methoxyphenylsulfanyl)-5-fluorobenzyl]isothiourea | |
| | dihydrobromide | |
| 1.7 | 2-[2-(2-carbamimidoylsulfanylmethyl-5-chloro- | C |
| | phenylsulfanyl)-5-fluoro-benzyl]-isothiourea | |
| | dihydrobromide | |
| 1.8 | 2-[2-(2-carbamimidoylsulfanylmethyl-6- | D |
| | methylphenylsulfanyl)-benzyl]isothiourea | |
| | dihydrobromide | |
| 1.9 | 2-[2-(2-carbamimidoylsulfanylmethyl-4,5- | C |
| | difluorophenylsulfanyl)-benzyl]isothiourea | |
| | dihydrobromide | |
| 1.10 | 2-[2-(2-carbamimidoylsulfanylmethyl-4- | C |
| | methylphenylsulfanyl)-benzyl]isothiourea | |
| | dihydrobromide | |
| 1.11 | 2-[2-(2-carbamimidoylsulfanylmethyl-5- | C |
| | chlorophenylsulfanyl)-benzyl]isothiourea | |
| | dihydrobromide | |
| 1.12 | 2-[2-(2-carbamimidoylsulfanylmethyl-5- | C |
| | fluorophenylsulfanyl)benzyl]-isothiourea | |
| | dihydrobromide | |
| 1.13 | 2-[2-(1-carbamimidoylsulfanylmethylnaphthalen-2- | D |
| | ylsulfanyl)benzyl]-isothiourea dihydrobromide | |
| 1.14 | 2-[2-(1-carbamimidoylsulfanylmethylnaphthalen-2- | D |
| | ylsulfanyl)-5-fluorobenzyl]isothiourea dihydrobromide | |
| 1.15 | 2-[2-(2-carbamimidoylsulfanylmethyl-5- | C |
| | fluorophenylsulfanyl)-5-fluorobenzyl]isothiourea | |
| | dihydrobromide | |
| 1.16 | 2-[2-(2-carbamimidoylsulfanylmethyl-4,5- | C |
| | difluorophenylsulfanyl)-5-fluorobenzyl]isothiourea | |
| | dihydrobromide | |
| 1.17 | 2-[2-(2-carbamimidoylsulfanylmethyl-4- | D |
| | fluorophenylsulfanyl)-3-nitrobenzyl]isothiourea | |
| | dihydrobromide | |
| 1.18 | 2-[2-(2-carbamimidoylsulfanylmethyl-4- | D |
| | fluorophenylsulfanyl)-5- | |
| | (trifluoromethyl)benzyl]isothiourea dihydrobromide | |
| 1.19 | 2-[2-(2-carbamimidoylsulfanylmethylphenylsulfanyl)-5- | C |
| | aminobenzyl]-isothiourea trihydrobromide | |
| 1.20 | 2-[2-(2-carbamimidoylsulfanylmethyl-4- | C |
| | chlorophenylsulfanyl)-5-fluorobenzyl]isothiourea | |
| | dihydrobromide | |
| 1.21 | 2-[2-(2-carbamimidoylsulfanylmethylphenylsulfanyl)-5- | D |
| | ethylaminobenzyl]isothiourea trihydrobromide | |
| 1.22 | 2-[2-(2-carbamimidoylsulfanylmethyl-4- | D |
| | chlorophenylsulfanyl)-5-chlorobenzyl]isothiourea | |
| | dihydrobromide | |
| 1.23 | 2-[2-(2-carbamimidoylsulfanylethyl-4-fluorophenylsulfanyl)- | C |
| | 5-fluorobenzyl]isothiourea dihydrobromide | |
| 1.24 | 2-[2-(2-carbamimidoylsulfanylethyl-4-chlorophenylsulfanyl)- | C |
| | 5-fluorobenzyl]isothiourea dihydrobromide | |
| 1.25 | 2-[2-(2-carbamimidoylsulfanylethylphenylsulfanyl)- | D |
| | benzyl]isothiourea dihydrobromide | |
| 1.26 | 2-[2-(2-carbamimidoylsulfanylmethylphenylsulfanyl)-5- | C |
| | chlorobenzyl]-isothiourea dihydrobromide | |
| 1.27 | 2-[2-(2-carbamimidoylsulfanylmethylphenylsulfanyl)-5- | D |
| | (trifluoromethyl)benzyl]isothiourea dihydrobromide | |
| 1.28 | 2,2′-(methylazanediyl)bis(2,1-phenylene)bis- | C |
| | (methylene)dicarbamimidothioate trihydrobromide | |
| 1.29 | 2-[2-(2-methylcarbamimidoylsulfanylmethylphenyl- | D |
| | sulfanyl)benzyl]-methylisothiourea dihydrobromide | |
| 1.30 | 2-[2-(2-carbamimidoylsulfanylmethyl-4- | C |
| | fluorophenylsulfanyl)-5- | |
| | (methylsulfonyl)benzyl]isothiourea dihydrobromide | |
| 1.31 | 2-[2-(2-carbamimidoylsulfanylmethylphenylsulfanyl)benzyl]- | D |
| | isothiourea dihydrobromide | |
| 2 | 2-[2-(2-carbamimidoylsulfanylmethyl-4-fluoro- | C |
| | phenylsulfanyl)-5-fluoro-benzyl]-isothiourea | |
| | dihydrobromide | |
| 2.1 | 2-{[2-({[amino(imino)methyl]thio}methyl)-4- | B |
| | fluorophenyl]thio}-5-nitrobenzyl imidothiocarbamate | |
| | dihydrobromide | |
| 2.2 | 2-({2-({[amino(imino)methyl]thio}methyl)-4- | C |
| | [(dimethylamino)sulfonyl]phenyl}thio)-5-fluorobenzyl | |
| | imidothiocarbamate dihydrobromide | |
| 2.3 | 2-[2-(2-carbamimidoylsulfanylmethylphenylsulfonyl)benzyl]- | D |
| | isothiourea dihydrobromide | |
| 2.4 | 2-[2-(2-carbamimidoylsulfanylmethyl-4- | D |
| | fluorophenylsulfonyl)-5-fluorobenzyl]isothiourea | |
| | dihydrobromide | |
| 2.5 | (4-{[2-({[amino(imino)methyl]thio}methyl)-4- | C |
| | fluorophenyl]thio}-3-thienyl)methyl | |
| | imidothiocarbamate dihydrochloride | |
| 2.6 | (2-{[2-({[amino(imino)methyl]thio}methyl)-4- | D |
| | fluorophenyl]thio}-3-thienyl)methyl | |
| | imidothiocarbamate dihydrochloride | |
| 2.7 | 2-{[2-({[amino(imino)methyl]thio}methyl)-4- | D |
| | fluorophenyl]thio}-4-(methylsulfonyl)benzyl | |
| | imidothiocarbamate dihydrochloride | |
| 2.8 | 2-{[2-({[amino(imino)methyl]thio}methyl)-4- | D |
| | chlorophenyl]thio}-5-cyanobenzyl | |
| | imidothiocarbamate dihydrobromide | |
| 2.9 | 2-[2-({[amino(imino)methyl]thio}methyl)-5-chlorophenoxy]-4- | D |
| | nitrobenzyl imidothiocarbamate dihydrobromide | |
| 2.10 | 2-[2-(2-carbamimidoylsulfanylmethyl-3-chlorophenoxy)-5- | D |
| | fluorobenzyl]isothiourea dihydrobromide | |
| 2.11 | 2-[2-(2-carbamimidoylsulfanylmethyl-4-chlorophenoxy)-5- | C |
| | fluorobenzyl]isothiourea dihydrobromide | |
| 2.12 | 2-[2-(2-carbamimidoylsulfanylmethyl-5-fluorophenoxy)-5- | C |
| | fluorobenzyl]isothiourea dihydrobromide | |
| 2.13 | 2-[2-(2-carbamimidoylsulfanylmethyl-4-fluorophenoxy)-5- | C |
| | fluorobenzyl]isothiourea dihydrobromide | |
| 2.14 | 2-[2-({[amino(imino)methyl]thio}methyl)-4-fluorophenoxy]-5- | C |
| | nitrobenzyl imidothiocarbamate dihydrobromide | |
| 2.15 | 2-[2-(2-carbamimidoylsulfanylmethylphenoxy)benzyl]- | D |
| | isothiourea dihydrobromide | |
| 2.16 | 2-[2-({[amino(imino)methyl]thio}methyl)-4- | D |
| | chlorophenoxy]benzyl imidothiocarbamate | |
| | dihydrobromide | |
| 2.17 | 2-[2-({[amino(imino)methyl]thio}methyl)-3- | D |
| | chlorophenoxy]benzyl imidothiocarbamate | |
| | dihydrobromide | |
| 2.18 | 2-[2-({[amino(imino)methyl]thio}methyl)-5- | D |
| | chlorophenoxy]benzyl imidothiocarbamate | |
| | dihydrobromide | |
| 2.19 | 2-[2-({[amino(imino)methyl]thio}methyl)-5-chlorophenoxy]-5- | D |
| | nitrobenzyl imidothiocarbamate dihydrobromide | |
| 2.20 | 2-[2-({[amino(imino)methyl]thio}methyl)-4-chlorophenoxy]-5- | D |
| | nitrobenzyl imidothiocarbamate dihydrobromide | |
| 2.21 | 2-[2-({[amino(imino)methyl]thio}methyl)-4- | C |
| | fluorophenoxy]benzyl imidothiocarbamate | |
| | dihydrobromide | |
| 2.23 | biphenyl-2,2′-6,6′-tetrayltetrakis(methylene) | D |
| | tetracarbamimidothioate tetrahydrobromide | |
| 2.24 | dimethyl 6,6′-bis(carbamimidoylthiomethyl)biphenyl-2,2′- | D |
| | dicarboxylate dihydrobromide | |
| 2.25 | 2-(6-(amidinothiomethyl)phenyl)carbonyl-1- | C |
| | (amidinothiomethyl)benzene dihydrobromide | |
| 2.26 | 2-(6-(amidinothiomethyl)phenyl)carbonyl-1- | C |
| | (amidinothiomethyl)-5-fluorobenzene dihydrobromide | |
| 2.27 | 2-(6-(amidinothiomethyl)phenyl)carbonyl-1- | D |
| | (amidinothiomethyl)-4-fluorobenzene dihydrobromide | |
| 2.28 | 2-(6-(amidinothiomethyl)phenyl)carbonyl-1- | D |
| | (amidinothiomethyl)-4-chlorobenzene | |
| | dihydrobromide dihydrobromide | |
| 2.29 | 2-(6-(amidinothiomethyl)phenyl)carbonyl-1- | D |
| | (amidinothiomethyl)-5-chlorobenzene | |
| | dihydrobromide | |
| 2.30 | 2-[2-({[amino(imino)methyl]thio}methyl)-5-chlorophenoxy]-5- | C |
| | fluorobenzyl imidothiocarbamate dihydrobromide | |
| 3 | 2-(6-((aminoamidino)thiomethyl)phenyl)thio-1- | D |
| | ((aminoamidino)thiomethyl)benzene dihydrobromide | |
| 5 | 1-(2-(2-(guanidinomethyl)phenylthio)benzyl)guanidine (4- | D |
| | methylbenzenesulfonate) | |
| 6 | 2-(6-(2-amidinoethyl)phenyl)thio-1-(2-amidinoethyl)benzene | D |
| 6.1 | 2-(6-(amidinomethyl)phenyl)thio-1-(amidinomethyl)benzene | D |
| 6.2 | 2-(6-(3-amidinopropyl)phenyl)thio-1-(3- | D |
| | amidinopropyl)benzene | |
| 7 | 2-(6-(2-(cyanoamidino)ethyl)phenyl)thio-1-(2- | D |
| | (cyanoamidino)ethyl)benzene | |
| 7.1 | 2-(6-((cyanoamidino)methyl)phenyl)thio-1- | D |
| | ((cyanoamidino)methyl)benzene | |
| 7.2 | 2-(6-(3-(cyanoamidino)propyl)phenyl)thio-1-(3- | D |
| | (cyanoamidino)propyl)benzene | |
| 8 | 1,1′-(2,2′-thiobis(2,1-phenylene))dithiourea | D |
| 9 | 2-(2′-carbamimidoylsulfanylmethyl-biphenyl-2-ylmethyl)- | D |
| | isothiourea | |

A variation of this assay can be used for longer term studies. In this variation, animals are again rendered iron deficient by feeding of an iron deficient diet for 3 weeks. Then animals are switched back to an iron replete diet, while receiving a daily dose of either vehicle or a compound described herein. The vehicle animals recover their iron status, as measured by serum iron and other iron indices, after 13 days. The drug treated animals, however, do not recover in this timeframe, as the compound is blocking the uptake of dietary iron. Other parameters that can be measured in both models include transferrin saturation, haemoglobin, hematocrit, liver iron and ferritin. More detailed assays can involve the use of radioactive metals as opposed to a bolus of ferrous iron. Multiple metals transported by DMT1 can be used to judge specificity of compound on cation uptake by DMT1, if any.

Genetic rat models of iron overload offers another format to show efficacy of DMT1 inhibitors in preventing further iron loading as development proceeds. These models are applicable to variety of human iron overload disorders such as hereditary hemochromatosis (Levy et al, Blood, 1999, 94:9-11, 1999), juvenile hemochromatosis (Huang et al, J. Clin. Invest., 2005 115:2187-2191), beta-2-microglobulin (de Sousa et al., Immun. Lett., 1994, 39:105-111, 1994), thalassemia (Ciavatta et al., Proc. Nat. Acad. Sci., 1995, 92: 9259-9263), hypotransferrinmia (Craven et. al., Proc. Nat. Acad. Sci., 1987, USA. 84(10):3457-61) and other hypochromic microcytic anemias.

In these models, the knock-out animals above are bred and treated with compound as they develop. Compound efficacy can be assessed by measuring reduced iron flux via the duodenum in a radioactive flux study or by monitoring whether chronic exposure to compounds cause a decrease in the amount of iron loading, as judged by serum iron, transferrin saturation, ferritin and liver iron. These models can be used with an iron bolus, or challenge, as above or iron may be absorbed from the diet. Where appropriate, a model of transfusional iron overload can be created in the rodent by transfusion of iron from another animal in order to exacerbate the iron overload is as seen clinically in the treatment of thalassemia.

All of the U.S. patents, U.S. patent application publications, U.S. patent applications, foreign patents, foreign patent applications and non-patent publications referred to in this specification are incorporated herein by reference in their entireties.

Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

What is claimed is:

1. A compound of formula (I): 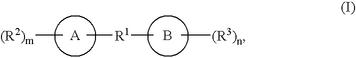
wherein:
n and m are each independently 1, 2, 3, 4, 5, 6 or 7; 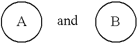
are each independently aryl or heteroaryl;
R¹ is a direct bond, —O—, —S(O)ₚ— (where p is 0, 1 or 2), —C(R⁴)₂—, —C(O)— or —N(R⁴)—;
at least one R² and at least one R³ is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
and the other R²'s and R³'s, if present, are each independently selected from the group consisting of alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶⁰—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, optionally substituted aryl, optionally substituted aralkyl, optionally substituted cycloalkyl, optionally substituted heterocyclyl, optionally substituted heteroaryl, or optionally substituted heteroaralkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl;
as a stereoisomer, enantiomer, tautomer thereof or mixtures thereof;
or a pharmaceutically acceptable salt, solvate or prodrug thereof.

2. The compound of claim 1, wherein the compound is a compound of formula (Ia): 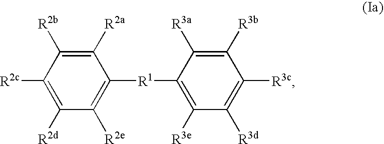
wherein:
R¹ is a direct bond, —O—, —S(O)ₚ— (where p is 0, 1 or 2), —C(R⁴)₂—, —C(O)— or —N(R⁴)—;
R^{2a}, R^{2b}, R^{2c}, R^{2d} and R^{2e} are each independently selected from the group consisting of hydrogen, alkyl, halo, haloalkyl, —R⁶—OR⁷, —R⁶—CN, —R⁸—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—O—C(O)—N═C[N(R⁴)(R⁵)]N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2 and wherein at least one of R², R^{2b}, R^{2c}, R^{2d} and R^{2e} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{3a}, R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁸—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵ wherein each t is independently 1 or 2 and each p is 0, 1 or 2 and wherein at least one of R^{3a}, R^{3b}, R^{3c}, R^{3d} and R^{3e} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
each R⁴ and R⁵ is independently hydrogen, alkyl, optionally substituted aryl, optionally substituted aralkyl, optionally substituted cycloalkyl, optionally substituted heterocyclyl, optionally substituted heteroaryl, or optionally substituted heteroaralkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

3. The compound of claim 2 wherein:
R¹ is a direct bond;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

4. The compound of claim 3 wherein:
R¹ is a direct bond;
R^{2a} and R^{3a} are the same and selected from the group consisting of —R⁶—S—C(═NR⁴)N(R⁴)R⁵, —R⁶—O—C(═NR⁴)N(R⁴)R⁵, —R⁶—C(═NR⁴)N(R⁴)R⁵ and —R⁶—N(R⁷)C(═NR⁴)N(R⁴)R⁵;
R^{2e} and R^{3e} are the same and selected from the group consisting of hydrogen, alkyl, —R⁶—C(O)OR⁸, —R⁶—C(O)N(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵ and —R⁶—N(R⁷)C(═NR⁴)N(R⁴)R⁵;
R^{2b}, R^{2c} and R^{2d} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

5. The compound of claim 4 wherein:
R¹ is a direct bond;
R^{2a} and R^{3a} are both —R⁶—S—C(═NR⁴)N(R⁴)R⁵;
R^{2e} and R^{3e} are the same and selected from the group consisting of hydrogen, alkyl, —R⁶—C(O)OR⁸ and —R⁶—S—C(═NR⁴)N(R⁴)R⁵;
R^{2b}, R^{2c} and R^{2d} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

6. The compound of claim 5 wherein:
R¹ is a direct bond;
R^{2a} and R^{3a} are both —R⁶—S—C(═NR⁴)N(R⁴)R⁵;
R^{2e} and R^{3e} are the same and selected from the group consisting of hydrogen, alkyl, —R⁶—C(O)OR⁸ and —R⁶—S—C(═NR⁴)N(R⁴)R⁵;
R^{2b}, R^{2c} and R^{2d} are each independently selected from the group consisting of hydrogen, alkyl, halo and haloalkyl;
R^{3b}, R^{3c} and R^{3d} are each independently selected from the group consisting of hydrogen, alkyl, halo and haloalkyl;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R⁸ is hydrogen or alkyl.

7. The compound of claim 6 selected from the group consisting of:
2-(2′-carbamimidoylsulfanylmethyl-biphenyl-2-ylmethyl)-isothiourea;
(6,6′-dimethylbiphenyl-2,2′-diyl)bis(methylene)dicarbamimidothioate dihydrobromide;
biphenyl-2,2′,6,6′-tetrayltetrakis(methylene)tetracarbamimidothioate; and
dimethyl 6,6′-bis(carbamimidoylthiomethyl)biphenyl-2,2′-dicarboxylate.

8. The compound of claim 2 wherein:
R¹ is —O—;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

9. The compound of claim 8 wherein:
R¹ is —O—;
R^{2a} and R^{3a} are the same and selected from the group consisting of —R⁶—S—C(═NR⁴)N(R⁴)R⁵, —R⁶— —C(═NR⁴)N(R⁴)R⁵, —R⁶—C(═NR⁴)N(R⁴)R⁵ and —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

10. The compound of claim 9 wherein:
R¹ is —O—;
R^{2a} and R^{3a} are both —R⁶—S—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —N(R⁸)S(O)ₜR⁹, —S(O)ₜOR⁹, —S(O)ₚR⁸ and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

11. The compound of claim 10 wherein:
R¹ is —O—;
R^{2a} and R^{3a} are both —R⁶—S—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂ and —R⁶—N(R⁸)₂;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂ and —R⁶—N(R⁸)₂;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl.

12. The compound of claim 11 selected from the group consisting of:
2-[2-(2-carbamimidoylsulfanylmethyl-phenoxy)-benzyl]-isothiourea;
2-(1-{2-[2-(1-carbamimidoylsulfanyl-ethyl)-phenoxy]-phenyl}-ethyl)-isothiourea;
2-[2-({[amino(imino)methyl]thio}methyl)-4-fluorophenoxy]-5-nitrobenzyl imidothiocarbamate;
2-[2-({[amino(imino)methyl]thio}methyl)-5-chlorophenoxy]-4-nitrobenzyl imidothiocarbamate;
2-[2-({[amino(imino)methyl]thio}methyl)-5-chlorophenoxy]-5-fluorobenzyl imidothiocarbamate;
2-[2-(2-carbamimidoylsulfanylmethyl-3-chlorophenoxy)-5-fluorobenzyl]isothiourea;
2-[2-(2-carbamimidoylsulfanylmethyl-4-chlorophenoxy)-5-fluorobenzyl]isothiourea;
2-[2-({[amino(imino)methyl]thio}methyl)-4-chlorophenoxy]benzyl imidothiocarbamate;
2-[2-({[amino(imino)methyl]thio}methyl)-3-chlorophenoxy]benzyl imidothiocarbamate;
2-[2-({[amino(imino)methyl]thio}methyl)-5-chlorophenoxy]benzyl imidothiocarbamate;
2-[2-({[amino(imino)methyl]thio}methyl)-5-chlorophenoxy]-5-nitrobenzyl imidothiocarbamate;
2-[2-({[amino(imino)methyl]thio}methyl)-4-chlorophenoxy]-5-nitrobenzyl imidothiocarbamate;
2-[2-(2-carbamimidoylsulfanylmethyl-5-fluorophenoxy)-5-fluorobenzyl]isothiourea;
2-[2-(2-carbamimidoylsulfanylmethyl-4-fluorophenoxy)-5-fluorobenzyl]isothiourea; and
2-[2-({[amino(imino)methyl]thio}methyl)-4-fluorophenoxy]benzyl imidothiocarbamate.

13. The compound of claim 2 wherein:
R¹ is —S(O)ₚ— (where p is 0, 1 or 2);
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R³)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—O—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

14. The compound of claim 13 wherein:
R¹ is —S(O)ₚ— (where p is 0, 1 or 2);
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

15. The compound of claim 14 wherein:
R¹ is —S(O)ₚ— (where p is 0, 1 or 2);
R^{2a} and R^{3a} are both —R⁶—S—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁸—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

16. The compound of claim 15 wherein:
R¹ is —S;
R^{2a} and R^{3a} are both —R⁶—S—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

17. The compound of claim 16 selected from the group consisting of:
2-[2-(2-carbamimidoylsulfanylmethyl-phenylsulfanyl)-benzyl]-isothiourea;
2-[2-(2-carbamimidoylsulfanylmethyl-4-fluoro-phenylsulfanyl)-5-fluoro-benzyl]-isothiourea;
2-[2-(2-carbamimidoylsulfanylmethyl-4-fluoro-phenylsulfanyl)-benzyl]-isothiourea;
2-[2-(2-carbamimidoylsulfanylmethyl-5-methyl-phenylsulfanyl)-benzyl]-isothiourea;
2-[2-(2-carbamimidoylsulfanylmethyl-4-methoxy-phenylsulfanyl)-benzyl]-isothiourea;
2-[2-(2-carbamimidoylsulfanylmethyl-5-methyl-phenylsulfanyl)-5-fluoro-benzyl]-isothiourea;
2-[2-(2-carbamimidoylsulfanylmethyl-4-methoxy-phenylsulfanyl)-5-fluoro-benzyl]-isothiourea;
2-[2-(2-carbamimidoylsulfanylmethyl-5-chloro-phenylsulfanyl)-5-fluoro-benzyl]-isothiourea;
2-[2-(2-carbamimidoylsulfanylmethyl-6-methylphenylsulfanyl)-benzyl]isothiourea;
2-[2-(2-carbamimidoylsulfanylmethyl-4,5-difluorophenylsulfanyl)-benzyl]isothiourea;
2-[2-(2-carbamimidoylsulfanylmethyl-4-methyl-phenylsulfanyl)-benzyl]-isothiourea;
2-[2-(2-carbamimidoylsulfanylmethyl-5-chloro-phenylsulfanyl)-benzyl]-isothiourea;
2-[2-(2-carbamimidoylsulfanylmethyl-5-fluoro-phenylsulfanyl)-benzyl]-isothiourea;
2-[2-(2-carbamimidoylsulfanylmethyl-5-fluoro-phenylsulfanyl)-5-fluorobenzyl]-isothiourea;
2-[2-(2-carbamimidoylsulfanylmethyl-4,5-difluorophenylsulfanyl)-5-fluorobenzyl]isothiourea;
2-{[2-({[amino(imino)methyl]thio}methyl)-4-fluorophenyl]thio}-3-nitrobenzyl imidothiocarbamate;
2-{[2-({[amino(imino)methyl]thio}methyl)-4-fluorophenyl]thio}-5-nitrobenzyl imidothiocarbamate;
2-[2-(2-carbamimidoylsulfanylmethyl-4-fluorophenylsulfanyl)-5-(trifluoromethyl)benzyl]isothiourea;
2-[2-(2-carbamimidoylsulfanylmethylphenylsulfanyl)-5-aminobenzyl]-isothiourea;
2-[2-(2-carbamimidoylsulfanylmethyl-4-chlorophenylsulfanyl)-5-fluorobenzyl]isothiourea;
2-[2-(2-carbamimidoylsulfanylmethylphenylsulfanyl)-5-ethylaminobenzyl]isothiourea;
2-[2-(2-carbamimidoylsulfanylmethyl-4-chlorophenylsulfanyl)-5-chlorobenzyl]isothiourea;
2-[2-(2-carbamimidoylsulfanylethyl-4-fluorophenylsulfanyl)-5-fluorobenzyl]isothiourea;
2-[2-(2-carbamimidoylsulfanylethyl-4-chlorophenylsulfanyl)-5-fluorobenzyl]isothiourea;
2-[2-(2-carbamimidoylsulfanylethylphenylsulfanyl)benzyl]isothiourea;
2-[2-(2-carbamimidoylsulfanylmethylphenylsulfanyl)-5-chlorobenzyl]-isothiourea;
2-[2-(2-carbamimidoylsulfanylmethylphenylsulfanyl)-5-(trifluoromethyl)benzyl]isothiourea;
2-[2-(2-methylcarbamimidoylsulfanylmethylphenylsulfanyl)benzyl]-methylisothiourea;
2-[2-(2-carbamimidoylsulfanylmethyl-4-fluorophenylsulfanyl)-5-(methylsulfonyl)benzyl]isothiourea;
2-({2-({[amino(imino)methyl]thio}methyl)-4-[(dimethylamino)sulfonyl]phenyl}thio)-5-fluorobenzyl imidothiocarbamate;
2-[2-(2-carbamimidoylsulfanylmethylphenylsulfanyl)benzyl]-isothiourea;
2-{[2-({[amino(imino)methyl]thio}methyl)-4-fluorophenyl]thio}-4-(methylsulfonyl)benzyl imidothiocarbamate; and
2-{[2-({[amino(imino)methyl]thio}methyl)-4-chlorophenyl]thio}-5-cyanobenzyl imidothiocarbamate.

18. The compound of claim 15 wherein:
R¹ is —S(O)₂—;
R^{2a} and R^{3a} are both —R⁶—S—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

19. The compound of claim 18 selected from the group consisting of:
2-[2-(2-carbamimidoylsulfanylmethylphenylsulfonyl)benzyl]-isothiourea; and
2-[2-(2-carbamimidoylsulfanylmethyl-4-fluorophenylsulfonyl)-5-fluorobenzyl]isothiourea.

20. The compound of claim 14 wherein:
R¹ is —S(O)ₚ— (where p is 0, 1 or 2);
R^{2a} and R^{3a} are both —R⁶—S—C(═NR⁴)N(R⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, Wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

21. The compound of claim 20 wherein:
R¹ is —S—;
R^{2a} and R^{3a} are both —R⁶—S—C(═NR⁴)N(R⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

22. The compound of claim 21 which is 2-(6-((aminoamidino)thiomethyl)phenyl)thio-1-((aminoamidino)thiomethyl)benzene.

23. The compound of claim 14 wherein:
R¹ is —S(O)ₚ— (where p is 0, 1 or 2);
R^{2a} and R^{3a} are both —R⁶—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂—R⁶—N(R⁸)₂—R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

24. The compound of claim 23 wherein:
R¹ is —S—;
R^{2a} and R^{3a} are both —R⁶—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

25. The compound of claim 24 selected from the group consisting of:
2-(6-(2-amidinoethyl)phenyl)thio-1-(2-amidinoethyl)benzene;
2-(6-(amidinomethyl)phenyl)thio-1-(amidinomethyl)benzene; and
2-(6-(3-amidinopropyl)phenyl)thio-1-(3-amidinopropyl)benzene.

26. The compound of claim 14 wherein:
R¹ is —S(O)ₚ— (where p is 0, 1 or 2);
R^{2a} and R^{3a} are both —R⁶—C(═NCN)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

27. The compound of claim 26 wherein:
R¹ is —S—;
R^{2a} and R^{3a} are both —R⁶—C(═NCN)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

28. The compound of claim 27 selected from the group consisting of:
2-(6-((cyanoamidino)methyl)phenyl)thio-1-((cyanoamidino)methyl)benzene;
2-(6-(2-(cyanoamidino)ethyl)phenyl)thio-1-(2-(cyanoamidino)ethyl)benzene; and
2-(6-(3-(cyanoamidino)propyl)phenyl)thio-1-(3-(cyanoamidino)propyl)benzene.

29. The compound of claim 14 wherein:
R¹ is —S(O)ₚ— (where p is 0, 1 or 2);
R^{2a} and R^{3a} are both —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

30. The compound of claim 29 wherein:
R¹ is —S—;
R^{2a} and R^{3a} are both —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

31. The compound of claim 30 which is 1-(2-(2-(guanidinomethyl)phenylthio)benzyl)guanidine.

32. The compound of claim 14 wherein:
R¹ is —S(O)ₚ— (where p is 0, 1 or 2);
R^{2a} and R^{3a} are both —R⁶—C(O)—N═C[N(R⁴)(R⁵)]N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

33. The compound of claim 32 wherein:
R¹ is —S—;
R^{2a} and R^{3a} are both —R⁶—C(O)—N═C[N(R⁴)(R⁵)]N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

34. The compound of claim 33 which is 2,2′-thiobis(N-(diaminomethylene)benzamide).

35. The compound of claim 2 wherein:
R¹ is —C(O)—;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

36. The compound of claim 35 wherein:
R¹ is —C(O)—;
R^{2a} and R^{3a} are each —R⁶—S—C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

37. The compound of claim 36 selected from the group consisting of:
2-(6-(amidinothiomethyl)phenyl)carbonyl-1-(amidinothiomethyl)benzene;
2-(6-(amidinothiomethyl)phenyl)carbonyl-1-(amidinothiomethyl)-5-fluorobenzene;
2-(6-(amidinothiomethyl)phenyl)carbonyl-1-(amidinothiomethyl)-4-fluorobenzene;
2-(6-(amidinothiomethyl)phenyl)carbonyl-1-(amidinothiomethyl)-4-chlorobenzene; and
2-(6-(amidinothiomethyl)phenyl)carbonyl-1-(amidinothiomethyl)-5-chlorobenzene.

38. The compound of claim 2 wherein:
R¹ is —C(O)—;
R^{2c} and R^{3c} are each —R⁶—S—C(═NR⁴)N(R⁴)R⁵;
R^{2a}, R^{2b}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3a}, R^{3b}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

39. The compound of claim 38 which is 4,4-diisothiourea benzophenone.

40. The compound of claim 2 wherein:
R¹ is —N(R⁴)—;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d} and R^{2e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

41. The compound of claim 40 wherein:
R¹ is —N(R⁴)—;
R^{2a} and R^{3a} are each —R⁶—S—C(═NR⁴)N(R⁴)R⁵;
R^{2b}, R^{2c}, R^{2d} and R^{2e} are each independently selected from the group consisting of hydrogen, alkyl, halo, haloalkyl, —OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

42. The compound of claim 41 which is 2,2-(methylazanediyl)bis(2,1-phenylene)bis(methylene)dicarbamimidothioate.

43. The compound of claim 1, wherein the compound is a compound of formula (Ib): 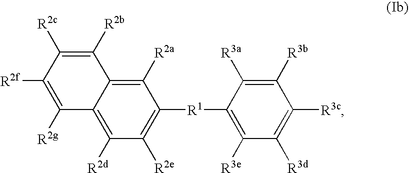
wherein:
R¹ is a direct bond, —O—, —S(O)ₚ— (where p is 0, 1 or 2), —C(R⁴)₂—, —C(O)— or —N(R⁴)—;
R^{2a}, R^{2b}, R^{2c}, R^{2d}, R^{2e}, R^{2f} and R^{2g} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2 and wherein at least one of R²,
R^{2b}, R^{2c}, R^{2d}, R^{2e}, R^{2f} and R^{2g} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{3a}, R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵ wherein each t is independently 1 or 2 and each p is 0, 1 or 2 and wherein at least one of R³,
R^{3b}, R^{3c}, R^{3d} and R^{3e} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
each R⁴ and R⁵ is independently hydrogen, alkyl, optionally substituted aryl, optionally substituted aralkyl, optionally substituted cycloalkyl, optionally substituted heterocyclyl, optionally substituted heteroaryl, or optionally substituted heteroaralkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

44. The compound of claim 43 wherein:
R¹ is —S(O)ₚ— (where p is 0, 1 or 2);
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d}, R^{2e}, R^{2f} and R^{2g} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

45. The compound of claim 44 wherein:
R¹ is —S(O)ₚ— (where p is 0, 1 or 2);
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b}, R^{2c}, R^{2d}, R^{2e}, R^{2f} and R^{2g} are each independently selected from the group consisting of hydrogen, alkyl, halo, haloalkyl, —R⁶—OR⁷, —R⁶—CN, —R⁸—NO₂, —R⁶—N(R⁸)₂, —R⁶—O—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

46. The compound of claim 45 wherein:
R¹ is —S(O)ₚ— (where p is 0, 1 or 2);
R^{2a} and R^{3a} are both —R⁶—S—C(═NR⁴)N(R⁴)R⁵;
R^{2b}, R^{2c}, R^{2d}, R^{2e}, R^{2f} and R^{2g} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

47. The compound of claim 46 wherein:
R¹ is —S—;
R^{2a} and R^{3a} are both —R⁶—S—C(═NR⁴)N(R⁴)R⁵;
R^{2b}, R^{2c}, R^{2d}, R^{2e}, R^{2f} and R^{2g} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

48. The compound of claim 47 selected from the group consisting of:
2-[2-(1-carbamimidoylsulfanylmethyl-naphthalen-2-ylsulfanyl)-benzyl]-isothiourea; and
2-[2-(1-carbamimidoylsulfanylmethylnaphthalen-2-ylsulfanyl)-5-fluorobenzyl]-isothiourea.

49. The compound of claim 1, wherein the compound is a compound of formula (Ic): 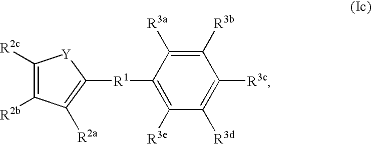
wherein:
Y is —O—, —S— or —N(R⁴)—;
R¹ is a direct bond, —O—, —S(O)ₚ— (where p is 0, 1 or 2), —C(R⁴)₂—, —C(O)— or —N(R⁴)—;
R^{2a}, R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2 and wherein at least one of R^{2a}, R^{2b} and R^{2c} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{3a}, R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵ wherein each t is independently 1 or 2 and each p is 0, 1 or 2 and wherein at least one of R³,
R^{3b}, R^{3c}, R^{3d} and R^{3e} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
each R⁴ and R⁵ is independently hydrogen, alkyl, optionally substituted aryl, optionally substituted aralkyl, optionally substituted cycloalkyl, optionally substituted heterocyclyl, optionally substituted heteroaryl, or optionally substituted heteroaralkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

50. The compound of claim 49 wherein:
Y is —S—;
R¹ is a direct bond, —O—, —S(O)ₚ— (where p is 0, 1 or 2), —C(O)— or —N(R⁴)—;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

51. The compound of claim 50 wherein:
Y is —S—;
R¹ is a direct bond, —O—, —S(O)ₚ— (where p is 0, 1 or 2), —C(O)— or —N(R⁴)—;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂—R⁶—N(R⁸)₂—R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

52. The compound of claim 51 wherein:
Y is —S—;
R¹ is —S(O)ₚ— (where p is 0, 1 or 2);
R^{2a} and R^{3a} are both —R⁶—S—C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

53. The compound of claim 52 wherein:
Y is —S—;
R¹ is —S—;
R^{2a} and R^{3a} are both —R⁶—S—C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

54. The compound of claim 53 which is (2-{[2-({[amino(imino)methyl]thio}methyl)-4-fluorophenyl]thio}-3-thienyl)methyl imidothiocarbamate.

55. The compound of claim 1, wherein the compound is a compound of formula (Id): 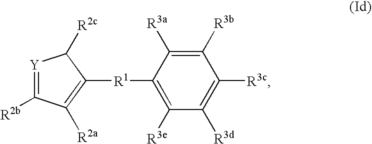
wherein:
Y is —O—, —S— or —N(R⁴)—;
R¹ is a direct bond, —O—, —S(O)ₚ— (where p is 0, 1 or 2), —C(R⁴)₂—, —C(O)— or —N(R⁴)—;
R^{2a}, R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2 and wherein at least one of R^{2a}, R^{2b} and R^{2c} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{3a}, R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵ wherein each t is independently 1 or 2 and each p is 0, 1 or 2 and wherein at least one of R³,
R^{3b}, R^{3c}, R^{3d} and R^{3e} is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
each R⁴ and R⁵ is independently hydrogen, alkyl, optionally substituted aryl, optionally substituted aralkyl, optionally substituted cycloalkyl, optionally substituted heterocyclyl, optionally substituted heteroaryl, or optionally substituted heteroaralkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

56. The compound of claim 55 wherein:
Y is —S—;
R¹ is a direct bond, —O—, —S(O)ₚ— (where p is 0, 1 or 2), —C(O)— or —N(R⁴)—;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁸—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁵—N(R⁷)—C(═NR⁴)N(R⁴)R⁵, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

57. The compound of claim 56 wherein:
Y is —S—;
R¹ is a direct bond, —O—, —S(O)ₚ— (where p is 0, 1 or 2), —C(O)— or —N(R⁴)—;
R^{2a} and R^{3a} are each independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

58. The compound of claim 57 wherein:
Y is —S—;
R¹ is —S(O)ₚ— (where p is 0, 1 or 2);
R^{2a} and R^{3a} are both —R⁶—S—C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, alkenyl, alkynyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

59. The compound of claim 58 wherein:
Y is —S—;
R¹ is —S—;
R^{2a} and R^{3a} are both —R⁶—S—C(═NR⁴)N(R⁴)R⁵;
R^{2b} and R^{2c} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
R^{3b}, R^{3c}, R^{3d} and R^{3e} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, and —S(O)ₜN(R⁸)₂, wherein each t is independently 1 or 2 and each p is 0, 1 or 2;
each R⁴ and R⁵ is independently hydrogen or alkyl;
each R⁶ is independently a direct bond or a straight or branched alkylene chain;
each R⁷ is hydrogen, alkyl, haloalkyl, alkoxyalkyl, cycloalkyl, cycloalkylalkyl, aryl, aralkyl, heterocyclyl, heterocyclylalkyl, heteroaryl or heteroarylalkyl;
each R⁸ is independently hydrogen or alkyl; and
each R⁹ is alkyl.

60. The compound of claim 59 which is (4-{[2-({[amino(imino)methyl]thio}methyl)-4-fluorophenyl]thio}-3-thienyl)methyl imidothiocarbamate.

61. A pharmaceutical composition comprising a pharmaceutically acceptable excipient and a compound of formula (I): 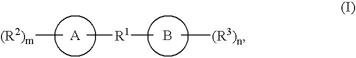
wherein:
n and m are each independently 1, 2, 3, 4, 5, 6 or 7; 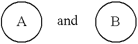
are each independently aryl or heteroaryl;
R¹ is a direct bond, —O—, —S(O)ₚ— (where p is 0, 1 or 2), —C(R⁴)₂—, —C(O)— or —N(R⁴)—;
at least one R² and at least one R³ is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
and the other R²'s and R³'s, if present, are each independently selected from the group consisting of alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, optionally substituted aryl, optionally substituted aralkyl, optionally substituted cycloalkyl, optionally substituted heterocyclyl, optionally substituted heteroaryl, or optionally substituted heteroaralkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl;
as a stereoisomer, enantiomer, tautomer thereof or mixtures thereof;
or a pharmaceutically acceptable salt, solvate or prodrug thereof.

62. A method of treating an iron disorder in a mammal, wherein the method comprises administering to the mammal a therapeutically effective amount of a compound of formula (I): 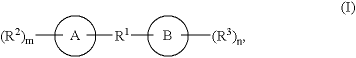
wherein:
n and m are each independently 1, 2, 3, 4, 5, 6 or 7; 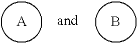
are each independently aryl or heteroaryl;
R¹ is a direct bond, —O—, —S(O)ₚ— (where p is 0, 1 or 2), —C(R⁴)₂—, —C(O)— or —N(R⁴)—;
at least one R² and at least one R³ is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
and the other R²'s and R³'s, if present, are each independently selected from the group consisting of alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)R⁹, —S(O)ₜOR⁹, —S(O)ₚR⁸, —S(O)ₜN(R⁸)₂, —R⁶—S—C(═NR⁴)N(R⁴)R⁵, —R⁶—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, optionally substituted aryl, optionally substituted aralkyl, optionally substituted cycloalkyl, optionally substituted heterocyclyl, optionally substituted heteroaryl, or optionally substituted heteroaralkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl;
as a stereoisomer, enantiomer, tautomer thereof or mixtures thereof;
or a pharmaceutically acceptable salt, solvate or prodrug thereof.

63. A method of treating a disease or condition associated with an iron disorder in a mammal, wherein the method comprises administering to the mammal in need thereof a therapeutically effective amount of a compound of formula (I): 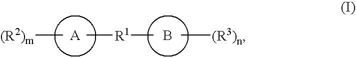
wherein:
n and m are each independently 1, 2, 3, 4, 5, 6 or 7; 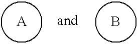
are each independently aryl or heteroaryl;
R¹ is a direct bond, —O—, —S(O)ₚ— (where p is 0, 1 or 2), —C(R⁴)₂—, —C(O)— or —N(R⁴)—;
at least one R² and at least one R³ is independently selected from the group consisting of —R⁶—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵ and —R⁶—N(R⁷)C(═NR⁴)N(R⁴)R⁵;
and the other R²'s and R³'s, if present, are each independently selected from the group consisting of alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, optionally substituted aryl, optionally substituted aralkyl, optionally substituted cycloalkyl, optionally substituted heterocyclyl, optionally substituted heteroaryl, or optionally substituted heteroaralkyl;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl;
as a stereoisomer, enantiomer, tautomer thereof or mixtures thereof;
or a pharmaceutically acceptable salt, solvate or prodrug thereof.
